SEQUENCE LISTING (7 sequences)

SEQ ID NO: 1 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - DNA, 6786 bp, Homo sapiens
atggcttgttggcctcagctgaggttgctgctgtggaagaacctcactttcagaagaagacaaacatgtcagctgctgctggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctgtatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctgagaacactaaactctacatctcccttcccgagcaaggagctggctgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatatcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccatcaaacaaatgaagaaacacctaaagctggccaaggacagttctgcagatcgatttctcaacagcttgggaagatttatgacaggactggacaccaaaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctctgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacaaaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagagtgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttctaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatga

SEQ ID NO: 3 - protein, 2261 aa, Homo sapiens
  MOD_RES (780)..(780): Any amino acid
MACWPQLRLLLWKNLTFRRRQTCQLLLEVAWPLFIFLILISVRLSYPPYEQHECHFPNKAMPSAGTLPWVQGIICNANNPCFRYPTPGEAPGVVGNFNKSIVARLFSDARRLLLYSQKDTSMKDMRKVLRTLQQIKKSSSNLKLQDFLVDNETFSGFLYHNLSLPKSTVDKMLRADVILHKVFLQGYQLHLTSLCNGSKSEEMIQLGDQEVSELCGLPREKLAAAERVLRSNMDILKPILRTLNSTSPFPSKELAEATKTLLHSLGTLAQELFSMRSWSDMRQEVMFLTNVNSSSSSTQIYQAVSRIVCGHPEGGGLKIKSLNWYEDNNYKALFGGNGTEEDAETFYDNSTTPYCNDLMKNLESSPLSRIIWKALKPLLVGKILYTPDTPATRQVMAEVNKTFQELAVFHDLEGMWEELSPKIWTFMENSQEMDLVRMLLDSRDNDHFWEQQLDGLDWTAQDIVAFLAKHPEDVQSSNGSVYTWREAFNETNQAIRTISRFMECVNLNKLEPIATEVWLINKSMELLDERKFWAGIVFTGITPGSIELPHHVKYKIRMDIDNVERTNKIKDGYWDPGPRADPFEDMRYVWGGFAYLQDVVEQAIIRVLTGTEKKTGVYMQQMPYPCYVDDIFLRVMSRSMPLFMTLAWIYSVAVIIKGIVYEKEARLKETMRIMGLDNSILWFSWFISSLIPLLVSAGLLVVILKLGNLLPYSDPSVVFVFLSVFAVVTILQCFLISTLFSRANLAAACGGIIYFTLYLPYVLCVAWQDYVGFTLKIFAXLLSPVAFGFGCEYFALFEEQGIGVQWDNLFESPVEEDGFNLTTSVSMMLFDTFLYGVMTWYIEAVFPGQYGIPRPWYFPCTKSYWFGEESDEKSHPGSNQKRISEICMEEEPTHLKLGVSIQNLVKVYRDGMKVAVDGLALNFYEGQITSFLGHNGAGKTTTMSILTGLFPPTSGTAYILGKDIRSEMSTIRQNLGVCPQHNVLFDMLTVEEHIWFYARLKGLSEKHVKAEMEQMALDVGLPSSKLKSKTSQLSGGMQRKLSVALAFVGGSKVVILDEPTAGVDPYSRRGIWELLLKYRQGRTIILSTHHMDEADVLGDRIAIISHGKLCCVGSSLFLKNQLGTGYYLTLVKKDVESSLSSCRNSSSTVSYLKKEDSVSQSSSDAGLGSDHESDTLTIDVSAISNLIRKHVSEARLVEDIGHELTYVLPYEAAKEGAFVELFHEIDDRLSDLGISSYGISETTLEEIFLKVAEESGVDAETSDGTLPARRNRRAFGDKQSCLRPFTEDDAADPNDSDIDPESRETDLLSGMDGKGSYQVKGWKLTQQQFVALLWKRLLIARRSRKGFFAQIVLPAVFVCIALVFSLIVPPFGKYPSLELQPWMYNEQYTFVSNDAPEDTGTLELLNALTKDPGFGTRCMEGNPIPDTPCQAGEEEWTTAPVPQTIMDLFQNGNWTMQNPSPACQCSSDKIKKMLPVCPPGAGGLPPPQRKQNTADILQDLTGRNISDYLVKTYVQIIAKSLKNKIWVNEFRYGGFSLGVSNTQALPPSQEVNDAXKQMKKHLKLAKDSSADRFLNSLGRFMTGLDTRNNVKVWFNNKGWHAISSFLNVINNAILRANLQKGENPSHYGITAFNHPLNLTKQQLSEVAXMTTSVDVLVSICVIFAMSFVPASFVVFLIQERVSKAKHLQFISGVKPVIYWLSNFVWDMCNYVVPATLVIIIFICFQQKSYVSSTNLPVLALLLLLYGWSITPLMYPASFVFKIPSTAYVVLTSVNLFIGINGSVATFVLELFTDNKLNNINDILKSVFLIFPHFCLGRGLIDMVKNQAMADALERFGENRFVSPLSWDLVGRNLFAMAVEGVVFFLITVLIQYRFFIRPRPVNAKLSPLNDEDEDVRRERQRILDGGGQNDILEIKELTKIYRRKRKPAVDRICVGIPPGECFGLLGVNGAGKSSTFKMLTGDTTVTRGDAFLNXNSILSNIHEVHQNMGYCPQFDAITELLTGREHVEFFALLRGVPEKEVGKVGEWAIRKLGLVKYGEKYAGNYSGGNKRKLSTAMALIGGPPVVFLDEPTTGMDPKARRFLWNCALSVVKEGRSVVLTSHSMEECEALCTRMAIMVNGRFRCLGSVQHLKNRFGDGYTIVVRIAGSNPDLKPVQDFFGLAFPGSVXKEKHRNMLQYQLPSSLSSLARIFSILSQSKKRLHIEDYSVSQTTLDQVFVNFAKDQSDDDHLKDLSLHKNQTVVDVAVLTSFLQDEKVKESYV

SEQ ID NO: 4 - DNA, 2946 bp, Homo sapiens
gctttataaaggggagtttccctgcacaagctctctctcttgtctgccgccatgtgagacatgcctttcaccttccgccatgatcatgaggcttccccagccacatggaactaatgccagcagttactctgcagagatgacggagcccaagtcggtgtgtgtctcggtggatgaggtggtgtccagcaacatggaggccactgagacggacctgctgaatggacatctgaaaaaagtagataataacctcacggaagcccagcgcttctcctccttgcctcggagggcagctgtgaacattgaattcagggacctttcctattcggttcctgaaggaccctggtggaggaagaaaggatacaagaccctcctgaaaggaatttccgggaagttcaatagtggtgagttggtggccattatgggtccttccggggccgggaagtccacgctgatgaacatcctggctggatacagggagacgggcatgaagggggccgtcctcatcaacggcctgccccgggacctgcgctgcttccggaaggtgtcctgctacatcatgcaggatgacatgctgctgccgcatctcactgtgcaggaggccatgatggtgtcggcacatctgaagcttcaggagaaggatgaaggcagaagggaaatggtcaaggagatactgacagcgctgggcttgctgtcttgcgccaacacgcggaccgggagcctgtcaggtggtcagcgcaagcgcctggccatcgcgctggagctggtgaacaaccctccagtcatgttcttcgatgagcccaccagcggcctggacagcgcctcctgcttccaggtggtctcgctgatgaaagggctcgctcaagggggtcgctccatcatttgcaccatccaccagcccagcgccaaactcttcgagctgttcgaccagctttacgtcctgagtcaaggacaatgtgtgtaccggggaaaagtctgcaatcttgtgccatatttgagggatttgggtctgaactgcccaacctaccacaacccagcagattttgtcatggaggttgcatccggcgagtacggtgatcagaacagtcggctggtgagagcggttcgggagggcatgtgtgactcagaccacaagagagacctcgggggtgatgccgaggtgaacccttttctttggcaccggccctctgaagaggactcctcgtccatggaaggctgccacagcttctctgccagctgcctcacgcagttctgcatcctcttcaagaggaccttcctcagcatcatgagggactcggtcctgacacacctgcgcatcacctcgcacattgggatcggcctcctcattggcctgctgtacttggggatcgggaacgaagccaagaaggtcttgagcaactccggcttcctcttcttctccatgctgttcctcatgttcgcggccctcatgcctactgttctgacatttcccctggagatgggagtctttcttcgggaacacctgaactactggtacagcctgaaggcctactacctggccaagaccatggcagacgtgccctttcagatcatgttcccagtggcctactgcagcatcgtgtactggatgacgtcgcagccgtccgacgccgtgcgctttgtgctgtttgccgcgctgggcaccatgacctccctggtggcacagtccctgggcctgctgatcggagccgcctccacgtccctgcaggtggccactttcgtgggcccagtgacagccatcccggtgctcctgttctcggggttcttcgtcagcttcgacaccatccccacgtacctacagtggatgtcctacatctcctatgtcaggtatgggttcgaaggggtcatcctctccatctatggcttagaccgggaagatctgcactgtgacatcgacgagacgtgccacttccagaagtcggaggccatcctgcgggagctggacgtggaaaatgccaagctgtacctggacttcatcgtactcgggattttcttcatctccctccgcctcattgcctattttgtcctcaggtacaaaatccgggcagagaggtaaaacacctgaatgccaggaaacaggaagattagacactgtggccgagggcacgtctagaatcgaggaggcaagcctgtgcccgaccgacgacacagagactcttctgatccaacccctagaaccgcgttgggtttgtgggtgtctcgtgctcagccactctgcccagctgggttggatcttctctccattcccctttctagctttaactaggaagatgtaggcagattggtggtttttttttttttaacatacagaattttaaataccacaactggggcagaatttaaagctgcaacacagctggtgatgagaggcttcctcagtccagtcgctccttagcaccaggcaccgtgggtcctggatggggaactgcaagcagcctctcagctgatggctgcacagtcagatgtctggtggcagagagtccgagcatggagcgattccattttatgactgttgtttttcacattttcatctttctaaggtgtgtctcttttccaatgagaagtcatttttgcaagccaaaagtcgatcaatcgcattcattttaagaaattatacctttttagtacttgctgaagaatgattcagggtaaatcacatactttgtttagagaggcgaggggtttaaccgagtcacccagctggtctcatacatagacagcacttgtgaaggattgaatgcaggttccaggtggagggaagacgtggacaccatctccactgagccatgcagacatttttaaaagctatacaaaaaattgtgagaagacattggccaactctttcaaagtctttctttttccacgtgcttcttattttaagcgaaatatattgtttgtttcttcctaaaaaaaaaaaaaaaaaaaaaaaaa

SEQ ID NO: 5 - protein, 678 aa, Homo sapiens
MACLMAAFSVGTAMNASSYSAEMTEPKSVCVSVDEVVSSNMEATETDLLNGHLKKVDNNLTEAQRFSSLPRRAAVNIEFRDLSYSVPEGPWWRKKGYKTLLKGISGKFNSGELVAIMGPSGAGKSTLMNILAGYRETGMKGAVLINGLPRDLRCFRKVSCYIMQDDMLLPHLTVQEAMMVSAHLKLQEKDEGRREMVKEILTALGLLSCANTRTGSLSGGQRKRLAIALELVNNPPVMFFDEPTSGLDSASCFQVVSLMKGLAQGGRSIICTIHQPSAKLFELFDQLYVLSQGQCVYRGKVCNLVPYLRDLGLNCPTYHNPADFVMEVASGEYGDQNSRLVRAVREGMCDSDHKRDLGGDAEVNPFLWHRPSEEVKQTKRLKGLRKDSSSMEGCHSFSASCLTQFCILFKRTFLSIMRDSVLTHLRITSHIGIGLLIGLLYLGIGNEAKKVLSNSGFLFFSMLFLMFAALMPTVLTFPLEMGVFLREHLNYWYSLKAYYLAKTMADVPFQIMFPVAYCSIVYWMTSQPSDAVRFVLFAALGTMTSLVAQSLGLLIGAASTSLQVATFVGPVTAIPVLLFSGFFVSFDTIPTYLQWMSYISYVRYGFEGVILSIYGLDREDLHCDIDETCHFQKSEAILRELDVENAKLYLDFIVLGIFFISLRLIAYFVLRYKIRAER

SEQ ID NO: 6 - DNA, 4262 bp, Homo sapiens
agtttccgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaaggtgaagtcggcgcggggaggggtagggccaacggcctggacgccccaagggcgggcgcagatcgcggagccatggattgcactttcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcagccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattcttggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcaccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttataaaaaaaaaaaaaaaa

SEQ ID NO: 7 - protein, 1147 aa, Homo sapiens
MDEPPFSEAALEQALGEPCDLDAALLTDIEDMLQLINNQDSDFPGLFDPPYAGSGAGGTDPASPDTSSPGSLSPPPATLSSSLEAFLSGPQAAPSPLSPPQPAPTPLKMYPSMPAFSPGPGIKEESVPLSILQTPTPQPLPGALLPQSFPAPAPPQFSSTPVLGYPSPPGGFSTGSPPGNTQQPLPGLPLASPPGVPPVSLHTQVQSVVPQQLLTVTAAPTAAPVTTTVTSQIQQVPVLLQPHFIKADSLLLTAMKTDGATVKAAGLSPLVSGTTVQTGPLPTLVSGGTILATVPLVVDAEKLPINRLAAGSKAPASAQSRGEKRTAHNAIEKRYRSSINDKIIELKDLVVGTEAKLNKSAVLRKAIDYIRFLQHSNQKLKQENLSLRTAVHKSKSLKDLVSACGSGGNTDVLMEGVKTEVEDTLTPPPSDAGSPFQSSPLSLGSRGSGSGGSGSDSEPDSPVFEDSKAKPEQRPSLHSRGMLDRSRLALCTLVFLCLSCNPLASLLGARGLPSPSDTTSVYHSPGRNVLGTESRDGPGWAQWLLPPVVWLLNGLLVLVSLVLLFVYGEPVTRPHSGPAVYFWRHRKQADLDLARGDFAQAAQQLWLALRALGRPLPTSHLDLACSLLWNLIRHLLQRLWVGRWLAGRAGGLQQDCALRVDASASARDAALVYHKLHQLHTMGKHTGGHLTATNLALSALNLAECAGDAVSVATLAEIYVAAALRVKTSLPRALHFLTRFFLSSARQACLAQSGSVPPAMQWLCHPVGHRFFVDGDWSVLSTPWESLYSLAGNPVDPLAQVTQLFREHLLERALNCVTQPNPSPGSADGDKEFSDALGYLQLLNSCSDAAGAPAYSFSISSSMATTTGVDPVAKWWASLTAVVIHWLRRDEEAAERLCPLVEHLPRVLQESERPLPRAALHSFKAARALLGCAKAESGPASLTICEKASGYLQDSLATTPASSSIDKAVQLFLCDLLLVVRTSLWRQQQPPAPAPAAQGTSSRPQASALELRGFQRDLSSLRRLAQSFRPAMRRVFLHEATARLMAGASPTRTHQLLDRSLRRRAGPGGKGGAVAELEPRPTRREHAEALLLASCYLPPGFLSAPGQRVGMLAEAARTLEKLGDRRLLHDCQQMLMRLGGGTTVTSS

(19) United States
(12) Patent Application Publication
(10) Pub. No.: US 2015/0316566 A1
(43) Pub. Date: Nov. 5, 2015

(54) HDL THERAPY MARKERS

(71) Applicant: CERENIS THERAPEUTICS HOLDING SA, Labege (FR)

(72) Inventors: Jean-Louis DASSEUX, Toulouse (FR); Ronald BARBARAS, Seilh (FR)

(73) Assignee: CERENIS THERAPEUTICS HOLDING SA, Labege (FR)

(21) Appl. No.: 14/700,351

(22) Filed: Apr. 30, 2015

Related U.S. Application Data
(60) Provisional application No. 61/988,095, filed on May 2, 2014.

(51) Int. Cl. G01N 33/92; G01N 33/50; A61K 38/17; C12Q 1/68

(52) U.S. Cl. CPC G01N 33/92; C12Q 1/6883; G01N 33/5088; A61K 38/1709; C12Q 2600/158; C12Q 2600/106

(57) ABSTRACT

The present application relates to companion diagnostic assays for therapeutic agents that mimic HDL or elevate HDL expression levels. The present application also related to methods of treatment of familial hypoalphalipoproteinemias.

### 1. CROSS REFERENCE TO RELATED APPLICATIONS

The application claims the priority under 35 U.S.C. §119(e) to U.S. provisional application No. 61/988,095, filed May 2, 2014, the contents of which are incorporated by reference in their entireties.

### 2. SEQUENCE LISTING

The instant application contains a Sequence Listing which has been submitted electronically in ASCII format and is hereby incorporated by reference in its entirety. Said ASCII copy, created on Apr. 22, 2015, is named CRN-016US_SL.txt and is 57,076 bytes in size.

### 3. BACKGROUND

3.1. Overview

Circulating cholesterol is carried by plasma lipoproteins-complex particles of lipid and protein composition that transport lipids in the blood. Four major classes of lipoprotein particles circulate in plasma and are involved in the fat-transport system: chylomicrons, very low density lipoprotein (VLDL), low density lipoprotein (LDL) and high density lipoprotein (HDL). Chylomicrons constitute a short-lived product of intestinal fat absorption. VLDL and, particularly, LDL are responsible for the delivery of cholesterol from the liver (where it is synthesized or obtained from dietary sources) to extrahepatic tissues, including the arterial walls. HDL, by contrast, mediates reverse cholesterol transport (RCT), the removal of cholesterol lipids, in particular from extrahepatic tissues to the liver, where it is stored, catabolized, eliminated or recycled. HDL also plays a beneficial role in inflammation, transporting oxidized lipids and interleukin, which may in turn reduce inflammation in blood vessel walls.

Lipoprotein particles have a hydrophobic core comprised of cholesterol (normally in the form of a cholesteryl ester) and triglycerides. The core is surrounded by a surface coat comprising phospholipids, unesterified cholesterol and apolipoproteins. Apolipoproteins mediate lipid transport, and some may interact with enzymes involved in lipid metabolism. At least ten apolipoproteins have been identified, including: ApoA-I, ApoA-II, ApoA-IV, ApoA-V, ApoB, ApoC-I, ApoC-II, ApoC-III, ApoD, ApoE, ApoJ and ApoH. Other proteins such as LCAT (lecithin:cholesterol acyltransferase), CETP (cholesteryl ester transfer protein), PLTP (phospholipid transfer protein) and PON (paraoxonase) are also found associated with lipoproteins.

Cardiovascular diseases such as coronary heart disease, coronary artery disease and atherosclerosis are linked overwhelmingly to elevated serum cholesterol levels. For example, atherosclerosis is a slowly progressive disease characterized by the accumulation of cholesterol (and cholesterol esters) within the arterial wall. Accumulation of cholesterol and cholesterol esters in macrophages lead to the formation of foam cells, a hallmark of atherosclerotic plaques. Compelling evidence supports the theory that lipids deposited in atherosclerotic lesions are derived primarily from plasma LDLs; thus, LDLs have popularly become known as “bad” cholesterol. In contrast, HDL serum levels correlate inversely with coronary heart disease. Indeed, high serum levels of HDLs are regarded as a negative risk factor. It is hypothesized that high levels of plasma HDLs are not only protective against coronary artery disease, but may actually induce regression of atherosclerotic plaque (see, e.g., Badimon et al., 1992, Circulation 86 (Suppl. III):86-94; Dansky and Fisher, 1999, Circulation 100:1762-63; Tangirala et al., 1999, Circulation 100(17):1816-22; Fan et al., 1999, Atherosclerosis 147(1):139-45; Deckert et al., 1999, Circulation 100(11):1230-35; Boisvert et al., 1999, Arterioscler. Thromb. Vasc. Biol. 19(3):525-30; Benoit et al., 1999, Circulation 99(1):105-10; Holvoet et al., 1998, J. Clin. Invest. 102(2):379-85; Duverger et al., 1996, Circulation 94(4):713-17; Miyazaki et al., 1995, Arterioscler. Thromb. Vasc. Biol. 15(11):1882-88; Mezdour et al., 1995, Atherosclerosis 113(2):237-46; Liu et al., 1994, J. Lipid Res. 35(12):2263-67; Plump et al., 1994, Proc. Nat. Acad. Sci. USA 91(20):9607-11; Paszty et al., 1994, J. Clin. Invest. 94(2):899-903; She et al, 1992, Chin. Med. J. (Engl). 105(5):369-73; Rubin et al., 1991, Nature 353(6341):265-67; She et al., 1990, Ann. NY Acad. Sci. 598:339-51; Ran, 1989, Chung Hua Ping Li Hsueh Tsa Chih (also translated as: Zhonghua Bing Li Xue Za Zhi) 18(4):257-61; Quezado et al., 1995, J. Pharmacol. Exp. Ther. 272(2):604-11; Duverger et al., 1996, Arterioscler. Thromb. Vasc. Biol. 16(12):1424-29; Kopfler et al., 1994, Circulation; 90(3):1319-27; Miller et al., 1985, Nature 314(6006):109-11; Ha et al., 1992, Biochim. Biophys. Acta 1125(2):223-29; Beitz et al., 1992, Prostaglandins Leukot. Essent. Fatty Acids 47(2):149-52). As a consequence, HDLs have popularly become known as “good” cholesterol, (see, e.g., Zhang, et al., 2003 Circulation 108:661-663).

The “protective” role of HDL has been confirmed in a number of studies (e.g., Miller et al., 1977, Lancet 1(8019):965-68; Whayne et al., 1981, Atherosclerosis 39:411-19). In these studies, the elevated levels of LDL appear to be associated with increased cardiovascular risk, whereas high HDL levels seem to confer cardiovascular protection. In vivo studies have further demonstrated the protective role of HDL, showing that HDL infusions into rabbits may hinder the development of cholesterol induced arterial lesions (Badimon et al., 1989, Lab. Invest. 60:455-61) and/or induce their regression (Badimon et al., 1990, J. Clin. Invest. 85:1234-41). In a post hoc analysis of the Treating to New Target (TNT) study, HDL-chol was predictive of major cardiovascular event in patients treated with statins, even in patients whose LDL-chol was less than 70 mg/dl.

In recent clinical trials, niacin and two CETP-inhibitors (Torcetrapib (Pfizer) and Dalcetrapib (Roche)) failed to reduce the incidence of coronary events over a long term treatment although some of these studies may suffer from some confounding factors (Boden et al., 2011, N Engl J Med 365:2255-2267; HPS2-THRIVE Collaborative Group, 2013, Eur. Heart J. 34:1279-1291; Barter et al., 2007, N Engl J Med 357:2109-2122; Schwartz et al., 2012, N. Engl. J. Med. 367:2089-2099). Two Mendelian genetic studies questioned the link between HDL-cholesterol and risk of cardiovascular disease (Voight et al., Lancet DOI:10.1016/S0140-6736(12)60312-2, published online May 17, 2012; Holmes et al., Eur Heart J doi:10.1093/eurheartj/eht571, published online Jan. 27, 2014). These studies further emphasize the idea that the number of functional HDL particles and enhancement of reverse lipid transport are the important factors for the prevention of cardiovascular events rather than an elevation of HDL cholesterol (HDL-c) (Barter et al., 2007, N Engl J Med 357:2109-22; Group et al., 2010, N Engl J Med 362:1563-74; Nissen et al., 2007, The New England journal of medicine 356:1304-16). Indeed, in the MESA clinical trial with more than 5,000 patients, the best factor that correlated with the incidence of CHD and cardiovascular events was HDL particle number rather than the cholesterol content of the HDL fraction (i.e. HDL-c) (Mackey et al., 2012, Journal of the American College of Cardiology 60:508-16; van der Steeg et al., 2008, Journal of the American College of Cardiology 51:634-42). In the setting of potent statin therapy, HDL particle number may be a better marker of residual risk than chemically-measured HDL-chol or ApoA-I (Mora et al. 2013, Circulation DOI: 10.1161/CIRCULATIONAHA.113.002671).

3.2. Reverse Lipid Transport, HDL and Apolipoprotein A-I

The protective function of HDL particles can be explained by their role in the reverse lipid transport (RLT) pathway, also known as the reverse cholesterol transport (RCT) pathway. The RLT (Tall, 1998, Eur Heart J 19:A31-5) pathway is responsible for removal of cholesterol from arteries and its transport to the liver for elimination from the body in mainly four basic steps.

The first step is the removal of cholesterol from arteries by the nascent HDL particle in a process termed “cholesterol removal.” Cholesterol is a membrane constituent that maintains structural domains that are important in the regulation of vesicular trafficking and signal transduction. In most cells, cholesterol is not catabolized. Thus, the regulation of cellular sterol efflux plays a crucial role in cellular sterol homeostasis. Cellular sterol can efflux to extracellular sterol acceptors by both non-regulated, passive diffusion mechanisms as well as by an active, regulated, energy-dependent process mediated by receptors, such as the ABCA1 and ABCG1 transporters.

LCAT, the key enzyme in RCT, is produced by the intestine and the liver and circulates in plasma mainly associated with the HDL fraction. LCAT converts cell-derived cholesterol to cholesteryl esters, which are sequestered in HDL destined for removal (see Jonas 2000, Biochim. Biophys. Acta 1529(1-3):245-56). Cholesteryl ester transfer protein (CETP) and phospholipid transfer protein (PLTP) contribute to further remodeling of the circulating HDL population. CETP moves cholesteryl esters made by LCAT to other lipoproteins, particularly ApoB-comprising lipoproteins, such as VLDL and LDL. PLTP supplies lecithin to HDL. HDL triglycerides are catabolized by the extracellular hepatic triglyceride lipase, and lipoprotein cholesterol is removed by the liver via several mechanisms.

The functional characteristics of HDL particles are mainly determined by their major apolipoprotein components such as ApoA-I and ApoA-II. Minor amounts of ApoC-I, ApoC-II, ApoC-III, ApoD, ApoA-IV, ApoE, and ApoJ have also been observed associated with HDL. HDL exists in a wide variety of different sizes and different mixtures of the above-mentioned constituents, depending on the status of remodeling during the metabolic RCT cascade or pathway.

Each HDL particle usually comprises at least 1 molecule, and usually two to 4 molecules, of ApoA-I. HDL particles may also comprise only ApoE (gamma-LpE particles), which are known to also be responsible for cholesterol efflux, as described by Prof. Gerd Assmann (see, e.g., von Eckardstein et al., 1994, Curr Opin Lipidol. 5(6):404-16). ApoA-I is synthesized by the liver and small intestine as preproApolipoprotein A-I, which is secreted as proApolipoprotein A-I (proApoA-I) and rapidly cleaved to generate the plasma form of ApoA-I, a single polypeptide chain of 243 amino acids (Brewer et al., 1978, Biochem. Biophys. Res. Commun. 80:623-30). PreproApoA-I that is injected experimentally directly into the bloodstream is also cleaved into the plasma form of ApoA-I (Klon et al., 2000, Biophys. J. 79(3):1679-85; Segrest et al., 2000, Curr. Opin. Lipidol. 11(2):105-15; Segrest et al., 1999, J. Biol. Chem. 274 (45):31755-58).

ApoA-I comprises 6 to 8 different 22-amino acid alpha-helices or functional repeats spaced by a linker moiety that is frequently proline. The repeat units exist in amphipathic helical conformation (Segrest et al., 1974, FEBS Lett. 38: 247-53) and confer the main biological activities of ApoA-I, i.e., lipid binding and lecithin cholesterol acyl transferase (LCAT) activation.

ApoA-I forms three types of stable complexes with lipids: small, lipid-poor complexes referred to as pre-beta-1 HDL; flattened discoidal particles comprising polar lipids (phospholipid and cholesterol) referred to as pre-beta-2 HDL; and spherical particles, comprising both polar and nonpolar lipids, referred to as spherical or mature HDL (HDL₃ and HDL₂). Most HDL in the circulating population comprises both ApoA-I and ApoA-II (the “AI/AII-HDL fraction”). However, the fraction of HDL comprising only ApoA-I (the “AI-HDL fraction”) appears to be more effective in RCT. Certain epidemiologic studies support the hypothesis that the ApoA-I-HDL fraction is anti-atherogenic (Parra et al., 1992, Arterioscler. Thromb. 12:701-07; Decossin et al., 1997, Eur. J. Clin. Invest. 27:299-307).

HDL particles are made of several populations of particles that have different sizes, lipid composition and apolipoprotein composition. They can be separated according to their properties, including their hydrated density, apolipoprotein composition and charge characteristics. For example, the pre-beta-HDL fraction is characterized by a lower surface charge than mature alpha-HDL. Because of this charge difference, pre-beta-HDL and mature alpha-HDL have different electrophoretic mobilities in agarose gel (David et al., 1994, J. Biol. Chem. 269(12):8959-8965).

The metabolism of pre-beta-HDL and mature alpha-HDL also differs. Pre-beta-HDL has two metabolic fates: either removal from plasma and catabolism by the kidney or remodeling to medium-sized HDL that are preferentially degraded by the liver (Lee et al., 2004, J. Lipid Res. 45(4):716-728).

Although the mechanism for cholesterol transfer from the cell surface (i.e., cholesterol efflux) is unknown, it is believed that the lipid-poor complex, pre-beta-1 HDL, is the preferred acceptor for cholesterol transferred from peripheral tissue involved in RCT (see Davidson et al., 1994, J. Biol. Chem. 269:22975-82; Bielicki et al., 1992, J. Lipid Res. 33:1699-1709; Rothblat et al., 1992, J. Lipid Res. 33:1091-97; and Kawano et al., 1993, Biochemistry 32:5025-28; Kawano et al., 1997, Biochemistry 36:9816-25). During this process of cholesterol recruitment from the cell surface, pre-beta-1 HDL is rapidly converted to pre-beta-2 HDL. PLTP may increase the rate of pre-beta-2 HDL disc formation, but data indicating a role for PLTP in RCT are lacking. LCAT reacts preferentially with discoidal, small (pre-beta) and spherical (i.e., mature) HDL, transferring the 2-acyl group of lecithin or other phospholipids to the free hydroxyl residue of cholesterol to generate cholesteryl esters (retained in the HDL) and lysolecithin. The LCAT reaction requires ApoA-I as an activator; i.e., ApoA-I is the natural cofactor for LCAT. The conversion of cholesterol sequestered in the HDL to its ester prevents re-entry of cholesterol into the cells, the net result being that cholesterol is removed from the cell as the gradient of the cell and the HDL is maintained.

Cholesteryl esters in the mature HDL particles in the ApoA-I-HDL fraction (i.e., comprising ApoA-I and no ApoA-II) are removed by the liver and processed into bile more effectively than those derived from HDL comprising both ApoA-I and ApoA-II (the AI/AII-HDL fraction). This may be owed, in part, to the more effective binding of ApoA-I-HDL to the hepatocyte membrane. The existence of an HDL receptor has been hypothesized, and a scavenger receptor, class B, type I (SR-BI) has been identified as an HDL receptor (Acton et al., 1996, Science 271:518-20; Xu et al., 1997, Lipid Res. 38:1289-98). SR-BI is expressed most abundantly in steroidogenic tissues (e.g., the adrenals), and in the liver (Landschulz et al., 1996, J. Clin. Invest. 98:984-95; Rigotti et al., 1996, J. Biol. Chem. 271:33545-49). For a review of HDL receptors, see Broutin et al., 1988, Anal. Biol. Chem. 46:16-23.

Initial lipidation by ATP-binding cassette transporter AI (ABCA1) appears to be critical for plasman HDL formation and for the ability of pre-beta-HDL particles to effect cholesterol efflux (Lee and Parks, 2005, Curr. Opin. Lipidol. 16(1):19-25). According to these authors, this initial lipidation enables pre-beta-HDL to function more efficiently as a cholesterol acceptor and prevents ApoA-I from rapidly associating with pre-existing plasman HDL particles, resulting in greater availability of pre-beta-HDL particles for cholesterol efflux.

ABCA1 deficiency is one of the underlying causes of familial primary hypoalphalipoproteinemia. Familial primary hypoalphalipoproteinemia is caused by genetic defect in one of the genes responsible for HDL synthesis/maturation, such as ABCA1, and is associated with a very low number of high-density lipoprotein (HDL)-particles, also reflected in a very low plasma concentration of apolipoprotein A-I (ApoA-I). The disease is also generally associated with a positive family history of low HDL-cholesterol (HDL-C) or premature cardiovascular disease.

Homozygous ABCA1 deficiency, also called Tangier disease, is characterized by severe plasma deficiency or absence of HDL, apolipoprotein A-I (ApoA-I) and by accumulation of cholesteryl esters in tissues throughout the body (Puntoni et al, 2012). Subjects with Tangier disease present with large, yellow-orange tonsils and/or neuropathy. Other clinical features include hepatomegaly, splenomegaly, premature myocardial infarction or stroke, thrombocytopenia, anemia, and corneal opacities.

Recently, a second ATP-binding cassette transporter G1 (ABCG1) was described as mediating intracellular cholesterol homeostasis. The expression of ABCG1 enhances cholesterol efflux through interactions with predominantly spherical, cholesterol-containing medium- to very large-HDL particles, as well as large discoidal HDL particles. Larger particles are similarly effective as smaller HDL particles as acceptors from ABCG1.

The ATP-binding cassette transporters ABCA1 and ABCG1 are increased by liver X receptor transcription factors, (Costet et al., 2000, J Biol Chem 275:28240-5; Kennedy et al., 2001, J Biol Chem 276:39438-47) which play a pivotal role in modulating cholesterol efflux by both the ABCA1 and ABCG1 transporters. In vivo, liver X receptors are activated by specific oxysterols in cholesterol-loaded cells ABCA1 and ABCG1 are key target genes of liver X receptors in macrophages (Janowski et al., 1996, Nature 383:728-31). Although ABCA1 promotes cholesterol efflux to cholesterol-deficient and phospholipid-depleted ApoA-I and apoE complexes, ABCG1 promotes efflux to HDL particles (Duong et al., 2006, Journal of lipid research 47:832-43; Mulya et al., 2007, Arteriosclerosis, thrombosis, and vascular biology 27:1828-36; Wang et al., 2004, Proceedings of the National Academy of Sciences of the United States of America 101:9774-9). Increased expression of the ABCA1 and ABCG1 transporters is associated with redistribution of cholesterol from the inner to the outer leaflet of the plasma membrane, facilitating cholesterol efflux from cholesterol-loaded foam cells to HDL particles (Pagler et al., 2011, Circulation research 108:194-200). The coordinated participation of ABCA1 and ABCG1 in mediating macrophage cholesterol efflux has been demonstrated from animal studies. A single deficiency of ABCA1 in mice results in a moderate increase in atherosclerosis, and deficiency of ABCG1 has no effect; however, combined deficiency resulted in markedly accelerated lesion development (Yvan-Charvet et al., 2007, The Journal of clinical investigation 117:3900-8). Double-knockout macrophages showed markedly defective cholesterol efflux to HDL and ApoA-I and increased inflammatory responses when treated with lipopolysaccharide (Yvan-Charvet et al., 2008, Circulation 118:1837-47).

Cholesterol homeostasis has also recently been investigated with microRNAs (miRNA), which are small endogenous non-protein-coding RNAs that are posttranscriptional regulators of genes involved in physiological processes (Rayner et al., 2010, Science (New York, N.Y.) 328:1570-3; Najafi-Shoushtari et al., 2010, Science (New York, N.Y.) 328:1566-9; Marquart et al., 2010, PNAS). MiR-33, an intronic miRNA located within the gene encoding sterol-regulatory element binding factor-2, inhibits hepatic expression of both ABCA1 and ABCG1, reducing HDL-C concentrations (Yvan-Charvet et al., 2008, Circulation 118:1837-47; Marquart et al., 2010, PNAS), as well as ABCA1 expression in macrophages, thus resulting in decreased cholesterol efflux (Yvan-Charvet et al., 2008, Circulation 118:1837-47). Antagonism of MiR-33 by oligonucleotides raised HDL-C and reduced atherosclerosis in a mouse model (Rayner et al., 2011, The Journal of Clinical Investigation 121:2921-31).

ABCA1 as well as ABCG1 are highly regulated by cellular cholesterol content. Cellular lipid over-load leads to the formation of oxysterols, which activate nuclear liver X receptors (LXR) to induce the transcription of ABCA1 and ABCG1 and hence cholesterol efflux (Jakobsson et al., 2012, Trends in pharmacological sciences 33:394-404). Thus, the cholesterol efflux is determined both by the extra-cellular concentration and composition of HDL particles and by the activity of the ABC transporters.

Interestingly, it seems that the ABCA1 expression was down-regulated by the presence in the cell medium of already loaded HDL particles (Langmann et al., 1999, Biochemical and biophysical research communications 257:29-33).

The cholesterol efflux as a key regulator of cellular cholesterol homeostasis exerts important regulatory steps on many cellular functions such as proliferation and mobilization of hematopoietic stem cells (Tall et al., 2012, Arterioscler Thromb Vasc Biol 32:2547-52)

The ATP-binding cassette transporter G4 (ABCG4) mediates cholesterol efflux to HDL which lead to megakaryocyte proliferation (Murphy et al., 2013, Nature medicine 19:586-94).

Cholesterol efflux regulates the inflammatory responses to monocytes and macrophages (Westerterp et al., 2013, Circulation research 112:1456-65), the expansion of lymphocytes (Sorci-Thomas et al., 2012, Arterioscler Thromb Vasc Biol 32:2561-5), the nitric oxide (NO) production by endothelial-nitric oxide synthase (eNOS) (Terasaka et al., 2010, Arterioscler Thromb Vasc Biol 30:2219-25) and insulin production from pancreatic R-cells (Kruit et al., 2012, Diabetes 61:659-64).

CETP may also play a role in RCT. Changes in CETP activity or its acceptors, VLDL and LDL, play a role in “remodeling” the HDL population. For example, in the absence of CETP, the HDLs become enlarged particles that are not cleared. (For reviews of RCT and HDLs, see Fielding and Fielding, 1995, J. Lipid Res. 36:211-28; Barrans et al., 1996, Biochem. Biophys. Acta 1300:73-85; Hirano et al., 1997, Arterioscler. Thromb. Vasc. Biol. 17(6):1053-59).

HDL also plays a role in the reverse transport of other lipids and apolar molecules, and in detoxification, i.e., the transport of lipids from cells, organs, and tissues to the liver for catabolism and excretion. Such lipids include sphingomyelin (SM), oxidized lipids, and lysophophatidylcholine. For example, Robins and Fasulo (1997, J. Clin. Invest. 99:380-84) have shown that HDLs stimulate the transport of plant sterol by the liver into bile secretions.

The major component of HDL, ApoA-I, can associate with SM in vitro. When ApoA-I is reconstituted in vitro with bovine brain SM (BBSM), a maximum rate of reconstitution occurs at 28° C., the temperature approximating the phase transition temperature for BBSM (Swaney, 1983, J. Biol. Chem. 258(2), 1254-59). At BBSM:ApoA-I ratios of 7.5:1 or less (wt/wt), a single reconstituted homogeneous HDL particle is formed that comprises three ApoA-I molecules per particle and that has a BBSM:ApoA-I molar ratio of 360:1. It appears in the electron microscope as a discoidal complex similar to that obtained by recombination of ApoA-I with phosphatidylcholine at elevated ratios of phospholipid/protein. At BBSM:ApoA-I ratios of 15:1 (wt/wt), however, larger-diameter discoidal complexes form that have a higher phospholipid:protein molar ratio (535:1). These complexes are significantly larger, more stable, and more resistant to denaturation than ApoA-I complexes formed with phosphatidylcholine.

Sphingomyelin (SM) is elevated in early cholesterol acceptors (pre-beta-HDL and gamma-migrating ApoE-comprising lipoprotein), suggesting that SM might enhance the ability of these particles to promote cholesterol efflux (Dass and Jessup, 2000, J. Pharm. Pharmacol. 52:731-61; Huang et al., 1994, Proc. Natl. Acad. Sci. USA 91:1834-38; Fielding and Fielding 1995, J. Lipid Res. 36:211-28).

3.3. Protective Mechanism of HDL and ApoA-I

Studies of the protective mechanism(s) of HDL have focused on Apolipoprotein A-I (ApoA-I), the major component of HDL. High plasma levels of ApoA-I are associated with absence or reduction of coronary lesions (Maciejko et al., 1983, N. Engl. J. Med. 309:385-89; Sedlis et al., 1986, Circulation 73:978-84).

The infusion of ApoA-I or of HDL in experimental animals exerts significant biochemical changes, as well as reduces the extent and severity of atherosclerotic lesions. After an initial report by Maciejko and Mao (1982, Arteriosclerosis 2:407a), Badimon et al., (1989, Lab. Invest. 60:455-61; 1989, J. Clin. Invest. 85:1234-41) found that they could significantly reduce the extent of atherosclerotic lesions (reduction of 45%) and their cholesterol ester content (reduction of 58.5%) in cholesterol-fed rabbits, by infusing HDL (d=1.063-1.325 g/ml). They also found that the infusions of HDL led to a close to a 50% regression of established lesions. (Esper et al. 1987, Arteriosclerosis 7:523a) have shown that infusions of HDL can markedly change the plasma lipoprotein composition of Watanabe rabbits with inherited hypercholesterolemia, which develop early arterial lesions. In these rabbits, HDL infusions can more than double the ratio between the protective HDL and the atherogenic LDL. Recently, several infusions of CER-001, a recombinant human apolipoprotein A-I engineered pre-β HDL was able to reduce vascular inflammation and promote regression of diet-induced atherosclerosis in LDL receptor knock-out mice, a preclinical model for familial Hypercholesterolemia (HDLTardy et al., Atherosclerosis 232 (2014) 110-118).

The potential of HDL to prevent arterial disease in animal models has been further underscored by the observation that ApoA-I can exert a fibrinolytic activity in vitro (Saku et al., 1985, Thromb. Res. 39:1-8). Ronneberger (1987, Xth Int. Congr. Pharmacol., Sydney, 990) demonstrated that ApoA-I can increase fibrinolysis in beagle dogs and in Cynomologous monkeys. A similar activity can be noted in vitro on human plasma. Ronneberger was able to confirm a reduction of lipid deposition and arterial plaque formation in ApoA-I treated animals.

In vitro studies indicate that complexes of ApoA-I and lecithin can promote the efflux of free cholesterol from cultured arterial smooth muscle cells (Stein et al., 1975, Biochem. Biophys. Acta, 380:106-18). By this mechanism, HDL can also reduce the proliferation of these cells (Yoshida et al., 1984, Exp. Mol Pathol. 41:258-66).

Infusion therapy with HDL comprising ApoA-I or ApoA-I mimetic peptides has also been shown to regulate plasman HDL levels by the ABCA1 transporter, leading to efficacy in the treatment of cardiovascular disease (see, e.g., Brewer et al., 2004, Arterioscler. Thromb. Vasc. Biol. 24:1755-1760).

Two naturally occurring human polymorphism of ApoA-I have been isolated in which an arginine residue is substituted with cysteine. In Apolipoprotein A-I_{Milano} (ApoA-I_{M}), this substitution occurs at residue 173, whereas in Apolipoprotein A-I_{Paris} (ApoA-I_{P}), this substitution occurs at residue 151 (Franceschini et al., 1980, J. Clin. Invest. 66:892-900; Weisgraber et al., 1983, J. Biol. Chem. 258:2508-13; Bruckert et al., 1997, Atherosclerosis 128:121-28; Daum et al., 1999, J. Mol. Med. 77:614-22; Klon et al., 2000, Biophys. J. 79(3):1679-85). Yet a further naturally occurring human polymorphism of ApoA-I has been isolated, in which a leucine is substituted with an arginine at position 144. This polymorphism has been termed Apolipoprotein A-I Zaragoza (ApoA-I_{Z}) and is associated with severe hypoalphalipoproteinemia and an enhanced effect of high density lipoprotein (HDL) reverse cholesterol transport (Recalde et al., 2001, Atherosclerosis 154(3):613-623; Fiddyment et al., 2011, Protein Expr. Purif. 80(1):110-116).

Reconstituted HDL particles comprising disulfide-linked homodimers of either ApoA-I_{M} or ApoA-I_{P} are similar to reconstituted HDL particles comprising wild-type ApoA-I in their ability to clear dimyristoylphosphatidylcholine (DMPC) emulsions and their ability to promote cholesterol efflux (Calabresi et al., 1997b, Biochemistry 36:12428-33; Franceschini et al., 1999, Arterioscler. Thromb. Vasc. Biol. 19:1257-62; Daum et al., 1999, J. Mol. Med. 77:614-22). In both mutations, heterozygous individuals have decreased levels of HDL but paradoxically, are at a reduced risk for atherosclerosis (Franceschini et al., 1980, J. Clin. Invest. 66:892-900; Weisgraber et al., 1983, J. Biol. Chem. 258:2508-13; Bruckert et al., 1997, Atherosclerosis 128:121-28). Reconstituted HDL particles comprising either variant are capable of LCAT activation, although with decreased efficiency when compared with reconstituted HDL particles comprising wild-type ApoA-I (Calabresi et al., 1997, Biochem. Biophys. Res. Commun. 232:345-49; Daum et al., 1999, J. Mol. Med. 77:614-22).

The ApoA-I_{M} mutation is transmitted as an autosomal dominant trait; eight generations of carriers within a family have been identified (Gualandri et al., 1984, Am. J. Hum. Genet. 37:1083-97). The status of an ApoA-I_{M} carrier individual is characterized by a remarkable reduction in HDL-cholesterol level. In spite of this, carrier individuals do not apparently show any increased risk of arterial disease. Indeed, by examination of genealogical records, it appears that these subjects may be “protected” from atherosclerosis (Sirtori et al., 2001, Circulation, 103: 1949-1954; Roma et al., 1993, J. Clin. Invest. 91(4):1445-520).

The mechanism of the possible protective effect of ApoA-I_{M} in carriers of the mutation seems to be linked to a modification in the structure of the mutant ApoA-I_{M}, with loss of one alpha-helix and an increased exposure of hydrophobic residues (Franceschini et al., 1985, J. Biol. Chem. 260:1632-35). The loss of the tight structure of the multiple alpha-helices leads to an increased flexibility of the molecule, which associates more readily with lipids, compared to normal ApoA-I. Moreover, lipoprotein complexes are more susceptible to denaturation, thus suggesting that lipid delivery is also improved in the case of the mutant.

Bielicki, et al. (1997, Arterioscler. Thromb. Vasc. Biol. 17 (9):1637-43) has demonstrated that ApoA-I_{M} has a limited capacity to recruit membrane cholesterol compared with wild-type ApoA-I. In addition, nascent HDL formed by the association of ApoA-I_{M} with membrane lipids was predominantly 7.4-nm particles rather than larger 9- and 11-nm complexes formed by wild-type ApoA-I. These observations indicate that the Arg₁₇₃→Cys₁₇₃ substitution in the ApoA-I primary sequence interfered with the normal process of cellular cholesterol recruitment and nascent HDL assembly. The mutation is apparently associated with a decreased efficiency for cholesterol removal from cells. Its antiatherogenic properties may therefore be unrelated to RCT. It could also be due to its ability to limit the maturation of HDL to small particles.

The most striking structural change attributed to the Arg₁₇₃→Cys₁₇₃ substitution is the dimerization of ApoA-I_{M} (Bielicki et al., 1997, Arterioscler. Thromb. Vasc. Biol. 17 (9):1637-43). ApoA-I_{M} can form homodimers with itself and heterodimers with ApoA-II. Studies of blood fractions comprising a mixture of apolipoproteins indicate that the presence of dimers and complexes in the circulation may be responsible for an increased elimination half-life of apolipoproteins. Such an increased elimination half-life has been observed in clinical studies of carriers of the mutation (Gregg et al., 1988, NATO ARW on Human Apolipoprotein Mutants: From Gene Structure to Phenotypic Expression, Limone S G). Other studies indicate that ApoA-I_{M} dimers (ApoA-I_{M}/ApoA-I_{M}) act as an inhibiting factor in the interconversion of HDL particles in vitro (Franceschini et al., 1990, J. Biol. Chem. 265:12224-31).

3.4. Current Treatments for Dyslipidemia and Related Disorders

Dyslipidemic disorders are diseases associated with elevated serum cholesterol and triglyceride levels and lowered serum HDL:LDL ratios, and include hyperlipidemia, especially hypercholesterolemia, coronary heart disease, coronary artery disease, vascular and perivascular diseases, and cardiovascular diseases such as atherosclerosis. Syndromes associated with atherosclerosis such as transient ischemic attack or intermittent claudication, caused by arterial insufficiency, are also included. A number of treatments are currently available for lowering the elevated serum cholesterol and triglycerides associated with dyslipidemic disorders. However, each has its own drawbacks and limitations in terms of efficacy, side-effects and qualifying patient population. Some dyslipidemic disorders are associated with HDL deficiency due to mutations in the genes responsible for HDL synthesis, maturation or elimination, such as but not limited to Tangier's disease, ABCA1 deficiency, ApoA-I deficiency, LCAT deficiency or Fish-eye disease. These disorders can be regrouped under the term of Familial Primary Hypoalphalipoproteinemia (FPHA).

Bile-acid-binding resins are a class of drugs that interrupt the recycling of bile acids from the intestine to the liver; e.g., cholestyramine (Questran Light®, Bristol-Myers Squibb), colestipol hydrochloride (Colestid®, The Upjohn Company), and colesevelam hydrochloride (Welchol®, Daiichi-Sankyo Company). When taken orally, these positively-charged resins bind to the negatively charged bile acids in the intestine. Because the resins cannot be absorbed from the intestine, they are excreted carrying the bile acids with them. The use of such resins at best, however, only lowers serum cholesterol levels by about 20%, and is associated with gastrointestinal side-effects, including constipation and certain vitamin deficiencies. Moreover, since the resins bind other drugs, other oral medications must be taken at least one hour before or four to six hours subsequent to ingestion of the resin; thus, complicating heart patient's drug regimens.

Statins are cholesterol lowering agents that block cholesterol synthesis by inhibiting HMGCoA reductase, the key enzyme involved in the cholesterol biosynthetic pathway. Statins, e.g., lovastatin (Mevacor®), simvastatin (Zocor®), pravastatin (Pravachol®), fluvastatin (Lescol®), pitavastatin (Livalo®) and atorvastatin (Lipitor®), are sometimes used in combination with bile-acid-binding resins. Statins significantly reduce serum cholesterol and LDL-serum levels, and slow progression of coronary atherosclerosis. However, serum HDL cholesterol levels are only moderately increased. The mechanism of the LDL lowering effect may involve both reduction of VLDL concentration and induction of cellular expression of LDL-receptor, leading to reduced production and/or increased catabolism of LDLs. Side effects, including liver and kidney dysfunction are associated with the use of these drugs (The Physicians Desk Reference, 56^{th} Ed., 2002, Medical Economics).

Niacin (nicotinic acid) is a water soluble vitamin B-complex used as a dietary supplement and antihyperlipidemic agent. Niacin diminishes production of VLDL and is effective at lowering LDL. In some cases, it is used in combination with bile-acid binding resins. Niacin can increase HDL when used at adequate doses, however, its usefulness is limited by serious side effects when used at such high doses. Niaspan® is a form of extended-release niacin that produces fewer side effects than pure niacin. Niacin/Lovastatin (Nicostatin®) is a formulation containing both niacin and lovastatin and combines the benefits of each drug. The ARBITER 6-HALTS (Arterial Biology for the Investigation of the Treatment Effects of Reducing Cholesterol 6-HDL and LDL Treatment Strategies in Atherosclerosis) trial showed that niacin not only favorably modified lipid profiles, but also reduced plaque formation in carotid and coronary arteries (Villines et al., 2010, J Am Coll Cardiol 55:2721-6). Unfortunately, the large outcome trial AIM-HIGH (Atherothrombosis Intervention in Metabolic Syndrome with Low HDL/High Triglycerides), supported by the National Institutes of Health, was stopped after a little more than 3000 patients had been recruited, because of futility (Investigators et al., 2011, N Engl J Med 365:2255-67). The HPS-THRIVE (Heart Protection Study 2—Treatment of HDL to Reduce the Incidence of Vascular Events) trial, which investigated the effect of extended-release niacin in combination with laropiprant (a prostaglandin D2 receptor antagonist to reduce the incidence of flushing) in addition to simvastatin in 25 673 patients at high cardiovascular risk, have shown no significant benefit of the niacin-laropiprant combination on major vascular events (Group, 2013, Eur Heart J 34:1279-91).

A novel class of HDL-cholesterol increasing drugs is the CETP inhibitors. By reducing the transfert of cholesterol ester from the HDL to VLDL or LDL, CETP inhibitors produce marked and consistent increase of plasman HDL-cholesterol levels between 30 to 140% (ref). Associated to statin the LDL-cholesterol remains unchanged (Dalcetrapib) or decrease further by about 40% (torcetrapib, anacetrapib, or evacetrapib). In the ILLUMINATE (Investigation of Lipid Level Management to Understand its Impact in Atherosclerotic Events) trial the addition of torcetrapib to 80 mg of atorvastatin to 15 067 patients was associated to an increase of the mortality and morbidity (Barter et al., 2007, N Engl J Med 357:2109-22) despite an HDL-cholesterol increase of 80% and a LDL-cholesterol decrease of 25% as compared to Atorvastatin alone (Barter et al., 2007, N Engl J Med 357:2109-22). Two other trials, the RADIANCE 2 (Rating Atherosclerotic Disease Change by Imaging with a New Cholesteryl-Ester-Transfer Protein Inhibitor) trial (Bots et al., 2007, Lancet 370:153-60), which used B-mode carotid ultrasound, as well as in the ILLUSTRATE (Investigation of Lipid Level Management Using Coronary Ultrasound to Assess Reduction of Atherosclerosis by CETP Inhibition and HDL Elevation) trial (Nissen et al., 2007, N Engl J Med 356:1304-16) which used coronary intravascular ultrasound, torcetrapib did not reduce carotid intima-media thickness, nor did it decrease coronary plaque volume, despite favorable changes in the lipid profile. These unfavorable outcomes were likely to be attributed to off-target effects, such as increase in blood pressure which is likely related to increased aldosterone secretion from adrenal glands (Hu et al., 2009, Endocrinology 150:2211-9; Forrest et al., 2008, British journal of pharmacology 154:1465-73). Other CETP inhibitors such as anacetrapib, dalcetrapib, and evacetrapib have been developed, which seem to lack the off-target effects of torcetrapib. These compounds do not affect aldosterone secretion. In the DEFINE (Determining the Efficacy and Tolerability of CETP Inhibition with Anacetrapib) trial, anacetrapib increases HDL-cholesterol by about 140% and lower LDL-cholesterol by 40% as compared to atorvastatin (Cannon et al., 2010, The New England journal of medicine 363:2406-15). An interim analysis of the dal-OUTCOMES trial, showed no benefit of dalcetrapib compared to placebo in ACS patients whereas HDL-cholesterol increase by about 30% and ApoA-I by 18% with no changes in LDL-cholesterol (Schwartz et al., 2012, The New England journal of medicine 121105113014000). The lack of efficacy was postulated to be related to the downregulation of ABCA1 by statins (Niesor et al. poster 167 presented at the American College of Cardiology, 62^{nd} annual scientific sessions Mar. 9-11, 2013, San Francisco, Calif., USA).

Fibrates are a class of lipid-lowering drugs used to treat various forms of hyperlipidemia (i.e., elevated serum triglycerides) that may also be associated with hypercholesterolemia. Fibrates appear to reduce the VLDL fraction and modestly increase HDL, however the effect of these drugs on serum cholesterol is variable. In the United States, fibrates such as clofibrate (Atromid-S®), fenofibrate (Tricor®) and bezafibrate (Bezalip®) have been approved for use as antilipidemic drugs, but have not received approval as hypercholesterolemia agents. For example, clofibrate is an antilipidemic agent that acts (via an unknown mechanism) to lower serum triglycerides by reducing the VLDL fraction. Although serum cholesterol may be reduced in certain patient subpopulations, the biochemical response to the drug is variable, and is not always possible to predict which patients will obtain favorable results. Atromid-S® has not been shown to be effective for prevention of coronary heart disease. The chemically and pharmacologically related drug, gemfibrozil (Lopid®) is a lipid regulating agent that moderately decreases serum triglycerides and VLDL cholesterol, and moderately increases HDL cholesterol—the HDL₂ and HDL₃ subfractions as well as both ApoA-I and A-II (i.e., the AI/AMT-HDL fraction). However, the lipid response is heterogeneous, especially among different patient populations. Moreover, while prevention of coronary heart disease was observed in male patients between 40-55 without history or symptoms of existing coronary heart disease, it is not clear to what extent these findings can be extrapolated to other patient populations (e.g., women, older and younger males). Indeed, no efficacy was observed in patients with established coronary heart disease. Serious side-effects are associated with the use of fibrates including toxicity such as malignancy (especially gastrointestinal cancer), gallbladder disease and an increased incidence in non-coronary mortality.

Oral estrogen replacement therapy may be considered for moderate hypercholesterolemia in post-menopausal women. However, increases in HDL may be accompanied with an increase in triglycerides. Estrogen treatment is, of course, limited to a specific patient population (postmenopausal women) and is associated with serious side effects including induction of malignant neoplasms, gall bladder disease, thromboembolic disease, hepatic adenoma, elevated blood pressure, glucose intolerance, and hypercalcemia.

Other agents useful for the treatment of hyperlipidemia include ezetimibe (Zetia®; Merck), which blocks or inhibits cholesterol absorption. However, inhibitors of ezetimibe have been shown to exhibit certain toxicities.

HDL, as well as recombinant forms of ApoA-I complexed with phospholipids can serve as sinks/scavengers for apolar or amphipathic molecules, e.g., cholesterol and derivatives (oxysterols, oxidized sterols, plant sterols, etc.), cholesterol esters, phospholipids and derivatives (oxidized phospholipids), triglycerides, oxidation products, and lipopolysaccharides (LPS) (see, e.g., Casas et al., 1995, J. Surg. Res. November 59(5):544-52). HDL can also serve as also a scavenger for TNF-alpha and other lymphokines. HDL can also serve as a carrier for human serum paraoxonases, e.g., PON-1,-2,-3. Paraoxonase, an esterase associated with HDL, is important for protecting cell components against oxidation. Oxidation of LDL, which occurs during oxidative stress, appears directly linked to development of atherosclerosis (Aviram, 2000, Free Radic. Res. 33 Suppl:S85-97). Paraoxonase appears to play a role in susceptibility to atherosclerosis and cardiovascular disease (Aviram, 1999, Mol. Med. Today 5(9):381-86). Human serum paraoxonase (PON-1) is bound to high-density lipoproteins (HDLs). Its activity is inversely related to atherosclerosis. PON-1 hydrolyzes organophosphates and may protect against atherosclerosis by inhibition of the oxidation of HDL and low-density lipoprotein (LDL) (Aviram, 1999, Mol. Med. Today 5(9):381-86). Experimental studies suggest that this protection is associated with the ability of PON-1 to hydrolyze specific lipid peroxides in oxidized lipoproteins. Interventions that preserve or enhance PON-1 activity may help to delay the onset of atherosclerosis and coronary heart disease.

HDL further has a role as an antithrombotic agent and fibrinogen reducer, and as an agent in hemorrhagic shock (Cockerill et al., WO 01/13939, published Mar. 1, 2001). HDL, and ApoA-I in particular, has been show to facilitate an exchange of lipopolysaccharide produced by sepsis into lipid particles comprising ApoA-I, resulting in the functional neutralization of the lipopolysaccharide (Wright et al., WO9534289, published Dec. 21, 1995; Wright et al., U.S. Pat. No. 5,928,624 issued Jul. 27, 1999; Wright et al., U.S. Pat. No. 5,932,536, issued Aug. 3, 1999).

Recently, different trials have described the difficulty to reduce coronary risk with Drugs increasing HDL-cholesterol, such as fibrates, niacin or inhibitors of CETP, beyond that achieved with statin therapy alone (see above). In several inborn errors of human HDL metabolism as well as on genetic mouse models with altered HDL metabolism, the changes in HDL-C were not associated with changes in cardiovascular risks or atherosclerotic plaque size respectively (Besler et al., 2012, EMBO molecular medicine 4:251-68; Voight et al., 2012, Lancet 6736:1-9; Frikke-Schmidt et al., JAMA, Jun. 4, 2008-Vol 299, No. 21; Holmes et al., Eur Heart J first published online Jan. 27, 2014 doi:10.1093/eurheartj/eht571). Thus, the pathogenic role and suitability of HDL as therapeutic target is questionable. This lead to the conclusion, that the functionality of the HDL rather than the simple HDL-cholesterol levels (as a biomarker) might be critical to evaluate in future clinical trials the benefit of the HDL in cardiovascular disease. When studying the functionality of the HDL it appears that the HDL metabolism is highly regulated and therefore one can hypothesis that extreme alterations such as strong increase in HDL levels (as achieved with CETP inhibitors therapy for instance) could drive to down-regulations, which would lead to modest impact on cardiovascular disease. This hypothesis is emphasized by results from the two clinical trials, which used different reconstituted HDL and where no dose-response relationship was observed. Moreover, a tendency to present less effect at the highest doses than the lower doses on plaque regression was described in both trials (Nissen et al., 2003, JAMA 290:2292-300; Tardif et al., 2007, JAMA 297:1675-82). The lack of beneficial effect of CETP inhibitor, Dalcetrapib in a recent clinical trial was further analyzed and lead to the conclusion that some statins could have specific down-regulation effect on ABCA1 expression in macrophages which could impaired the HDL benefit in atherosclerotic plaque regression in ACS patients. Altogether, those observations allow to conclude that the right increase of the HDL level or the nature of the HDL (pre-betan HDL versus spherical HDL), or the number of HDL particles, could be the key to successful treatment of cardiovascular disease.

HDL from healthy subjects can exert several protective effects in the vasculature and, in particular, on endothelial cells (Besler et al., 2011, The Journal of clinical investigation 121:2693-708; Yuhanna et al., 2001, Nature medicine 7:853-7; Kuvin et al., 2002, American heart journal 144:165-72). HDL from healthy subjects stimulates NO release from human aortic endothelial cells in culture and increases the expression of eNOS. (Besler et al., 2011, The Journal of clinical investigation 121:2693-708; Yuhanna et al., 2001, Nature medicine 7:853-7; Kuvin et al., 2002, American heart journal 144:165-72) HDL suppress the expression of adhesion molecules, such as vascular cell adhesion molecule 1 (VCAM1), and thus inhibits the adhesion of monocytes. (Nicholls et al., 2005, Circulation 111:1543-50; Ansell et al., 2003, Circulation 108:2751-6). HDL also exerts antithrombotic effects as described above. In a mouse carotid artery model, HDL enhances endothelial repair after vascular injury (Besler et al., 2011, The Journal of clinical investigation 121:2693-708). HDL obtained from healthy subjects reduced endothelial cell apoptosis in vitro and in apoE-deficient mice in vivo (Riwanto et al., 2013, Circulation 127:891-904). Such effects are observed also in patients with mutations in ABCA1 (Attie et al., 2001, J Lipid Res 42:1717-26). Infusion of reconstituted HDL particles (ApoA-1/phosphatidylcholine at a molar ratio of 1:150) improves impaired endothelial function as observed by intra-arterial infusion of acetylcholine and measurement of forearm blood flow by plethysmography or high-resolution ultrasound of the brachial artery and flow-mediated vasodilation, respectively (Spieker et al., 2002, Circulation 105:1399-402). In patients with, or at risk of, coronary heart disease (CHD) in a double-blind randomized placebo-controlled trial (dal-VESSEL), CETP inhibitor (Dalcetrapib) reduced CETP activity and increased HDL-C levels without affecting NO-dependent endothelial function, blood pressure, or markers of inflammation and oxidative stress (Lüscher et al., 2012, European heart journal 33:857-65). One hypothesis, is unlike HDL from healthy subjects, HDL from patients with diabetes mellitus, CAD, ACS, or chronic renal dysfunction are dysfunctional in the vascular effects as they no longer simulates NO release from endothelial cells in culture (Besler et al., 2011, The Journal of clinical investigation 121:2693-708; Sorrentino et al., 2010, Circulation 121:110-22; Speer et al., 2013, Immunity 1-15).

The therapeutic use of ApoA-I, ApoA-I_{M}, ApoA-I_{P} and other variants, as well as reconstituted HDL, is presently limited, however, by the large amount of apolipoprotein required for therapeutic administration and by the cost of protein production, considering the low overall yield of production and the occurrence of protein degradation in cultures of recombinantly expressed proteins. (See, e.g., Mallory et al., 1987, J. Biol. Chem. 262(9):4241-4247; Schmidt et al., 1997, Protein Expression & Purification 10:226-236). It has been suggested by early clinical trials that the dose range is between 1.5-4 g of protein per infusion for treatment of cardiovascular diseases. The number of infusions required for a full treatment is unknown. (See, e.g., Eriksson et al., 1999, Circulation 100(6):594-98; Carlson, 1995, Nutr. Metab. Cardiovasc. Dis. 5:85-91; Nanjee et al., 2000, Arterioscler. Thromb. Vasc. Biol. 20(9):2148-55; Nanjee et al., 1999, Arterioscler. Thromb. Vasc. Biol. 19(4):979-89; Nanjee et al., 1996, Arterioscler. Thromb. Vasc. Biol. 16(9):1203-14).

Recombinant human ApoA-I has been expressed in heterologous hosts, however, the yield of mature protein has been insufficient for large-scale therapeutic applications, especially when coupled to purification methods that further reduce yields and result in impure product.

Weinberg et al., 1988, J. Lipid Research 29:819-824, describes the separation of apolipoproteins A-I, A-II and A-IV and their isoforms purified from human plasma by reverse phase high pressure liquid chromatography.

WO 2009/025754 describes protein separation and purification of alpha-1-antitrypsin and ApoA-I from human plasma.

Hunter et al., 2009, Biotechnol. Prog. 25(2):446-453, describes large-scale purification of the ApoA-I Milano variant that is recombinantly expressed in E. coli.

Caparon et al., 2009, Biotechnol. And Bioeng. 105(2):239-249 describes the expression and purification of ApoA-I Milano from an E. coli host which was genetically engineered to delete two host cell proteins in order to reduce the levels of these proteins in the purified apolipoprotein product.

U.S. Pat. No. 6,090,921 describes purification of ApoA-I or apolipoprotein E (ApoE) from a fraction of human plasma containing ApoA-I and ApoE using anion-exchange chromatography.

Brewer et al., 1986, Meth. Enzymol. 128:223-246 describes the isolation and characterization of apolipoproteins from human blood using chromatographic techniques.

Weisweiler et al., 1987, Clinica Chimica Acta 169:249-254 describes isolation of ApoA-I and ApoA-II from human HDL using fast-protein liquid chromatography.

deSilva et al., 1990, J. Biol. Chem. 265(24):14292-14297 describes the purification of apolipoprotein J by immunoaffinity chromatography and reverse phase high performance liquid chromatography.

Lipoproteins and lipoprotein complexes are currently being developed for clinical use, with clinical studies using different lipoprotein-based agents establishing the feasibility of lipoprotein therapy (Tardif, 2010, Journal of Clinical Lipidology 4:399-404). One study evaluated autologous delipidated HDL (Waksman et al., 2010, J Am. Coll. Cardiol. 55:2727-2735). Another study evaluated ETC-216, a complex of recombinant ApoA-I_{M} and palmitoyl-oleoyl-PC (POPC) (Nissen et al., 2003, JAMA 290:2292-2300). CSL-111 is a reconstituted human ApoA-I purified from plasma complexed with soybean phosphatidylcholine (SBPC) (Tardif et al., 2007, JAMA 297:1675-1682). Current exploratory drugs have shown efficacy in reducing the atherosclerotic plaque but the effect was accompanied by secondary effects such as increase in transaminases or formation of ApoA-I antibodies (Nanjee et al., 1999, Arterioscler. Vasc. Throm. Biol. 19:979-89; Nissen et al., 2003, JAMA 290:2292-2300; Spieker et al., 2002, Circulation 105:1399-1402; Nieuwdorp et al., 2004, Diabetologia 51:1081-4; Drew et al., 2009, Circulation 119, 2103-11; Shaw et al., 2008, Circ. Res. 103:1084-91; Tardiff et al., 2007, JAMA 297:1675-1682; Waksman, 2008, Circulation 118:S 371; Cho, U.S. Pat. No. 7,273,849 B2, issued Sep. 25, 2007). For example, the ERASE clinical trial (Tardiff et al., 2007, JAMA 297:1675-1682) utilized two doses of CSL-111: 40 mg/kg and 80 mg/kg of ApoA-I. The 80 mg/kg dose group had to be stopped due to liver toxicity (as shown by serious transaminase elevation). Even in the 40 mg/kg dose group several patients experience transaminase elevation. Toxicity is potentially attributed to the presence of remaining cholate, the detergent used for the manufacturing of the reconstituted HDL (as highlighted by Wright et al., US 2013/0190226).

A need therefore exists for dosing regimens of cholesterol lowering drugs that are more effective in lowering serum cholesterol, increasing HDL serum levels, preventing and/or treating dyslipidemia and/or diseases, conditions and/or disorders associated with dyslipidemia yet minimize side effects such as liver toxicity and increases in triglycerides, LDL-triglycerides, or VLDL-triglycerides, as well as methods for identifying such dosing regimens and monitoring subjects receiving such treatment.

### 4. SUMMARY

In this era of personalized medicine, pharmacogenomics combining the science of drugs and genomics) have promoted the use and interrogation of so-called “companion diagnostics,” which are diagnostic products intended for use in conjunction with a therapeutic product to better inform treatment selection, initiation, dose customization, or avoidance. The present disclosure relates, in part, on the discovery of an inverted U-shaped dose-effect curve in response to treatment of subjects with HDL Therapeutics (as defined in Section 6.1 below), particularly HDL mimetics, delipidated or lipid poor HDLs, or other compounds that increase HDL levels following administration, via a mechanism of action that downregulates components of cholesterol efflux and reverse lipid transport, such as the ABCA1 and ABCG1 transporters and SREBP1, a transcription factor that regulates the biosynthesis of fatty acids. The discovery of this mechanism of action permits the design of companion diagnostic assays that are useful for monitor treatment with HDL Therapeutics and/or to identify an effective dosage of an HDL Therapeutic for a particular subject or sub-group or other group of subjects. Thus, the present disclosure relates, among other things, to HDL Marker companion diagnostic assays that can be used in concert with subjects receiving treatment with an HDL Therapeutic. In some embodiments, the present disclosure relates to methods for determining whether a subject receiving treatment with an HDL Therapeutic is receiving a therapeutically effective or optimal dose. In some embodiments, the present disclosure relates to methods for determining whether a subject receiving treatment with an HDL Therapeutic is receiving a therapeutically effective or optimal dose while optimizing the safety.

The methods as described herein can be employed wherein the subject is being treated for a Condition (as defined in Section 6.1 below) with an HDL Therapeutic, or to identify or optimize a dosing schedule for an HDL Therapeutic to treat a subject suffering from a Condition.

Also provided herein is a method of predicting the likelihood of response of a subject to treatment with an HDL Therapeutic.

In certain aspects, the present disclosure relates to methods of treating a subject suffering from a Condition with an HDL Therapeutic, identifying a suitable dose of an HDL Therapeutic for treating a Condition, mobilizing cholesterol in a subject suffering from a Condition, or monitoring the efficacy of an HDL Therapeutic in a subject. The methods typically comprise administering an HDL Therapeutic to a subject (one or more times, for example in accordance with a dosing regimen) and monitoring changes in gene expression of at least one, in some embodiments two or three or more, HDL Markers in a test sample from the individual. Any changes can be as compared to the subject's own baseline, the subject's prior measurements, and/or a control obtained from measuring the one or more HDL Markers in a population of individuals. The population of individuals can be any appropriate population, e.g., healthy individuals, individuals suffering from a Condition, genetically matched individuals, etc. Following measurement, the dose, frequency of dosing or both, can be adjusted if the HDL Therapeutic down regulates components of the cholesterol efflux pathway to a degree such that therapeutic efficacy is attenuated. In some embodiments, a dose is identified that does not alter or even increases the expression levels of one or more HDL Markers in the subject's circulating monocytes, macrophages or mononuclear cells.

In some embodiments, the methods comprise the steps of: (a) obtaining a first test sample from the subject or a population of subjects; (b) measuring expression levels of one or more HDL Markers (as defined in Section 6.1 below) in the test sample; (c) administering a dose (or a series of doses) of an HDL Therapeutic to the subject or a population of subjects; (d) obtaining a second test sample from the subject or the population of subjects; and (e) measuring expression levels of the one or more HDL Markers in the second test sample. In some embodiments, the first sample is obtained prior to treatment with the HDL Therapeutic. In other embodiments, the first sample is obtained after the subject or population of subjects is treated with a different dose of the HDL Therapeutic than the dose of step (c).

In other embodiments, the methods comprise the steps of: (a) administering a dose of an HDL Therapeutic to a subject or population of subject; (b) obtaining a test sample from the subject or the population of subjects; and (c) measuring expression levels of one or more HDL Markers in the test sample to determine the expression levels are above or below a cutoff amount. Optionally, steps (a) through (c) are repeated for one or more additional doses of the HDL Therapeutic until a suitable dose is identified. The additional doses can include higher/lower amounts of the HDL Therapeutics, higher/lower dosing frequency, or faster/slower infusion times.

The test sample is preferably a sample of peripheral blood mononuclear cells or circulating monocytes or macrophages. It could also be a sample of lymph mononuclear cells or circulating monocytes or macrophages. Samples can be obtained, e.g., from an untreated subject or population of subjects or from a subject or population of subjects following administration of the HDL Therapeutic, e.g., 2, 4, 6, 8, 10, 12, 16, 20 or 24 hours following administration. In varying embodiments, sample are obtained 2-10, 2-12, 4-6, 4-8, 4-24, 4-16, 6-8 or 6-10 hours after administration. The subjects can be treated with the HDL Therapeutic as a monotherapy or a part of a combination therapy regimen with, e.g., one or more lipid control medications such as atorvastatin, ezetimibe, niacin, rosuvastatin, simvastatin, aspirin, fluvastatin, lovastatin, and pravastatin. In some embodiments, identifying a suitable dose is carried out in healthy individuals and in other embodiments it is carried out in a population of individuals suffering from a Condition. In various embodiments, the suitable dose is a dose that reduces expression levels of one or more HDL Markers by 20%-80%, 30%-70%, 40%-60%, or 50% as compared to the subject's baseline amount and/or a population average. In other embodiments, the suitable dose is a dose that reduces expression levels of one or more HDL Markers by no more than 50%, and in some embodiments no more than 40%, no more than 30%, no more than 20%, or no more than 10% as compared to the subject's baseline amount or the population average. In yet other embodiments, the dose is one that does not reduce expression levels of one or more HDL Markers at all as compared to the subject's baseline amount or the population average.

In still another embodiment, provided herein is a kit for use in the companion diagnostic assays of the disclosure. In some embodiments, the kit comprises (a) at least one HDL Therapeutic and (b) at least one diagnostic reagent useful for quantitating expression of an HDL Marker (e.g., primers and/or probes for detection of an HDL Marker in the case of a nucleic acid assay and at least one anti-HDL Marker antibody (polyclonal or monoclonal) in the case of a protein assay). In another embodiment, HDL markers are determined with the help of a cell sorter or a FACS instrument used to separate cells from a biological sample (for instance blood or lymph).

Also presented herein are methods of treating a subject suffering from familial hypoalphalipoproteinemia, e.g., an ABCA1 deficiency, with an HDL Therapeutic. Preferably, the therapy is given in two phases, an initial, more intense “induction” phase and a subsequent, less intense “maintenance” phase. Optionally, the therapy is given according to a dosing schedule identified using the methods described herein.

Also presented herein are methods of treating a subject suffering from an LCAT deficiency (homozygote or heterozygote) with an HDL Therapeutic, optionally using a dosing schedule identified using the methods described herein.

Also presented herein are methods of treating a subject suffering from an ApoA-I deficiency (homozygote or heterozygote) with an HDL Therapeutic, optionally using a dosing schedule identified using the methods described herein.

Also presented herein are methods of treating a subject suffering from an low HDL levels (below 40 mg/dl of HDL-chol in men or below 50 mg/dl of HDL-chol in women) with an HDL Therapeutic, optionally using a dosing schedule identified using the methods described herein.

In certain embodiments, the disclosure provides a method of identifying a dose of an HDL Therapeutic effective to mobilize cholesterol in a subject. In some embodiments, the method comprises: (a) administering a first dose of an HDL Therapeutic to a subject, (b) following administering said first dose, measuring expression levels of one or more HDL Markers in said subject's circulating monocytes, macrophages or mononuclear cells to evaluate the effect of said first dose on said expression levels; and (c) (i) if the subject's expression levels of one or more HDL Markers are reduced by more than a cutoff amount, administering a second dose of said HDL Therapeutic, wherein the second dose of said HDL Therapeutic is lower than the first dose; or (ii) if the subject's expression levels of one or more HDL Markers are not reduced by more than the cutoff amount, treating the subject with the first dose of said HDL Therapeutic.

In certain embodiments, the disclosure provides a method for monitoring the efficacy of an HDL Therapeutic in a subject. In some embodiments, the method comprises: (a) treating a subject with an HDL Therapeutic according to a first dosing schedule, (b) measuring expression levels of one or more HDL Markers in said subject's circulating monocytes, macrophages or mononuclear cells to evaluate the effect of said first dosing schedule on said expression levels; and (c) (i) if the subject's expression levels of one or more HDL Markers are reduced by more than an upper cutoff amount, treating the subject with the HDL Therapeutic according to a second dosing schedule, wherein the second dosing schedule comprises one or more of: administering a lower dose of the HDL Therapeutic, infusing the HDL Therapeutic into the subject over a longer period of time, and administering the HDL Therapeutic to the subject on a less frequent basis; (ii) if the subject's expression levels of one or more HDL Markers are not reduced by more than a lower cutoff amount, treating the subject with the HDL Therapeutic according to a second dosing schedule, wherein the second dosing schedule comprises one or more of: administering a higher dose of the HDL Therapeutic, infusing the HDL Therapeutic into the subject over a shorter period of time, and administering the HDL Therapeutic to the subject on a more frequent basis; or (iii) if the subject's expression levels of one or more HDL Markers are reduced by an amount between the upper and lower cutoff amounts, continuing to treat the subject according to the first dosing schedule.

The cutoff amount may be relative to the subject's own baseline prior to said administration or the cutoff amount may be relative to a control amount such as a population average from e.g., healthy subjects or a population with the same disease condition as the subject or a population sharing one more disease risk genes with the subject.

In certain embodiments, the disclosure provides a method of identifying a dose of an HDL Therapeutic effective to mobilize cholesterol. In some embodiments, the method comprises: (a) administering a first dose of an HDL Therapeutic to a population of subjects; (b) following administering said first dose, measuring expression levels of one or more HDL Markers in said subjects' circulating monocytes, macrophages or mononuclear cells to evaluate the effect of said first dose on said expression levels; (c) administering a second dose of said HDL Therapeutic, wherein the second dose of said HDL Therapeutic is greater or lower than the first dose; (d) following administering said second dose, measuring expression levels of one or more HDL Markers in said subjects' circulating monocytes, macrophages or mononuclear cells to evaluate the effect of said first dose on said expression levels; (e) optionally repeating steps (c) and (d) with one or more additional doses of said HDL Therapeutic; and (f) identifying the highest dose that does not reduce expression levels of one or more HDL Markers in by more than a cutoff amount, thereby identifying a dose of said HDL Therapeutic effective to mobilize cholesterol.

In certain embodiments, following administration of said second dose, expression levels of one or more HDL Markers in said subject's circulating monocytes, macrophages or mononuclear cells is measured to evaluate the effect of said second dose on said expression levels. If the subject's expression levels of one or more HDL Markers are reduced by more than a cutoff amount, a third dose of said HDL Therapeutic may be administered, wherein the third dose of said HDL Therapeutic is lower than the second dose.

In certain embodiments, the disclosure provides a method for treating a subject in need of an HDL Therapeutic. In some embodiments, the method comprises administering to subject a combination of: (a) a lipoprotein complex in a dose that does not reduce expression of one or more HDL Markers in said subject's circulating monocytes, macrophages or mononuclear cells by more than 20% or more than 10% as compared to the subject's baseline amount; and (b) a cholesterol reducing therapy, optionally selected from a bile-acid resin, niacin, a statin, a fibrate, a PCSK9 inhibitor, ezetimibe, and a CETP inhibitor.

In certain embodiments, the disclosure provides a method for treating a subject in need of an HDL Therapeutic. In some embodiments, the method comprises administering to subject a combination of: (a) a lipoprotein complex in a dose that does not reduce expression of one or more HDL Markers in said subject's circulating monocytes, macrophages or mononuclear cells by more than 20% or more than 10% as compared to a control amount; and (b) a cholesterol reducing therapy, optionally selected from a bile-acid resin, niacin, a statin, a fibrate, a PCSK9 inhibitor, ezetimibe, and a CETP inhibitor.

The control amount may be the population average, e.g., the population average from healthy subjects or a population with the same disease condition as the subject or a population sharing one more disease risk genes with the subject. The subject may be human or the population of subjects is a population of human subjects. The subject may be a non-human animal, e.g., mouse, or the population of subjects may be a population of non-human animals.

In certain embodiments of the methods described herein, at least one HDL Marker is ABCA1. For example, ABCA1 mRNA expression levels or ABCA1 protein expression levels are measured. In various embodiments, the ABCA1 cutoff amount is 10%, 20%, 30%, 40%, 50%, 60%, 70% or 80%, or selected from any range bounded by any two of the foregoing cutoff amounts, e.g., 20%-80%, 30%-70%, 40%-60%, 10%-50%, 10%-40%, 20%-50%, and so on and so forth. ABCA1 expression levels may be measured 2-12 hours, 4-10 hours, 2-8 hours, 2-6 hours, 4-6 hours or 4-8 hours after administration.

In certain embodiments of the methods described herein, at least one HDL Marker is ABCG1. For example, ABCG1 mRNA expression levels or ABCG1 protein expression levels are measured. In various embodiments, the ABCG1 cutoff amount is 10%, 20%, 30%, 40%, 50%, 60%, 70% or 80%, or selected from any range bounded by any two of the foregoing cutoff amounts, e.g., 20%-80%, 30%-70%, 40%-60%, 10%-50%, 10%-40%, 20%-50%, and so on and so forth. ABCG1 expression levels may be measured 2-12 hours, 4-10 hours, 2-8 hours, 2-6 hours, 4-6 hours or 4-8 hours after administration.

In certain embodiments of the methods described herein, at least one HDL Marker is SREBP-1. For example, SREBP-1 mRNA expression levels or SREBP-1 protein expression levels are measured. In various embodiments, the SREBP1 cutoff amount is 10%, 20%, 30%, 40%, 50%, 60%, 70% or 80%, or selected from any range bounded by any two of the foregoing cutoff amounts, e.g., 20%-80%, 30%-70%, 40%-60%, 10%-50%, 10%-40%, 20%-50%, and so on and so forth. SREBP-1 expression levels may be measured 2-12 hours, 4-10 hours, 2-8 hours, 2-6 hours, 4-6 hours or 4-8 hours after administration.

In certain embodiment, the HDL Therapeutic is a lipoprotein complex. The lipoprotein complex may comprise an apolipoprotein such as ApoA-I, ApoA-II, ApoA-IV, ApoE or a combination thereof. The lipoprotein complex may comprise an apolipoprotein peptide mimic such as an ApoA-I, ApoA-II, ApoA-IV, or ApoE peptide mimic or a combination thereof. The lipoprotein complex may be CER-001, CSL-111, CSL-112, CER-522, or ETC-216.

In certain embodiments, the HDL Therapeutic is a small molecule such as a CETP inhibitor or a pantothenic acid derivative.

In certain embodiments, the methods described herein further comprise determining a cutoff amount. For example, the cutoff amount may be determined by generating a dose response curve for the HDL Therapeutic. The cutoff amount may be 25%, 40%, 50%, 60% or 75% of the expression level of the HDL Marker at the inflection point in the dose response curve. In particular embodiments, the cutoff is selected from a range bounded by any two of the foregoing cutoff values, e.g., 30%-70%, 40%-60%, 25%-50%, 25%-75% of the expression level of the HDL Marker at the inflection point in the dose response curve.

In certain embodiments, the subject or population of subjects has an ABCA1 deficiency. The subject or population of subjects may be homozygous for an ABCA1 mutation. The subject or population of subjects may be heterozygous for an ABCA1 mutation.

In other embodiments, the subject or population of subjects has an HDL deficiency, hypoalphalipoproteinemia, or primary familial hypoalphalipoproteinemia.

In other embodiments, the subject or population of subjects has an LCAT deficiency or Fish-eye disease. The subject or population of subjects may be homozygous for an LCAT mutation. The subject or population of subjects may be heterozygous for an LCAT mutation.

In other embodiments, the subject or population of subjects has an ABCG1 deficiency. The subject or population of subjects may be homozygous for an ABCG1 mutation. The subject or population of subjects may be heterozygous for an ABCG1 mutation.

In other embodiments, the subject or population of subjects has an ApoA-I deficiency. The subject or population of subjects may be homozygous for an ApoA-I mutation. The subject or population of subjects may be heterozygous for an ApoA-I mutation.

In yet other embodiments, the subject or population of subjects has an ABCG8 deficiency. The subject or population of subjects may be homozygous for an ABCG8 mutation. The subject or population of subjects may be heterozygous for an ABCG8 mutation.

In yet other embodiments, the subject or population of subjects has a PLTP deficiency. The subject or population of subjects may be homozygous for a PLTP mutation. The subject or population of subjects may be heterozygous for a PLTP mutation.

The patient can have genetic defects in one or more of the foregoing genes, i.e., has compounded genetic defects.

In certain embodiments, the disclosure provides a method of identifying a dose of an HDL Therapeutic suitable for therapy. In some embodiments, the method comprises: (a) administering one or more doses of an HDL Therapeutic to a subject; (b) measuring expression levels of one or more HDL Markers in said subject's circulating monocytes, macrophages or mononuclear cells following each dose; and (c) identifying the maximum dose that does not reduce expression levels of said one or more HDL Markers by more than 0%, more than 10% or more than 20%, thereby identifying a dose of an HDL Therapeutic suitable for therapy.

In other embodiments, the disclosure provides a method of identifying a dose of an HDL Therapeutic suitable for therapy. In some embodiments, the method comprises: (a) administering one or more doses of an HDL Therapeutic to a subject; (b) measuring expression levels of one or more HDL Markers in said subject's circulating monocytes, macrophages or mononuclear cells following each dose; and (c) identifying a dose that maintains baseline expression levels or even raises the expression levels of one or more HDL Markers in the subject's circulating monocytes, macrophages or mononuclear cells, thereby identifying a dose of an HDL Therapeutic suitable for therapy. The levels can be increased by at least 10%, at least 20%, at least 30%, at least 40%, at least 50%, at least 60%, at least 70%, at least 80%, or in a range bounded by any two of the foregoing values, e.g., the levels can be increased by up to 10%, up to 20%, up to 50%, 10%-50%, 20%-60%, and so on and so forth.

In certain embodiments, the disclosure provides a method of identifying a dose of an HDL Therapeutic suitable for therapy. In some embodiments, the method comprises: (a) administering one or more doses of an HDL Therapeutic to a population of subjects; (b) measuring expression levels of one or more HDL Markers in said subjects' circulating monocytes, macrophages or mononuclear cells following each dose; and (c) identifying the maximum dose that does not raise expression levels of said one or more HDL Markers by more than 0%, more than 10% or more than 20% in said subjects, thereby identifying a dose of an HDL Therapeutic suitable for therapy.

In certain embodiments, the disclosure provides a method of identifying a dose of an HDL Therapeutic suitable for therapy. In some embodiments, the method comprises: (a) administering one or more doses of an HDL Therapeutic to a population of subjects; (b) measuring expression levels of one or more HDL Markers in said subjects' circulating monocytes, macrophages or mononuclear cells following each dose; and (c) identifying a dose that maintain baseline expression levels or even raises the expression levels of one or more HDL Markers in the subject's circulating monocytes, macrophages or mononuclear cells, thereby identifying a dose of an HDL Therapeutic suitable for therapy. The levels can be increased by at least 10%, at least 20%, at least 30%, at least 40%, at least 50%, at least 60%, at least 70%, at least 80%, or in a range bounded by any two of the foregoing values, e.g., the levels can be increased by up to 10%, up to 20%, up to 50%, 10%-50%, 20%-60%, and so on and so forth.

In certain embodiments, the disclosure provides a method of identifying a dose of an HDL Therapeutic suitable for therapy. In some embodiments, the method comprises identifying the highest dose of the HDL therapeutic that does not reduce cellular cholesterol efflux by more than 0%, more than 10% or more than 20%. A method of identifying a dose of an HDL Therapeutic suitable for therapy may comprise: (a) administering one or more doses of an HDL Therapeutic to a subject or population of subjects; (b) measuring cholesterol efflux in cells from said subject or population of subjects; and (c) identifying the maximum dose that does not reduce cholesterol efflux by more than 0%, more than 10% or more than 20% in said subjects, thereby identifying a dose of an HDL Therapeutic suitable for therapy.

In certain embodiments, the disclosure provides a method of identifying a dosing interval of an HDL Therapeutic suitable for therapy. In some embodiments, the method comprises identifying the highest dose of the most frequent dosing regimen of the HDL therapeutic that does not reduce cellular cholesterol efflux by more than 0%, more than 10% or more than 20%. A method of identifying a dosing interval of an HDL Therapeutic suitable for therapy may comprise: (a) administering an HDL Therapeutic to a subject or population of subjects according to one or more dosing frequencies; (b) measuring cholesterol efflux in cells from said subject or population of subjects; and (c) identifying the maximum dosing frequency that does not reduce cholesterol efflux by more than 50% to 100% in said subjects, thereby identifying a dose of an HDL Therapeutic suitable for therapy.

In certain embodiments, the one or more dosing frequencies includes one or more dosing frequencies selected from: (a) administration as a 1-4 hour infusion every 2 days; (b) administration as a 1-4 hour an infusion every 3 days; (c) administration as a 24 hour infusion every week day; and (d) administration as a 24 hour infusion every two weeks.

Cholesterol efflux may be measured in monocytes, macrophages or mononuclear cells from said subjects or populations of subjects.

In certain embodiments, the disclosure provides a method for treating a subject with an ABCA1 deficiency. In some embodiments, the method comprises administering to the subject a therapeutically effective amount of an HDL Therapeutic such as CER-001. The subject may be heterozygous or homozygous for an ABCA1 mutation.

In certain embodiments, the disclosure comprises a method of treating a subject suffering from familial primary hypoalphalipoproteinemia. In some embodiments, the method comprises: (a) administering to the subject an HDL Therapeutic according to an induction regimen; and, subsequently (b) administering to the subject the HDL Therapeutic according to a maintenance regimen. The maintenance regimen may entail administering the HDL therapeutic at a lower dose, a lower frequency, or both. The subject may be heterozygous or homozygous for an ABCA1 mutation. The subject may be homozygous or heterozygous for an LCAT mutation. The subject may be homozygous or heterozygous for an ApoA-I mutation. The subject may be homozygous or heterozygous for an ABCG1 mutation. The subject may also be treated with a lipid control medication such as atorvastatin, ezetimibe, niacin, rosuvastatin, simvastatin, aspirin, fluvastatin, lovastatin, pravastatin or a combination thereof.

The HDL Therapeutic may be CER-001 and/or the induction regimen may be for a duration of 4 weeks. The induction regimen may comprise administering the HDL Therapeutic two, three or four times a week. Where the HDL Therapeutic is a lipoprotein complex such as CER-001, the dose administered in the induction regimen can be selected from 8-15 mg/kg (on a protein weight basis). In particular embodiments, the induction dose is 8 mg/kg, 12 mg/kg or 15 mg/kg. The maintenance regimen may comprise administering the HDL Therapeutic for at least one month, at least two months, at least three months, at least six months, at least a year, at least 18 months, at least two years, or indefinitely. The maintenance regimen may comprise administering the HDL Therapeutic once or twice a week. Where the HDL Therapeutic is a lipoprotein complex such as CER-001, the dose administered in the maintenance regimen can be selected from 1-6 mg/kg (on a protein weight basis). In particular embodiments, the maintenance dose is 1 mg/kg, 3 mg/kg or 6 mg/kg.

In certain embodiments, (a) the induction regimen utilizes a dose that reduces expression levels of one or more HDL Markers by 20%-80% or 40%-60%, as compared to the subject's baseline amount and/or a population average; and/or (b) wherein the maintenance regimen utilizes a dose that does not reduce expression levels of one or more HDL Markers by more than 20% or more than 10% as compared to the subject's baseline amount and/or a population average.

It should be noted that the indefinite articles “a” and “an” and the definite article “the” are used in the present application, as is common in patent applications, to mean one or more unless the context clearly dictates otherwise. Further, the term “or” is used in the present application, as is common in patent applications, to mean the disjunctive “or” or the conjunctive “and.”

The features and advantages of the disclosure will become further apparent from the following detailed description of embodiments thereof.

### 5. BRIEF DESCRIPTION OF THE FIGURES

FIG. 1 depicts the CHI SQUARE study design;

FIGS. 2A-2C depict ApoA-1, phospholipid and total plasma concentrations following administration of the first and sixth infusions of CER-001;

FIG. 3 depicts distribution of frames between MHICC and SAHMRI;

FIG. 4 depicts LS mean change in TAV and PAV-mITT population;

FIG. 5 depicts LS mean change in TAV and PAV-mPP population;

FIGS. 6A-6B depict an inverted U-shaped dose-effect curve of CER-001.

FIG. 7 depicts the effect of CER-001, HDL₃ or ApoA-I on ABCA1 expression in J774 macrophages;

FIG. 8 depicts the effect of CER-001, HDL₃ or ApoA-I on ABCG1 expression in J774 macrophages;

FIG. 9 depicts the effect of CER-001, HDL₃ or ApoA-I on SR-BI expression in J774 macrophages;

FIG. 10 depicts the effect of CER-001, HDL₃ or ApoA-I on SREBP-1 expression in J774 macrophages;

FIG. 11 depicts the effect of CER-001, HDL₃ or ApoA-I on SREBP-2 expression in J774 macrophages;

FIG. 12 depicts the effect of CER-001, HDL₃ or ApoA-I on LXR expression in J774 macrophages;

FIG. 13 depicts the expression in J774 macrophages of ABCA1 treated with doses (μg/mL) of CER-001, HDL₃ or ApoA-I;

FIG. 14 depicts the expression in J774 macrophages of ABCG1 treated with doses (μg/mL) of CER-001, HDL₃ or ApoA-I;

FIG. 15 depicts the expression in J774 macrophages of SREBP-1 treated with doses (μg/mL) of CER-001, HDL₃ or ApoA-I;

FIG. 16 depicts the expression in J774 macrophages of SR-BI treated with doses (μg/mL) of CER-001, HDL₃ or ApoA-I;

FIG. 17 depicts the decreasing mRNA levels of ABCA1 over time after J774 macrophages are treated with CER-001, HDL₃ or ApoA-I;

FIG. 18 depicts ABCA1 mRNA levels in J774 macrophages in the presence and absence of cAMP;

FIG. 19 depicts ABCG1 mRNA levels in J774 macrophages in the presence and absence of cAMP;

FIG. 20 depicts the effect of CER-001 and HDL₃ on ABCA1 protein level in J774 macrophages;

FIG. 21 depicts the effect of CER-001 and HDL₃ on ABCA1 protein level in J774 macrophages;

FIG. 22 depicts the effect of cAMP on the regulation of ABCA1 mRNA levels in J774 macrophages in the presence of increasing concentrations of CER-001;

FIG. 23 depicts the effect set to zero of cAMP on the regulation of ABCA1 mRNA levels in J774 macrophages in the presence of increasing concentrations of CER-001;

FIG. 24 depicts the effect of cAMP on the regulation of ABCG1 mRNA levels in J774 macrophages in the presence of increasing concentrations of CER-001;

FIG. 25 depicts the effect set to zero of cAMP on the regulation of ABCG1 mRNA levels in J774 macrophages in the presence of increasing concentrations of CER-001;

FIG. 26 depicts the effect of cAMP on the regulation of ABCA1 mRNA levels in J774 macrophages in the presence of increasing concentrations of CER-001;

FIG. 27 depicts the time necessary to return to the baseline amount of ABCA1 after treatment with CER-001, HDL₃, and ApoA-I;

FIG. 28 depicts the time necessary to return to the baseline amount of ABCG1 after treatment with CER-001, HDL₃, and ApoA-I;

FIG. 29 depicts the time necessary to return to the baseline amount of SR-BI after treatment with CER-001, HDL₃, and ApoA-I;

FIG. 30 depicts the effect of CER-001, HDL₃ and ApoA-I on ABCA1 levels in HepG2 hepatocytes;

FIG. 31 depicts the effect of CER-001, HDL₃ and ApoA-I on SR-BI levels in HepG2 hepatocytes;

FIG. 32 depicts the effect of CER-001, HDL₃ and ApoA-I on ABCA1 levels in Hepa 1.6 hepatocytes;

FIG. 33 depicts the effect of CER-001, HDL₃ and ApoA-I on SR-BI levels in Hepa 1.6 hepatocytes;

FIG. 34 depicts the effect of ApoA-1 addition after ABCA1 down-regulation by CER-001 and HDL₃;

FIG. 35 depicts the effect of ApoA-1 addition after ABCG1 down-regulation by CER-001 and HDL₃;

FIG. 36 depicts the effect of ApoA-1 addition after SR-BI down-regulation by CER-001 and HDL₃;

FIG. 37 depicts the effect of HDL₂ on ABCA1 mRNA levels in J774 macrophages;

FIG. 38 depicts the effect of HDL₂ on ABCG1 mRNA levels in J774 macrophages;

FIG. 39 depicts the effect of HDL₂ on SR-BI mRNA levels in J774 macrophages;

FIG. 40 depicts the effect of β-cyclodextrin on cholesterol efflux;

FIG. 41 depicts a dose-dependent decrease for ABCA1 mRNA levels in J774 macrophages in the presence of β-cyclodextrin;

FIG. 42 depicts a dose-dependent decrease for ABCG1 mRNA levels in J774 macrophages in the presence of β-cyclodextrin;

FIG. 44 depicts a dose-dependent increase for SR-BI mRNA levels in J774 macrophages in the presence of β-cyclodextrin;

FIG. 44 depicts the effect of β-cyclodextrin on LXR mRNA levels in J774 macrophages;

FIG. 45 depicts the effect of β-cyclodextrin on SREBP1 mRNA levels in J774 macrophages;

FIG. 46 depicts the effect of β-cyclodextrin on SREBP2 mRNA levels in J774 macrophages;

FIG. 47 depicts the unesterified cholesterol content in ligatured carotids for mice treated with CER-001 and HDL₃;

FIG. 48 depicts the total cholesterol content in ligatured carotids for mice treated with CER-001 and HDL₃;

FIG. 49 depicts the plasma total cholesterol levels after CER-001 infusion;

FIG. 50 depicts the plasma total cholesterol levels after HDL₃ infusion;

FIG. 51 depicts the plasma unesterified cholesterol levels after CER-001 infusion;

FIG. 52 depicts the plasma unesterified cholesterol levels after HDL₃ infusion;

FIG. 53 depicts the post-dose plasma total cholesterol levels for CER-001 and HDL₃;

FIG. 54 depicts the post-dose plasma unesterified cholesterol levels for CER-001 and HDL₃;

FIG. 55 depicts the plasma ApoA-I levels following dosage with CER-001;

FIG. 56 depicts the plasma ApoA-I levels following dosage with HDL₃;

FIG. 57 depicts western blot determination of ABCA1 expression in ligatured carotids;

FIG. 58 depicts the ABCA1 level in the liver 24 hours after the last injection of CER-001;

FIG. 59 depicts the SR-BI level in the liver 24 hours after the last injection of CER-001;

FIG. 60 depicts the cholesterol content measured in feces of mice injected with CER-001 and HDL₃.

FIG. 61 depicts an overview of HDL particle development;

FIG. 62 depicts an overview of the Reverse Lipid Transport (RLT) pathway;

FIG. 63 depicts an overview of HDL maturation steps;

FIG. 64 depicts the amino acid sequence of human ApoA-I (SEQ ID NO: 1);

FIGS. 65A1-65A3 and FIG. 65B depict the nucleotide and polypeptide sequences, respectively, of human ABCA1 (SEQ ID NOS 2 and 3, respectively);

FIGS. 66A1-66A2 and FIG. 66B depict the nucleotide and polypeptide sequences, respectively, of human ABCG1 (SEQ ID NOS 4 and 5, respectively);

FIGS. 67A1-67A2 and FIG. 67B depict the nucleotide and polypeptide sequences, respectively, of human SREBP1 (SEQ ID NOS 6 and 7, respectively).

FIGS. 68A-68G depict timecourse of cholesterol esterifcation in subjects in SAMBA clinical trial;

FIGS. 69A-69G depict esterification of loaded cholesterol by LCAT in subjects in SAMBA clinical trial;

FIG. 70 depicts carotid vessel wall thickness changes in individual subjects in SAMBA clinical trial after one month;

FIG. 71 depicts aortic vessel wall thickness changes in individual subjects in SAMBA clinical trial after one month; and

FIG. 72 depicts mean vessel wall thickness changes in SAMBA subject after one and six months.

### 6. DETAILED DESCRIPTION

### 6.1. Definitions

As used herein, the following terms are intended to have the following meanings:

Condition or Conditions means one, more or all of: dyslipidemic disorders (such as hyperlipidemia, hypercholesterolemia, coronary heart disease, coronary artery disease, vascular and perivascular diseases, and cardiovascular diseases such as atherosclerosis) and diseases associated with dyslipidemia (such as coronary heart disease, coronary artery disease, acute coronary syndrome, unstable angina pectoris, myocardial infarction, stroke, transient ischemic attack (TIA), endothelial dysfunction, thrombosis such as atherothrombotic vascular disease, inflammatory disease such as vascular endothelial inflammation, cardiovascular disease, hypertension, hypoxia-induced angiogenesis, apoptosis of endothelial cells, macular degeneration, type I diabetes, type II diabetes mellitus, ischemia, restenosis, vascular or perivascular diseases, dyslipoproteinemia, high levels of low density lipoprotein cholesterol, high levels of very low density lipoprotein cholesterol, low levels of high density lipoproteins, high levels of lipoprotein Lp(a) cholesterol, high levels of apolipoprotein B, atherosclerosis such as intermittent claudication caused by arterial insufficiency, accelerated atherosclerosis, graft atherosclerosis, familial hypercholesterolemia (FH), familial combined hyperlipidemia (FCH), lipoprotein lipase deficiencies such as hypertriglyceridemia, hypoalphalipoproteinemia, and hypercholesterolemia lipoprotein). In some embodiments, the dyslipidemic disorders is associated with Familial Primary Hypoalphalipoproteinemia (FPHA), such as Tangier's disease, ABCA1 deficiency, ApoA-I deficiency, LCAT deficiency or Fish-eye disease.

“IUSDEC” means an “inverted U-shaped dose-effect curve”. IUSDEC is a nonlinear relationship between the dose of a therapeutic agent and the patient response. The effects of increasing dosages of a given therapeutic appear to increase up to a maximum (the portion of the dose response curve with a positive slope), after which (the inflection point) the effects decrease (the portion of the dose response curve with a negative slope).

“HDL Therapeutic” means a therapeutic agent useful for treating hypercholesterolemia or hyperlipidemia and related disease conditions. Examples of HDL Therapeutics include HDL mimetic lipoprotein complexes (e.g., CER-001, CSL-111, CSL-112, CER-522, ETC-216) and small molecules (e.g., statins).

“HDL Marker” means a molecular marker whose expression correlates with the IUSDEC in response to treatment with HDL mimetics. Exemplary HDL Markers are ABCA1, ABCG1, ABCG5, ABCG8 and SREBP1. HDL Markers can be assayed at the mRNA or protein levels, for example as described in Section 6.2.

### 6.2. Companion Diagnostic Methods

Reverse cholesterol transport (RCT) is a pathway by which accumulated cholesterol is transported from the vessel wall to the liver for excretion, thus preventing atherosclerosis. Major constituents of RCT include acceptors such as high-density lipoprotein (HDL) and apolipoprotein A-I (ApoA-I), and enzymes such as lecithin:cholesterol acyltransferase (LCAT), phospholipid transfer protein (PLTP), hepatic lipase (HL) and cholesterol ester transfer protein (CETP). A critical part of RCT is cholesterol efflux, in which accumulated cholesterol is removed from macrophages, e.g., in the subintima of the vessel wall, by ATP-binding membrane cassette transporters A1 (ABCA1) and G1 (ABCG1) or by other mechanisms, including passive diffusion, scavenger receptor B1 (SR-B1), caveolins and sterol 27-hydroxylase, and collected by HDL and ApoA-I. Esterified cholesterol in the HDL is then delivered to the liver for excretion. The sterol regulatory element binding factor 1 gene (SREBP1) impacts RCT by regulating the biosynthesis of fatty acids and cholesterol.

The present disclosure is based in part on the discovery of IUSDEC-type response to treatment with HDL Therapeutics. The present disclosure is further based in part on the discovery of mechanisms of action underlying the HDL Therapeutic IUSDEC, namely the downregulation of expression of proteins (referred to herein as HDL Markers) involved in cholesterol efflux (e.g., ABCA1, ABCG1) or regulation of the RCT pathway (e.g., SREBP1) in response to treatment with HDL Therapeutics. It has been discovered that the downregulation of such proteins correlates with the IUSDEC in response to treatment with HDL Therapeutics.

The present disclosure relates in part to the use of this phenomenon to diagnose, prognose and dose optimize HDL Therapeutics in order to take advantage of the dose in the dose-response curve near the inflection point, i.e., in which the dose-response relationship is maximized.

The present disclosure relates in part to the use of this phenomenon to diagnose, prognose and dose optimize an HDL Therapeutic in order to take advantage of the dose in the dose-response curve near the inflection point, i.e., in which the dose-response relationship is optimized while not using an excess of HDL Therapeutic.

In other aspects, the present disclosure relates to the identification of therapeutic doses and dosing schedules that minimize impact on expression and/or function of HDL Markers in mediating cholesterol efflux, e.g., from a monocyte, macrophage or mononuclear cell. In some aspects, doses are selected that do not reduce expression of one or more HDL Markers by more than a defined cutoff point, e.g., 10%, 20%, 30%, 40%, 50%, 60%, 70% or 80% a reference amount of the HDL Marker. In certain embodiments, the cutoff is selected from any range of the reference bounded by any two of the foregoing cutoff amounts, e.g., 20%-80%, 30%-70%, 40%-60%, 10%-50%, 10%-40%, 20%-50%, and so on and so forth, may range from 20% to 80% of. The reference can be the subject's own baseline or some population average. The population can be an age-, gender- and/or disease risk factor (e.g., genetic or lifestyle risk factor) matched population. The population average can be a normal population or a population suffering from the same or similar condition as the subject. The particular HDL Marker and cutoff point will depend on the particular HDL Therapeutic, the subject's condition, and other therapies the subject may be receiving.

In some aspects, particularly where combination therapy is involved, a dose is selected that does not reduce the expression of one or more HDL Markers by more than 20%, in some embodiments no more than 10% and in yet other embodiments that does not the expression of one or more HDL Markers at all.

The use of HDL Markers as described herein can be used to optimize any of the treatment methods of Section 6.6. In certain embodiments, the disclosure provides a method of identifying a dose of an HDL Therapeutic effective to mobilize cholesterol in a subject. In some embodiments, the method comprises: (a) administering a first dose of an HDL Therapeutic to a subject, (b) following administering said first dose, measuring expression levels of one or more HDL Markers in a test sample from the subject, preferably said subject's circulating monocytes, macrophages or mononuclear cells, to evaluate the effect of said first dose on said expression levels; and (c) (i) if the subject's expression levels of one or more HDL Markers are reduced by more than a cutoff amount, administering a second dose of said HDL Therapeutic, wherein the second dose of said HDL Therapeutic is lower than the first dose; or (ii) if the subject's expression levels of one or more HDL Markers are not reduced by more than the cutoff amount, treating the subject with the first dose of said HDL Therapeutic.

In certain embodiments, the disclosure provides a method for monitoring the efficacy of an HDL Therapeutic in a subject. In some embodiments, the method comprises: (a) treating a subject with an HDL Therapeutic according to a first dosing schedule, (b) measuring expression levels of one or more HDL Markers in a test sample from the subject, preferably said subject's circulating monocytes, macrophages or mononuclear cells, to evaluate the effect of said first dosing schedule on said expression levels; and (c) (i) if the subject's expression levels of one or more HDL Markers are reduced by more than an upper cutoff amount, treating the subject with the HDL Therapeutic according to a second dosing schedule, wherein the second dosing schedule comprises one or more of: administering a lower dose of the HDL Therapeutic, infusing the HDL Therapeutic into the subject over a longer period of time, and administering the HDL Therapeutic to the subject on a less frequent basis; (ii) if the subject's expression levels of one or more HDL Markers are not reduced by more than a lower cutoff amount, treating the subject with the HDL Therapeutic according to a second dosing schedule, wherein the second dosing schedule comprises one or more of: administering a higher dose of the HDL Therapeutic, infusing the HDL Therapeutic into the subject over a shorter period of time, and administering the HDL Therapeutic to the subject on a more frequent basis; or (iii) if the subject's expression levels of one or more HDL Markers are reduced by an amount between the upper and lower cutoff amounts, continuing to treat the subject according to the first dosing schedule.

The cutoff amount may be relative to the subject's own baseline prior to said administration or the cutoff amount may be relative to a control amount such as a population average from e.g., healthy subjects or a population with the same disease condition as the subject.

In certain embodiments, the disclosure provides a method of identifying a dose of an HDL Therapeutic effective to mobilize cholesterol. In some embodiments, the method comprises: (a) administering a first dose of an HDL Therapeutic to a population of subjects; (b) following administering said first dose, measuring expression levels of one or more HDL Markers in a test sample from the subjects, preferably said subjects' circulating monocytes, macrophages or mononuclear cells, to evaluate the effect of said first dose on said expression levels; (c) administering a second dose of said HDL Therapeutic, wherein the second dose of said HDL Therapeutic is greater or lower than the first dose; (d) following administering said second dose, measuring expression levels of one or more HDL Markers in a test sample from the subjects, preferably said subjects' circulating monocytes, macrophages or mononuclear cells, to evaluate the effect of said first dose on said expression levels; (e) optionally repeating steps (c) and (d) with one or more additional doses of said HDL Therapeutic; and (f) identifying the highest dose that does not reduce expression levels of one or more HDL Markers in by more than a cutoff amount, thereby identifying a dose of said HDL Therapeutic effective to mobilize cholesterol.

In certain embodiments, following administration of said second dose, expression levels of one or more HDL Markers in said test sample (e.g., circulating monocytes, macrophages or mononuclear cells) is measured to evaluate the effect of said second dose on said expression levels. If the subject's expression levels of one or more HDL Markers are reduced by more than a cutoff amount, a third dose of said HDL Therapeutic may be administered, wherein the third dose of said HDL Therapeutic is lower than the second dose.

In certain embodiments, the disclosure provides a method for treating a subject in need of an HDL Therapeutic. In some embodiments, the method comprises administering to subject a combination of: (a) a lipoprotein complex in a dose that does not reduce expression of one or more HDL Markers in a test sample from said subject (e.g., said subject's circulating monocytes, macrophages or mononuclear cells) by more than 20% or more than 10% as compared to the subject's baseline amount; and (b) a cholesterol reducing therapy, optionally selected from a bile-acid resin, niacin, a statin, a fibrate, a PCSK9 inhibitor, ezetimibe, and a CETP inhibitor.

In certain embodiments, the disclosure provides a method for treating a subject in need of an HDL Therapeutic. In some embodiments, the method comprises administering to subject a combination of: (a) a lipoprotein complex in a dose that does not reduce expression of one or more HDL Markers in a test sample from said subject (e.g., said subject's circulating monocytes, macrophages or mononuclear cells) by more than 20% or more than 10% as compared to a control amount; and (b) a cholesterol reducing therapy, optionally selected from a bile-acid resin, niacin, a statin, a fibrate, a PCSK9 inhibitor, ezetimibe, and a CETP inhibitor.

The control amount may be the population average, e.g., the population average from healthy subjects or a population with the same disease condition as the subject. The subject may be human or the population of subjects is a population of human subjects. The subject may be a non-human animal, e.g., mouse, or the population of subjects may be a population of non-human animals.

In certain embodiments of the methods described herein, at least one HDL Marker is ABCA1. For example, ABCA1 mRNA expression levels or ABCA1 protein expression levels are measured. The ABCA1 cutoff amount may be 10%, 20%, 30%, 40%, 50%, 60%, 70% or 80%, or selected from any range bounded by any two of the foregoing cutoff amounts, e.g., 20%-80%, 30%-70%, 40%-60%, 10%-50%, 10%-40%, 20%-50%, and so on and so forth. ABCA1 expression levels may be measured 2-12 hours, 4-10 hours, 2-8 hours, 2-6 hours, 4-6 hours or 4-8 hours after administration.

In certain embodiments of the methods described herein, at least one HDL Marker is ABCG1. For example, ABCG1 mRNA expression levels or ABCG1 protein expression levels are measured. The ABCG1 cutoff amount may be 10%, 20%, 30%, 40%, 50%, 60%, 70% or 80%, or selected from any range bounded by any two of the foregoing cutoff amounts, e.g., 20%-80%, 30%-70%, 40%-60%, 10%-50%, 10%-40%, 20%-50%, and so on and so forth. ABCG1 expression levels may be measured 2-12 hours, 4-10 hours, 2-8 hours, 2-6 hours, 4-6 hours or 4-8 hours after administration.

In certain embodiments of the methods described herein, at least one HDL Marker is SREBP-1. For example, SREBP-1 mRNA expression levels or SREBP-1 protein expression levels are measured. The SREBP-1 cutoff amount may be 10%, 20%, 30%, 40%, 50%, 60%, 70% or 80%, or selected from any range bounded by any two of the foregoing cutoff amounts, e.g., 20%-80%, 30%-70%, 40%-60%, 10%-50%, 10%-40%, 20%-50%, and so on and so forth. SREBP-1 expression levels may be measured 2-12 hours, 4-10 hours, 2-8 hours, 2-6 hours, 4-6 hours or 4-8 hours after administration.

In some embodiments, a dose is identified that does not alter or even increases the expression levels of one or more HDL Markers in the subject's circulating monocytes, macrophages or mononuclear cells. The levels can be increased by at least 10%, at least 20%, at least 30%, at least 40%, at least 50%, at least 60%, at least 70%, at least 80%, or in a range bounded by any two of the foregoing values, e.g., the levels can be increased by up to 10%, up to 20%, up to 50%, 10%-50%, 20%-60%, and so on and so forth.

In certain embodiments, the HDL Therapeutic is a lipoprotein complex. The lipoprotein complex may comprise an apolipoprotein such as ApoA-I, ApoA-II, ApoA-IV, ApoE or a combination thereof. The lipoprotein complex may comprise an apolipoprotein peptide mimic such as an ApoA-I, ApoA-II, ApoA-IV, or ApoE peptide mimic or a combination thereof. The lipoprotein complex may be CER-001, CSL-111, CSL-112, CER-522, or ETC-216. In other embodiments, the HDL Therapeutic is a delipidated or lipid poor lipoprotein.

In certain embodiments, the HDL Therapeutic is a small molecule such as a CETP inhibitor or a pantothenic acid derivative.

In certain embodiments, the methods described herein further comprise determining a cutoff amount. For example, the cutoff amount may be determined by generating a dose response curve for the HDL Therapeutic. The cutoff amount may be 25%, 40%, 50%, 60% or 75% of the expression level of the HDL Marker at the inflection point in the dose response curve. In particular embodiments, the cutoff is selected from a range bounded by any two of the foregoing cutoff values, e.g., 30%-70%, 40%-60%, 25%-50%, 25%-75% of the expression level of the HDL Marker at the inflection point in the dose response curve, and so on and so forth.

In certain embodiments, the disclosure provides a method of identifying a dose of an HDL Therapeutic suitable for therapy. In some embodiments, the method comprises: (a) administering one or more doses of an HDL Therapeutic to a subject; (b) measuring expression levels of one or more HDL Markers in said subject's circulating monocytes, macrophages or mononuclear cells following each dose; and (c) identifying the maximum dose that does not raise expression levels of said one or more HDL Markers by more than 0%, more than 10% or more than 20%, thereby identifying a dose of an HDL Therapeutic suitable for therapy.

In certain embodiments, the disclosure provides a method of identifying a dose of an HDL Therapeutic suitable for therapy. In some embodiments, the method comprises: (a) administering one or more doses of an HDL Therapeutic to a population of subjects; (b) measuring expression levels of one or more HDL Markers in said subjects' circulating monocytes, macrophages or mononuclear cells following each dose; and (c) identifying the maximum dose that does not raise expression levels of said one or more HDL Markers by more than 0%, more than 10% or more than 20% in said subjects, thereby identifying a dose of an HDL Therapeutic suitable for therapy.

In certain embodiments, the disclosure provides a method of identifying a dose of an HDL Therapeutic suitable for therapy. In some embodiments, the method comprises identifying the highest dose of the HDL therapeutic that does not reduce cellular cholesterol efflux by more than 0%, more than 10% or more than 20%. A method of identifying a dose of an HDL Therapeutic suitable for therapy may comprise: (a) administering one or more doses of an HDL Therapeutic to a subject or population of subjects; (b) measuring cholesterol efflux in cells from said subject or population of subjects; and (c) identifying the maximum dose that does not reduce cholesterol efflux by more than 0%, more than 10% or more than 20% in said subjects, thereby identifying a dose of an HDL Therapeutic suitable for therapy.

In certain embodiments, the disclosure provides a method of identifying a dosing interval of an HDL Therapeutic suitable for therapy. In some embodiments, the method comprises identifying the highest dose of the most frequent dosing regimen of the HDL therapeutic that does not reduce cellular cholesterol efflux by more than 0%, more than 10% or more than 20%. A method of identifying a dosing interval of an HDL Therapeutic suitable for therapy may comprise: (a) administering an HDL Therapeutic to a subject or population of subjects according to one or more dosing frequencies; (b) measuring cholesterol efflux in cells from said subject or population of subjects; and (c) identifying the maximum dosing frequency that does not reduce cholesterol efflux by more than 50% to 100% in said subjects, thereby identifying a dose of an HDL Therapeutic suitable for therapy.

### 6.3. HDL Therapeutics

HDL Therapeutics of the disclosure include lipoprotein complexes, delipidated or lipid poor lipoproteins, peptides, fusion proteins and HDL mimetics. It is noted that “lipoproteins” and “apolipoproteins” are used interchangeably herein.

Lipoprotein complexes may comprise a protein fraction (e.g., an apolipoprotein fraction) and a lipid fraction (e.g., a phospholipid fraction). The protein fraction includes one or more lipid-binding proteins, such as apolipoproteins, peptides, or apolipoprotein peptide analogs or mimetics capable of mobilizing cholesterol when present in a lipoprotein complex. Non-limiting examples of such apolipoproteins and apolipoprotein peptides include ApoA-I, ApoA-II, ApoA-IV, ApoA-V and ApoE; preferably in mature form. Lipid-binding proteins also active polymorphic forms, isoforms, variants and mutants as well as truncated forms of the foregoing apolipoproteins, the most common of which are Apolipoprotein A-I_{Milano} (ApoA-I_{M}), Apolipoprotein A-I_{Paris} (ApoA-I_{P}), and Apolipoprotein A-I_{Zaragoza} (ApoA-I_{Z}). Apolipoproteins mutants containing cysteine residues are also known, and can also be used (see, e.g., U.S. Publication No. 2003/0181372). The apolipoproteins may be in the form of monomers or dimers, which may be homodimers or heterodimers. For example, homo- and heterodimers (where feasible) of ApoA-I (Duverger et al., 1996, Arterioscler. Thromb. Vasc. Biol. 16(12):1424-29), ApoA-I_{M} (Franceschini et al., 1985, J. Biol. Chem. 260:1632-35), ApoA-I_{P} (Daum et al., 1999, J. Mol. Med. 77:614-22), ApoA-II (Shelness et al., 1985, J. Biol. Chem. 260(14):8637-46; Shelness et al., 1984, J. Biol. Chem. 259(15):9929-35), ApoA-IV (Duverger et al., 1991, Euro. J. Biochem. 201(2):373-83), ApoE (McLean et al., 1983, J. Biol. Chem. 258(14):8993-9000), ApoJ and ApoH may be used.

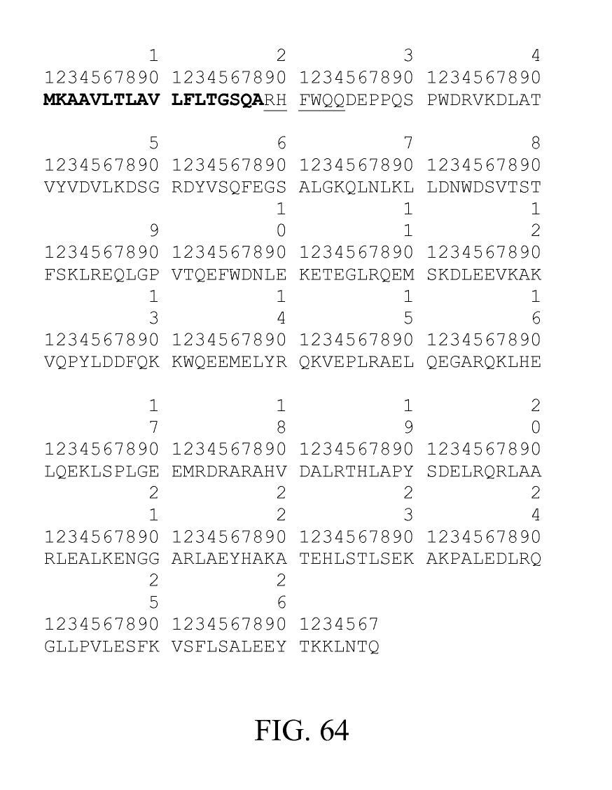

The apolipoproteins may include residues corresponding to elements that facilitate their isolation, such as His tags, or other elements designed for other purposes, so long as the apolipoprotein retains some biological activity when included in a complex. In a specific embodiment, the apolipoprotein fraction consists essentially of ApoA-I, most preferably of a single isoform. ApoA-I in lipoprotein complexes can have at least 90% or at least 95% sequence identity to a protein corresponding to amino acids 25 to 267 of the ApoA-I lipoprotein of FIG. 64 (SEQ ID NO:1). Optionally, ApoA-I further comprises an aspartic acid at the position corresponding to the full length ApoA-I amino acid 25 of SEQ ID NO:1 (and position 1 of the mature protein). Preferably, at least 75%, at least 80%, at least 85%, at least 90% or at least 95% of the ApoA-I is correctly processed, mature protein (i.e., lacking the signal and propeptide sequences) and not oxidized, deamidated and/or truncated.

Peptides and peptide analogs that correspond to apolipoproteins, as well as agonists that mimic the activity of ApoA-I, ApoA-I, ApoA-II, ApoA-IV, and ApoE, can be used. Non-limiting examples of peptides and peptide analogs are disclosed in U.S. Pat. Nos. 6,004,925, 6,037,323 and 6,046,166 (issued to Dasseux et al.), U.S. Pat. No. 5,840,688 (issued to Tso), U.S. Publication Nos. 2004/0266671, 2004/0254120, 2003/0171277 and 2003/0045460 (to Fogelman), U.S. Publication No. 2003/0087819 (to Bielicki) and PCT Publication No. WO2010/093918 (to Dasseux et al.), the disclosures of which are incorporated herein by reference in their entireties. These peptides and peptide analogues can be composed of L-amino acid or D-amino acids or mixture of L- and D-amino acids. They may also include one or more non-peptide or amide linkages, such as one or more well-known peptide/amide isosteres. Such “peptide and/or peptide mimetic” apolipoproteins can be synthesized or manufactured using any technique for peptide synthesis known in the art, including, e.g., the techniques described in U.S. Pat. Nos. 6,004,925, 6,037,323 and 6,046,166.

The lipoproteins can be used as HDL Therapeutics in delipidated forms, or in a lipoprotein complex containing a lipid fraction in addition to a protein fraction. The lipid fraction typically includes one or more phospholipids which can be neutral, negatively charged, positively charged, or a combination thereof. The fatty acid chains on phospholipids are preferably from 12 to 26 or 16 to 26 carbons in length and can vary in degree of saturation from saturated to mono-unsaturated. Exemplary phospholipids include small alkyl chain phospholipids, egg phosphatidylcholine, soybean phosphatidylcholine, dipalmitoylphosphatidylcholine, dimyristoylphosphatidylcholine, distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine dioleophosphatidylethanolamine, dilauroylphosphatidylglycerol phosphatidylcholine, phosphatidylserine, phosphatidylethanolamine, phosphatidylinositol, phosphatidylglycerols, diphosphatidylglycerols such as dimyristoylphosphatidylglycerol, dipalmitoylphosphatidylglycerol, distearoylphosphatidylglycerol, dioleoylphosphatidylglycerol, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, brain sphingomyelin, egg sphingomyelin, milk sphingomyelin, palmitoyl sphingomyelin, phytosphingomyelin, dipalmitoylsphingomyelin, distearoylsphingomyelin, dipalmitoylphosphatidylglycerol salt, phosphatidic acid, galactocerebroside, gangliosides, cerebrosides, dilaurylphosphatidylcholine, (1,3)-D-mannosyl-(1,3)diglyceride, aminophenylglycoside, 3-cholesteryl-6′-(glycosylthio)hexyl ether glycolipids, and cholesterol and its derivatives. Phospholipid fractions including SM and palmitoylsphingomyelin can optionally include small quantities of any type of lipid, including but not limited to lysophospholipids, sphingomyelins other than palmitoylsphingomyelin, galactocerebroside, gangliosides, cerebrosides, glycerides, triglycerides, and cholesterol and its derivatives.

In certain embodiments, the lipid fraction contains at least one neutral phospholipid and, optionally, one or more negatively charged phospholipids. In lipoprotein complexes that include both neutral and negatively charged phospholipids, the neutral and negatively charged phospholipids can have fatty acid chains with the same or different number of carbons and the same or different degree of saturation. In some instances, the neutral and negatively charged phospholipids will have the same acyl tail, for example a C16:0, or palmitoyl, acyl chain. In specific embodiments, particularly those in which egg SM is used as the neutral lipid, the weight ratio of the apolipoprotein fraction:lipid fraction ranges from about 1:2.7 to about 1:3 (e.g., 1:2.7).

Any phospholipid that bears at least a partial negative charge at physiological pH can be used as the negatively charged phospholipid. Non-limiting examples include negatively charged forms, e.g., salts, of phosphatidylinositol, a phosphatidylserine, a phosphatidylglycerol and a phosphatidic acid. In a specific embodiment, the negatively charged phospholipid is 1,2-dipalmitoyl-sn-glycero-3-[phospho-rac-(1-glycerol)], or DPPG, a phosphatidylglycerol. Preferred salts include potassium and sodium salts.

In some embodiments, an HDL Therapeutic is a lipoprotein complex described in U.S. Pat. No. 8,206,750 or WO 2012/109162 (and its U.S. counterpart, US 2012/0232005), the contents of each of which are incorporated herein in its entirety by reference. In particular embodiments, the protein component of the lipoprotein complex is as described in Section 6.1 and preferably in Section 6.1.1 of WO 2012/109162 (and US 2012/0232005), the lipid component is as described in Section 6.2 of WO 2012/109162 (and US 2012/0232005), which can optionally be complexed together in the amounts described in Section 6.3 of WO 2012/109162 (and US 2012/0232005). The contents of each of these sections are incorporated by reference herein. In certain aspects, the lipoprotein complex is in a population of complexes that is at least 85%, at least 90%, at least 95%, at least 97%, or at least 99% homogeneous, as described in Section 6.4 of WO 2012/109162 (and US 2012/0232005), the contents of which are incorporated by reference herein.

In a specific embodiment, the lipoprotein complex consists essentially of 2-4 ApoA-I equivalents, 2 molecules of charged phospholipid, 50-80 molecules of lecithin and 20-50 molecules of SM.

In another specific embodiment, the lipoprotein complex consists essentially of 2-4 ApoA-I equivalents, 2 molecules of charged phospholipid, 50 molecules of lecithin and 50 molecules of SM.

In yet another specific embodiment, the lipoprotein complex consists essentially of 2-4 ApoA-I equivalents, 2 molecules of charged phospholipid, 80 molecules of lecithin and 20 molecules of SM.

In yet another specific embodiment, the lipoprotein complex consists essentially of 2-4 ApoA-I equivalents, 2 molecules of charged phospholipid, 70 molecules of lecithin and 30 molecules of SM.

In yet another specific embodiment, the lipoprotein complex consists essentially of 2-4 ApoA-I equivalents, 2 molecules of charged phospholipid, 60 molecules of lecithin and 40 molecules of SM.

In a specific embodiment, lipoprotein complex is a ternary complex in which the lipid component consists essentially of about 90 to 99.8 wt % SM and about 0.2 to 10 wt % negatively charged phospholipid, for example, about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt %, or 0.2-10 wt % total negatively charged phospholipid(s). In another specific embodiment, the lipoprotein complex is a ternary complex in which the lipid fraction consists essentially of about 90 to 99.8 wt % lecithin and about 0.2 to 10 wt % negatively charged phospholipid, for example, about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt % or 0.2-10 wt % total negatively charged phospholipid(s).

In still another specific embodiment, the lipoprotein complex is a quaternary complex in which the lipid fraction consists essentially of about 9.8 to 90 wt % SM, about 9.8 to 90 wt % lecithin and about 0.2-10 wt % negatively charged phospholipid, for example, from about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt %, to 0.2-10 wt % total negatively charged phospholipid(s).

In another specific embodiment, the lipoprotein complex consists of 33 wt % proApoAI, 65 wt % sphingomyelin and 2 wt % phosphatidylglycerol.

In another specific embodiment, the lipoprotein complex comprises an ApoA-I apolipoprotein and a lipid fraction, wherein the lipid fraction consists essentially of sphingomyelin and about 3 wt % of a negatively charged phospholipid, wherein the molar ratio of the lipid fraction to the ApoA-I apolipoprotein is about 2:1 to 200:1, and wherein said lipoprotein complex is a small or large discoidal particle containing 2-4 ApoA-I equivalents.

The complexes can include a single type of lipid-binding protein, or mixtures of two or more different lipid-binding proteins, which may be derived from the same or different species. Although not required, the lipoprotein complexes will preferably comprise lipid-binding proteins that are derived from, or correspond in amino acid sequence to, the animal species being treated, in order to avoid inducing an immune response to the therapy. Thus, for treatment of human patients, lipid-binding proteins of human origin are preferably used in the complexes of the disclosure. The use of peptide mimetic apolipoproteins may also reduce or avoid an immune response.

In preferred embodiments, the lipid component includes two types of phospholipids: a sphingomyelin (SM) and a negatively charged phospholipid. SM is a “neutral” phospholipid in that it has a net charge of about zero at physiological pH. Thus, as used herein, the expression “SM” includes sphingomyelins derived or obtained from natural sources, as well as analogs and derivatives of naturally occurring SMs that are impervious to hydrolysis by LCAT, as is naturally occurring SM.

The SM may be obtained from virtually any source. For example, the SM may be obtained from milk, egg or brain. SM analogues or derivatives may also be used. Non-limiting examples of useful SM analogues and derivatives include, but are not limited to, palmitoylsphingomyelin, N-palmitoyl-4-hydroxysphinganine-1-phosphocholine (a form of phytosphingomyelin), palmitoylsphingomyelin, stearoylsphingomyelin, D-erythro-N-16:0-sphingomyelin and its dihydro isomer, D-erythro-N-16:0-dihydro-sphingomyelin. Synthetic SM such as synthetic palmitoylsphingomyelin or N-palmitoyl-4-hydroxysphinganine-1-phosphocholine (phytosphingomyelin) can be used in order to produce more homogeneous complexes and with fewer contaminants and/or oxidation products than sphingolipids of animal origin.

Sphingomyelins isolated from natural sources may be artificially enriched in one particular saturated or unsaturated acyl chain. For example, milk sphingomyelin (Avanti Phospholipid, Alabaster, Ala.) is characterized by long saturated acyl chains (i.e., acyl chains having 20 or more carbon atoms). In contrast, egg sphingomyelin is characterized by short saturated acyl chains (i.e., acyl chains having fewer than 20 carbon atoms). For example, whereas only about 20% of milk sphingomyelin comprises C16:0 (16 carbon, saturated) acyl chains, about 80% of egg sphingomyelin comprises C16:0 acyl chains. Using solvent extraction, the composition of milk sphingomyelin can be enriched to have an acyl chain composition comparable to that of egg sphingomyelin, or vice versa.

The SM may be semi-synthetic such that it has particular acyl chains. For example, milk sphingomyelin can be first purified from milk, then one particular acyl chain, e.g., the C16:0 acyl chain, can be cleaved and replaced by another acyl chain. The SM can also be entirely synthesized, by e.g., large-scale synthesis. See, e.g., Dong et al., U.S. Pat. No. 5,220,043, entitled Synthesis of D-erythro-sphingomyelins, issued Jun. 15, 1993; Weis, 1999, Chem. Phys. Lipids 102 (1-2):3-12.

The lengths and saturation levels of the acyl chains comprising a semi-synthetic or a synthetic SM can be selectively varied. The acyl chains can be saturated or unsaturated, and can contain from about 6 to about 24 carbon atoms. Each chain may contain the same number of carbon atoms or, alternatively each chain may contain different numbers of carbon atoms. In some embodiments, the semi-synthetic or synthetic SM comprises mixed acyl chains such that one chain is saturated and one chain is unsaturated. In such mixed acyl chain SMs, the chain lengths can be the same or different. In other embodiments, the acyl chains of the semi-synthetic or synthetic SM are either both saturated or both unsaturated. Again, the chains may contain the same or different numbers of carbon atoms. In some embodiments, both acyl chains comprising the semi-synthetic or synthetic SM are identical. In a specific embodiment, the chains correspond to the acyl chains of a naturally-occurring fatty acid, such as for example myristic, oleic, palmitic, stearic, linoleic, linonenic, or arachidonic acid. In another embodiment, SM with saturated or unsaturated functionalized chains is used. In another specific embodiment, both acyl chains are saturated and contain from 6 to 24 carbon atoms.

In preferred embodiments, the SM is palmitoyl SM, such as synthetic palmitoyl SM, which has C16:0 acyl chains, or is egg SM, which includes as a principal component palmitoyl SM.

In a specific embodiment, functionalized SM, such as phytosphingomyelin, is used.

The lipid component preferably includes a negatively charged phospholipid, i.e., phospholipids that have a net negative charge at physiological pH. The negatively charged phospholipid may comprise a single type of negatively charged phospholipid, or a mixture of two or more different, negatively charged, phospholipids. In some embodiments, the charged phospholipids are negatively charged glycerophospholipids. Specific examples of suitable negatively charged phospholipids include, but are not limited to, a 1,2-dipalmitoyl-sn-glycero-3-[phospho-rac-(1-glycerol)], a phosphatidylglycerol, a phospatidylinositol, a phosphatidylserine, and a phosphatidic acid. In some embodiments, the negatively charged phospholipid comprises one or more of phosphatidylinositol, phosphatidylserine, phosphatidylglycerol and/or phosphatidic acid. In a specific embodiment, the negatively charged phospholipid consists of 1,2-dipalmitoyl-sn-glycero-3-[phospho-rac-(1-glycerol)], or DPPG.

Like the SM, the negatively charged phospholipids can be obtained from natural sources or prepared by chemical synthesis. In embodiments employing synthetic negatively charged phospholipids, the identities of the acyl chains can be selectively varied, as discussed above in connection with SM. In some embodiments of the negatively charged lipoprotein complexes described herein, both acyl chains on the negatively charged phospholipids are identical. In some embodiments, the acyl chains on the SM and the negatively charged phospholipids are all identical. In a specific embodiment, the negatively charged phospholipid(s), and/or SM all have C16:0 or C16:1 acyl chains. In a specific embodiment the fatty acid moiety of the SM is predominantly C16:1 palmitoyl. In one specific embodiment, the acyl chains of the charged phospholipid(s) and/or SM correspond to the acyl chain of palmitic acid.

The phospholipids used are preferably at least 95% pure, and/or have reduced levels of oxidative agents. Lipids obtained from natural sources preferably have fewer polyunsaturated fatty acid moieties and/or fatty acid moieties that are not susceptible to oxidation. The level of oxidation in a sample can be determined using an iodometric method, which provides a peroxide value, expressed in milli-equivalent number of isolated iodines per kg of sample, abbreviated meq O/kg. See, e.g., Gray, J. I., Measurement of Lipid Oxidation: A Review, Journal of the American Oil Chemists Society, Vol. 55, p. 539-545 (1978); Heaton, F. W. and Un N., Improved Iodometric Methods for the Determination of Lipid Peroxides, Journal of the Science of food and Agriculture, vol 9. P, 781-786 (1958). Preferably, the level of oxidation, or peroxide level, is low, e.g., less than 5 meq O/kg, less than 4 meq O/kg, less than 3 meq O/kg, or less than 2 meq O/kg.

Lipid components including SM and palmitoylsphingomyelin can optionally include small quantities of additional lipids. Virtually any type of lipids may be used, including, but not limited to, lysophospholipids, galactocerebroside, gangliosides, cerebrosides, glycerides, triglycerides, and cholesterol and its derivatives.

When included, such optional lipids will typically comprise less than about 15 wt % of the lipid fraction, although in some instances more optional lipids could be included. In some embodiments, the optional lipids comprise less than about 10 wt %, less than about 5 wt %, or less than about 2 wt %. In some embodiments, the lipid fraction does not include optional lipids.

In a specific embodiment, the phospholipid fraction contains egg SM or palmitoyl SM or phytosphingomyelin and DPPG in a weight ratio (SM: negatively charged phospholipid) ranging from 90:10 to 99:1, more preferably ranging from 95:5 to 98:2. In one embodiment, the weight ratio is 97:3.

The lipoprotein complexes can also be used as carriers to deliver hydrophobic, lipophilic or apolar active agents for a variety of therapeutic or diagnostic applications. For such applications, the lipid component can further include one or more hydrophobic, lipophilic or apolar active agents, including but not limited to fatty acids, drugs, nucleic acids, vitamins, and/or nutrients. Suitable hydrophobic, lipophilic or apolar active agents are not limited by therapeutic category, and can be, for example, analgesics, anti-inflammatory agents, antihelmimthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malariale, anti-migrainc agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, leukotriene inhibitors, macrolides, muscle relaxants, nutritional agents, nucleic acids (e.g., small interfering RNAs), opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof.

The molar ratio of the lipid component to the protein component of the lipoprotein complexes can vary, and will depend upon, among other factors, the identity(ies) of the apolipoprotein comprising the protein component, the identities and quantities of the lipids comprising the lipid component, and the desired size of the lipoprotein complex. Because the biological activity of apolipoproteins such as ApoA-I are thought to be mediated by the amphipathic helices comprising the apolipoprotein, it is convenient to express the apolipoprotein fraction of the lipid:apolipoprotein molar ratio using ApoA-I protein equivalents. It is generally accepted that ApoA-I contains 6-10 amphipathic helices, depending upon the method used to calculate the helices. Other apolipoproteins can be expressed in terms of ApoA-I equivalents based upon the number of amphipathic helices they contain. For example, ApoA-I_{M}, which typically exists as a disulfide-bridged dimer, can be expressed as 2 ApoA-I equivalents, because each molecule of ApoA-I_{M} contains twice as many amphipathic helices as a molecule of ApoA-I. Conversely, a peptide apolipoprotein that contains a single amphipathic helix can be expressed as a 1/10-1/6 ApoA-I equivalent, because each molecule contains 1/10-1/6 as many amphipathic helices as a molecule of ApoA-I. In general, the lipid:ApoA-I equivalent molar ratio of the lipoprotein complexes (defined herein as “Ri”) will range from about 105:1 to 110:1. In some embodiments, the Ri is about 108:1. Ratios in weight can be obtained using a MW of approximately 650-800 for phospholipids.

In some embodiments, the molar ratio of lipid:ApoA-I equivalents (“RSM”) ranges from about 80:1 to about 110:1, e.g., about 80:1 to about 100:1. In a specific example, the RSM for lipoprotein complexes can be about 82:1.

In preferred embodiments, the lipoprotein complexes are negatively charged lipoprotein complexes which comprise a protein fraction which is preferably mature, full-length ApoA-I, and a lipid fraction comprising a neutral phospholipid, sphingomyelin (SM), and negatively charged phospholipid.

In a specific embodiment, the lipid component contains egg SM or palmitoyl SM or phytoSM and DPPG in a weight ratio (SM: negatively charged phospholipid) ranging from 90:10 to 99:1, more preferably ranging from 95:5 to 98:2, e.g., 97:3.

In specific embodiments, the ratio of the protein component to lipid component typically ranges from about 1:2.7 to about 1:3, with 1:2.7 being preferred. This corresponds to molar ratios of ApoA-I protein to lipid ranging from approximately 1:90 to 1:140. In some embodiments, the molar ratio of protein to lipid in the lipoprotein complex is about 1:90 to about 1:120, about 1:100 to about 1:140, or about 1:95 to about 1:125.

In particular embodiments, the complex is CER-001, CSL-111, CSL-112, CER-522 or ETC-216.

CER-001 comprises ApoA-I, sphingomyelin (SM) and DPPG in a 1:2.7 lipoprotein wt:total phospholipid wt ratio with a SM:DPPG wt:wt ratio of 97:3. Preferably, the SM is egg SM, although synthetic SM or phyto SM can be substituted. In some embodiments, the complex is made according to the method described in Example 4 of WO 2012/109162.

CSL-111 is a reconstituted human ApoA-I purified from plasma complexed with soybean phosphatidylcholine (SBPC) (Tardif et al., 2007, JAMA 297:1675-1682).

CSL-112 is a formulation of ApoA-I purified from plasma and reconstituted to form HDL suitable for intravenous infusion (Diditchenko et al., 2013, DOI 10.1161/ATVBAHA.113.301981).

ETC-216 (also known as MDCO-216) is a lipid-depleted form of HDL containing recombinant ApoA-I_{Milano}. See Nicholls et al., 2011, Expert Opin Biol Ther. 11(3):387-94. doi: 10.1517/14712598.2011.557061.

In another embodiment, the complex is CER-522, a lipoprotein complex comprising a combination of three phospholipids and a 22 amino acid peptide, CT80522:

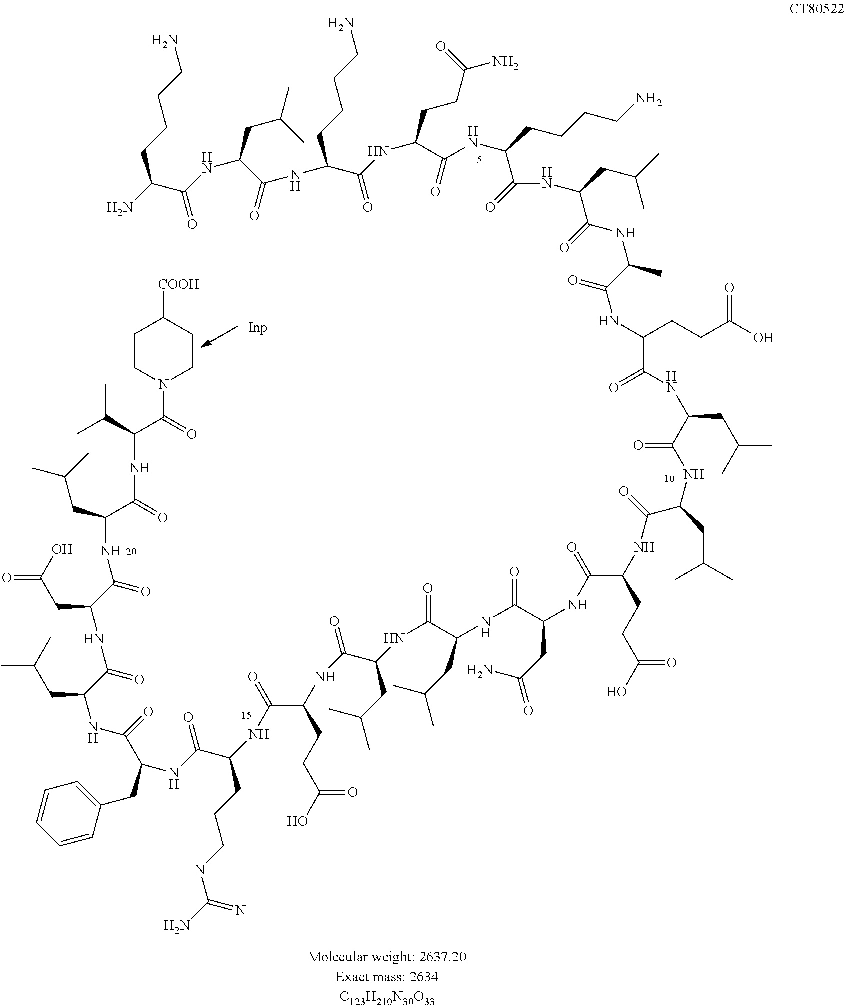 [SMILES: CC(C)C[C@H](NC(=O)C(CCC(=O)O)NC(=O)[C@H](C)NC(=O)[C@H](CC(C)C)NC(=O)[C@H](CCCCN)NC(=O)[C@H](CCC(N)=O)NC(=O)[C@H](CCCCN)NC(=O)[C@H](CC(C)C)NC(=O)[C@@H](N)CCCCN)C(=O)N[C@@H](CC(C)C)C(=O)N[C@@H](CCC(=O)O)C(=O)N[C@@H](CC(N)=O)C(=O)N[C@@H](CC(C)C)C(=O)N[C@@H](CC(C)C)C(=O)N[C@@H](CCC(=O)O)C(=O)N[C@@H](CCCNC(=N)N)C(=O)N[C@@H](Cc1ccccc1)C(=O)N[C@@H](CC(C)C)C(=O)N[C@@H](CC(=O)O)C(=O)N[C@@H](CC(C)C)C(=O)N[C@H](C(=O)N1CCC(C(=O)O)CC1)C(C)C]

The phospholipid component of CER-522 consists of egg sphingomyelin, 1,2-dipalmitoyl-sn-glycero-3-phosphocholine (Dipalmitoylphosphatidylcholine, DPPC) and 1,2-dipalmitoyl-sn-glycero-3-[phospho-rac-(1-glycerol)] (Dipalmitoylphosphatidyl-glycerol, DPPG) in a 48.5:48.5:3 weight ratio. The ratio of peptide to total phospholipids in the CER-522 complex is 1:2.5 (w/w).

Additional examples of HDL Therapeutics include, but are not limited to, the lipoprotein complexes, delipidated apolipoproteins, peptides, fusion proteins and HDL mimetics described in U.S. Pat. Nos. 8,617,615; 8,206,750; 8,378,068; 7,994,120; 7,566,695; 7,312,190; 7,307,058; 7,273,848; 7,250,407; 7,211,565; 7,189,689; 7,189,411; 7,157,425; 6,900,177; 6,844,327; 6,753,313; 6,734,169; 6,716,816; 6,630,450; 6,602,854; 6,573,239; 6,455,088; 6,376,464; 6,329,341; 6,287,590; 6,265,377; 6,046,166; 6,037,323; 6,004,925; 6,743,778; 8,383,592; 8,101,565; 8,044,021; 7,985,728; 7,985,727; 8,568,766; 8,557,767; 8,404,635; 8,148,328; 8,048,851; 7,994,132; 7,820,784; 7,807,640; 7,723,303; 7,638,494; 7,531,514; 7,199,102; 7,166,578; 7,148,197; 7,144,862; 6,933,279; 6,930,085; 8,541,236; 8,148,323; 8,071,746; 7,572,771; 7,223,726; 8,163,699; 8,415,293; 7,691,965; 7,601,802; 7,439,323; 7,217,785; 8,158,601; 8,653,245; 8,557,962; 7,491,693; 7,749,315; 5,059,528; RE38,556; 6,258,596; 5,866,551; 6,953,840; 8,119,590; 7,193,056, 6,767,994; 6,617,134; 6,559,284; 6,454,950; 6,306,433; 6,107,467; 5,990,081; 5,876,968; 5,721,114; 8,343,932; 7,786,352; 8,536,117; 8,143,224; 7,781,219; 7,776,563; 7,390,504; 7,378,396; 6,897,039; 7,273,849; 8,637,460; 8,268,787; 8,048,851; 8,048,015; 8,030,281; 7,402,246; 7,393,826; 7,375,191; 7,361,739; 7,364,658; 7,361,739; 7,364,658; 7,361,739; 7,297,262; 7,297,261; 7,195,710; 7,166,223; 7,033,500; 6,897,039; 8,252,739; 7,847,079; 7,592,010; 7,550,432; 7,521,424; 7,507,414; 7,507,413; 7,482,013; 7,238,667; 7,094,577; 7,081,354; 7,056,701; 7,045,318; 7,041,478; 6,994,857; 6,989,365; 6,987,006; 6,972,322; 6,946,134; 6,926,898; 6,909,014; 6,905,688 and U.S. Patent Publication No. 20040266662 all of which are incorporated by reference in their entirety herein.

HDL Therapeutics of the disclosure include small molecules whose administration results in increased HDL levels. Exemplary small molecules include CETP inhibitors, e.g., torcetrapib, anacetrapib, evacetrapib, DEZ-001 (formerly TA-8995) and dalcetrapib, and those small molecules disclosed in U.S. Pat. Nos. 8,053,440; 5,783,600; 5,756,544; 5,750,569; 5,648,387; 8,642,653; 8,623,915; 8,497,301; 8,309,604; 8,153,690; 8,084,498; 8,067,466; 7,838,554; 7,812,199; 7,709,515; 7,705,177; 7,576,130; 7,335,799; 7,335,689; 7,304,093; 7,192,940; 7,119,221; 6,909,014; 6,831,105; 6,790,953; 6,713,507; 6,703,422; 6,699,910; 6,673,780; 6,646,170; 6,506,799; 6,459,003; and 6,410,802 all of which are incorporated by reference in their entirety herein.

The small molecules HDL Therapeutics of the disclosure also include CER-002 and CER-209:

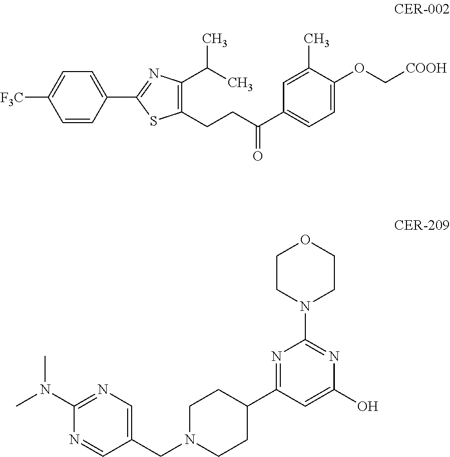 [SMILES: Cc1cc(C(=O)CCc2sc(-c3ccc(C(F)(F)F)cc3)nc2C(C)C)ccc1OCC(=O)O ; CN(C)c1ncc(CN2CCC(c3cc(O)nc(N4CCOCC4)n3)CC2)cn1]

The HDL Therapeutics may be formulated as pharmaceutical compositions. Pharmaceutical compositions contemplated by the disclosure comprise an HDL Therapeutic as the active ingredient in a pharmaceutically acceptable carrier suitable for administration and delivery to a subject.

Injectable compositions include sterile suspensions, solutions or emulsions of the active ingredient in aqueous or oily vehicles. The compositions can also comprise formulating agents, such as suspending, stabilizing and/or dispersing agent. In some embodiments, where the HDL Therapeutic is an HDL mimetic, the mimetic is formulated as an injectable composition comprising the HDL Therapeutic in phosphate buffered saline (10 mM sodium phosphate, 80 mg/mL sucrose, pH 8.2). The compositions for injection can be presented in unit dosage form, e.g., in ampules or in multidose containers, and can comprise added preservatives. For infusion, a composition can be supplied in an infusion bag made of material compatible with and HDL Therapeutic, such as ethylene vinyl acetate or any other compatible material known in the art.

Suitable dosage forms of HDL Therapeutics that are lipoprotein complexes or delipidated lipoproteins comprise an HDL Therapeutic at a final concentration of about 1 mg/mL to about 50 mg/mL of lipoprotein, and preferably about 5 mg/mL to about 15 mg/mL of lipoprotein. In a specific embodiment, the dosage form comprises an HDL Therapeutic at a final concentration of about 8 mg/mL to about 10 mg/mL Apolipoprotein A-I, preferably about 8 mg/mL.

### 6.4. HDL Markers

The present disclosure relates in part to utilization of HDL Markers that are downregulated by increasing dosing with HDL Therapeutics (whether by increased frequency, increase dose, or both). The HDL Markers are involved directly or indirectly in the removal of accumulated cholesterol or cholesteryl esters from monocytes, macrophages and mononuclear cells and include ATP-binding membrane cassette transporters A1 (ABCA1) and G1 (ABCG1) and the sterol regulatory element binding factor 1 gene (SREBP1), which plays an important role in the biosynthesis of fatty acids and cholesterol, and in lipid metabolism. In various embodiments, the methods of the disclosure assay for a single HDL Marker. In other embodiments, the methods of the disclosure assay for a plurality (e.g., two or three) HDL Markers. Exemplary combinations of HDL Markers that can be assayed for in the methods of the disclosure include ABCA1+ABCG1; ABCA1+SREBP1; ABCG1+SREBP1; and ABCA1+ABCG1+SREBP1, alone or in combination with additional markers. Methods of assaying HDL Markers are known in the art and exemplified below.

6.4.1. ABCA1

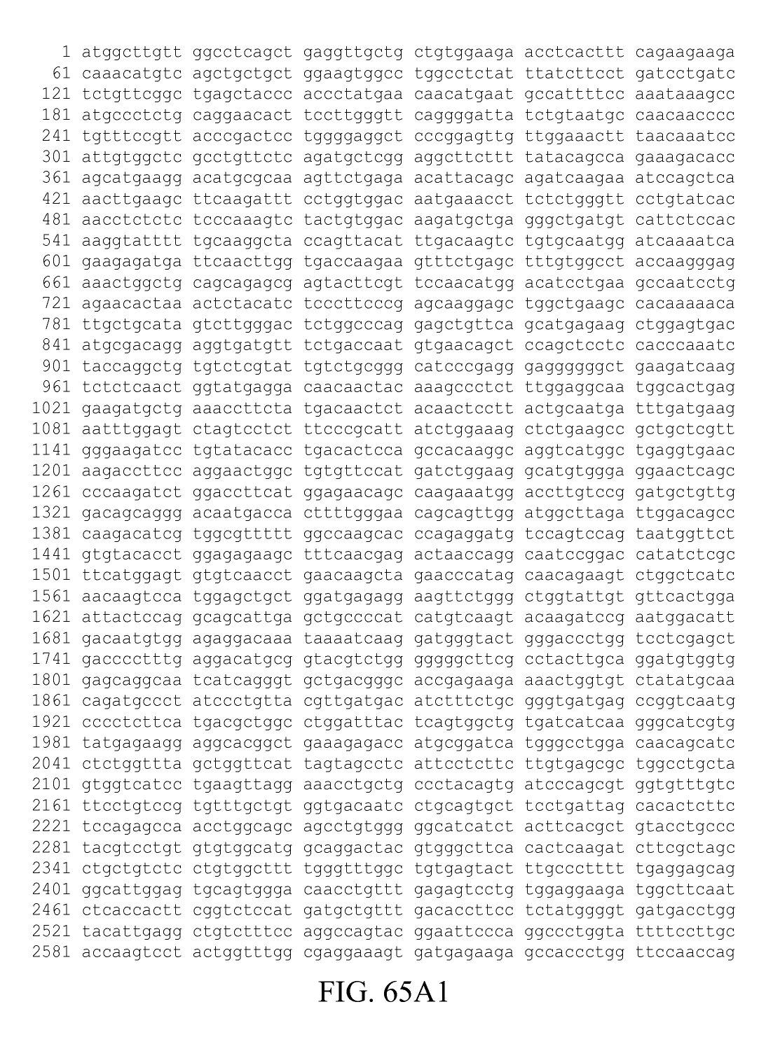

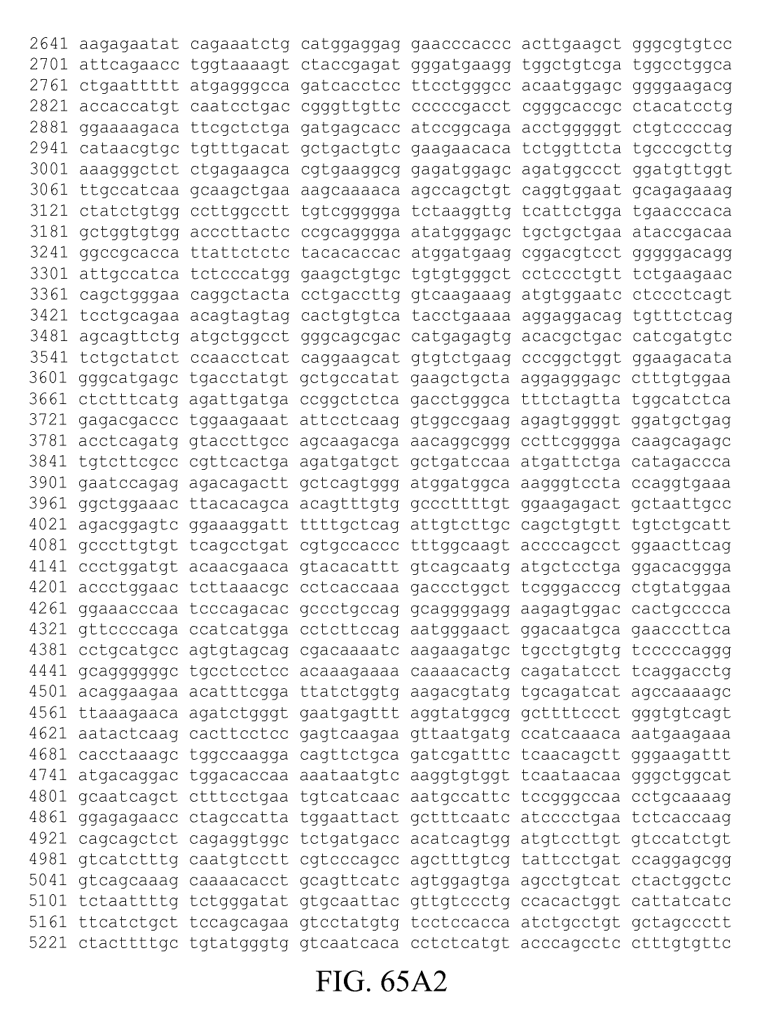

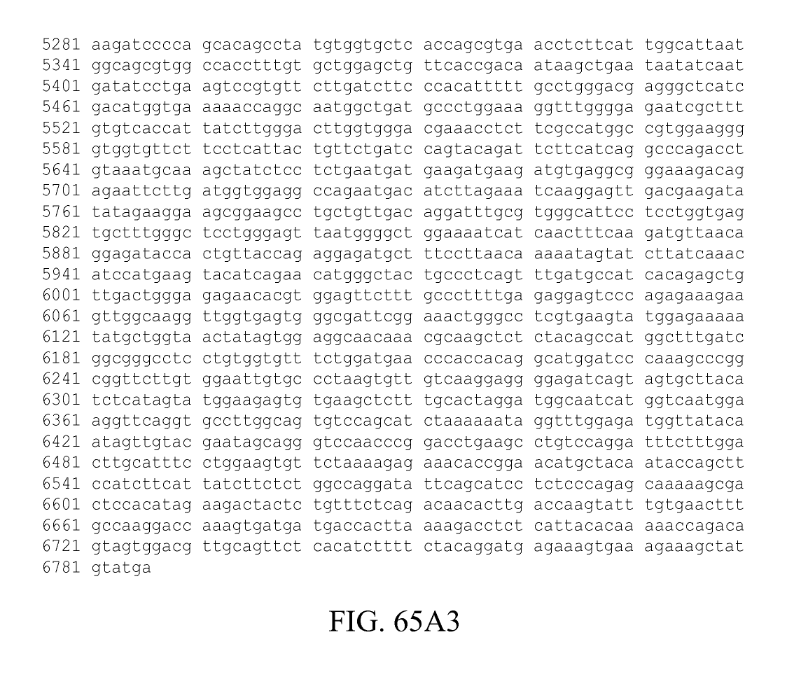

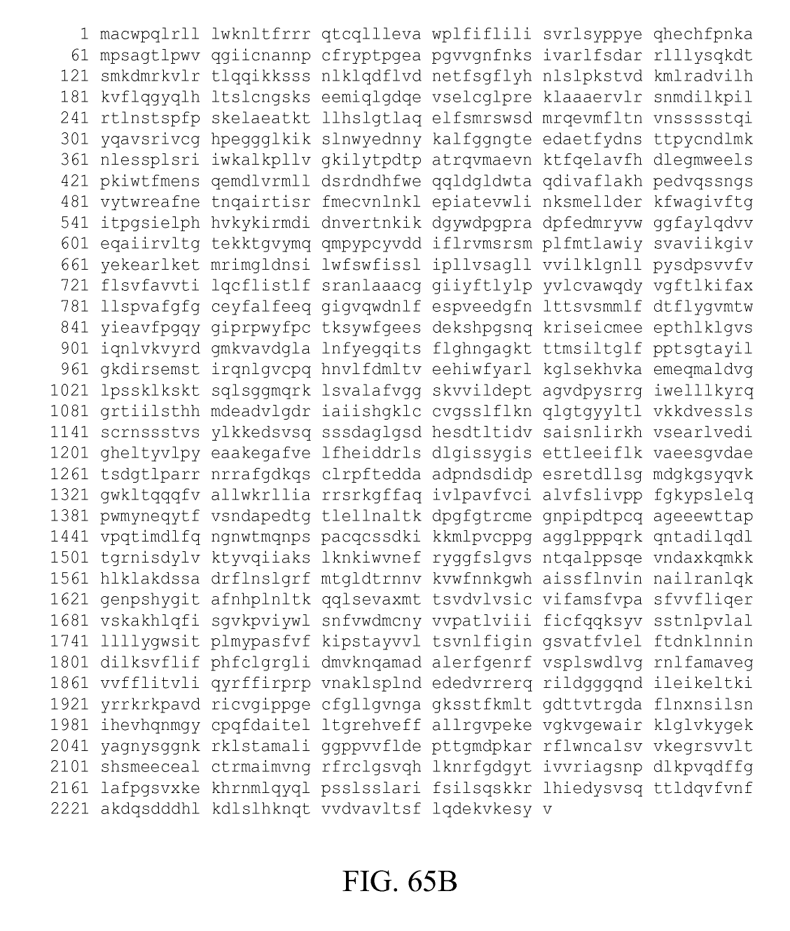

In various embodiments, the methods of the disclosure entail assaying for ABCAI expression levels and alterations in expression levels (e.g., in response to treatment with an HDL Therapeutic). An ABCA1 mRNA sequence whose expression levels can be assayed for is assigned accession no. AB055982.1, and an ABCA1 protein whose expression level can be assayed for is assigned accession no. AAF86276. These sequences are shown in FIGS. 65A1-65A3 and 65B, respectively.

Several RT-PCR and antibody detection systems have been developed which can be used to assay for ABCA1 expression according to the present methods, for example as described by Viñals et al., 2005, Cardiovascular Research 66:141-149; Sporstøl et al., 2007, BMC Mol Biol. 8:5; Genvigir et al., 2010, Pharmacogenomics 11(9):1235-46; Wang et al., 2007, Biochem Biophys Res Commun. 353(3):650-4; Holven et al., 2013, PLOS ONE 8(11):e78241; and Rubic & Lorenz, 2006, Cardiovascular Research 69:527-35.

6.4.2. ABCG1

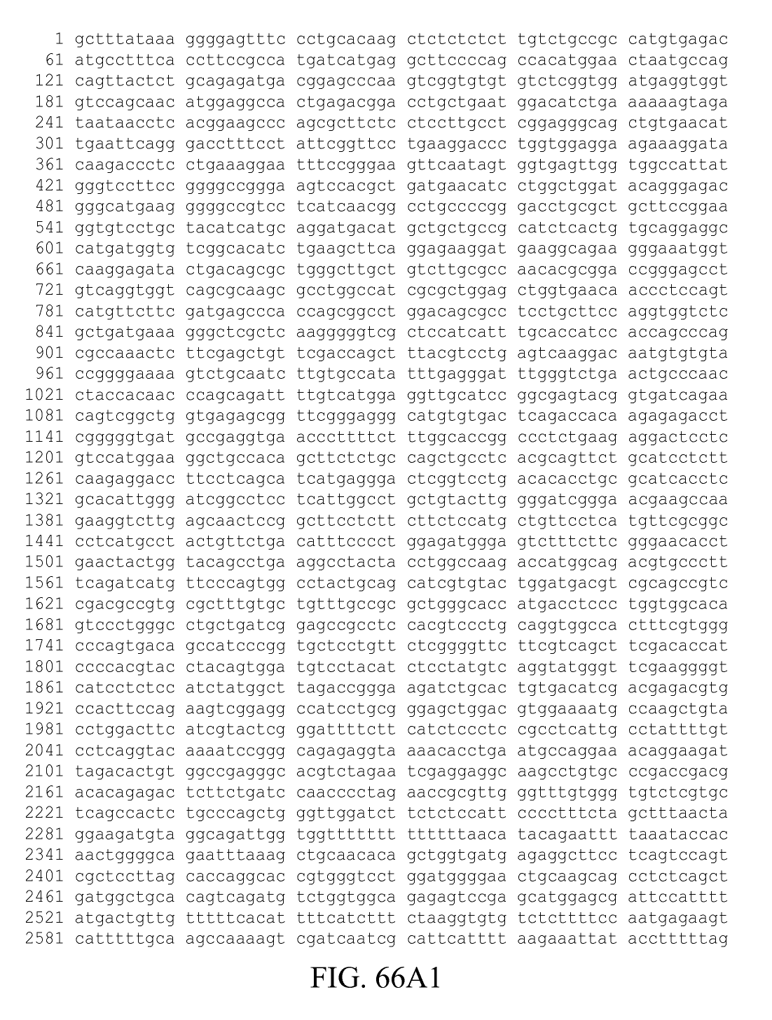

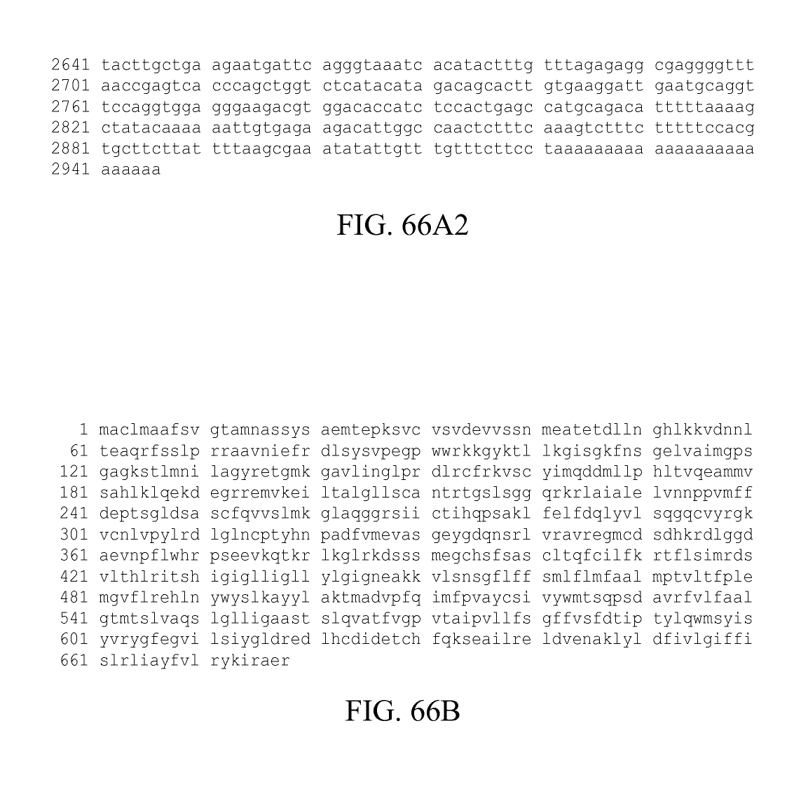

In various embodiments, the methods of the disclosure entail assaying for ABCGI expression levels and alterations in expression levels (e.g., in response to treatment with an HDL Therapeutic). An ABCG1 mRNA sequence whose expression levels can be assayed for is assigned accession no. NM_{—}207629.1, and an ABCG1 protein whose expression level can be assayed for is assigned accession no. P45844. These sequences are shown in FIGS. 66A1-66A2 and 66B, respectively.

Several RT-PCR and antibody detection systems have been developed which can be used to assay for ABCA1 expression according to the present methods, for example as described by Sporstøl et al., 2007, BMC Mol Biol. 8:5; Genvigir et al., 2010, Pharmacogenomics 11(9):1235-46; Wang et al., 2007, Biochem Biophys Res Commun. 353(3):650-4; Holven et al., 2013, PLOS ONE 8(11):e78241; and Rubic & Lorenz, 2006, Cardiovascular Research 69:527-35.

6.4.3. SREBP1

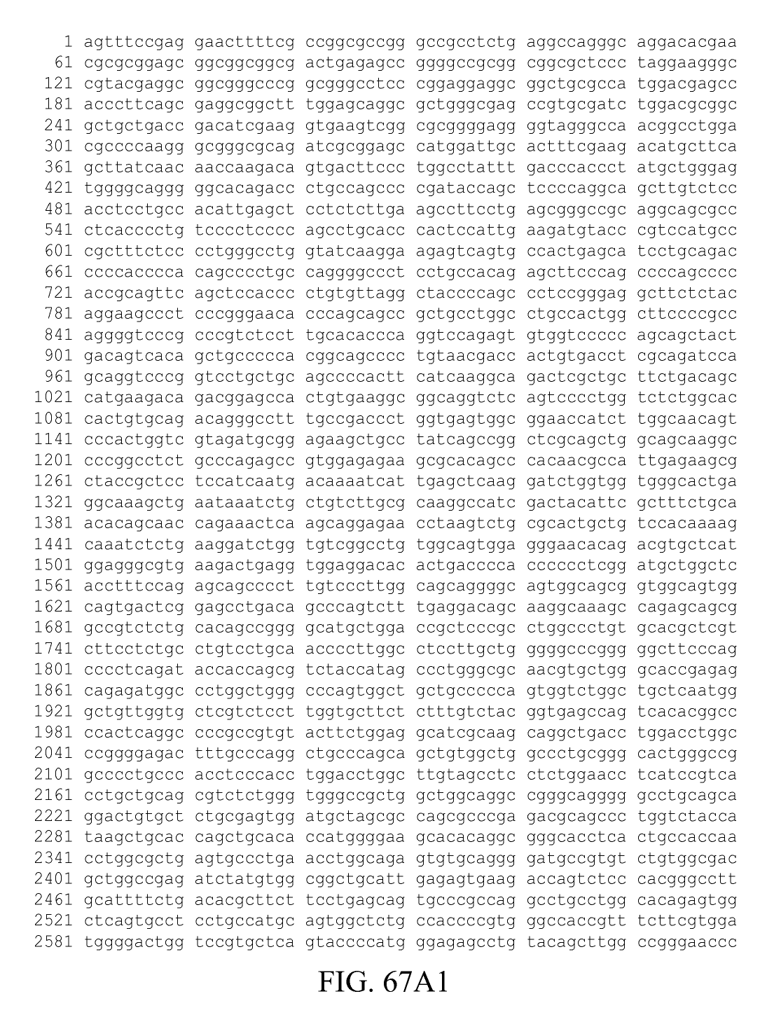

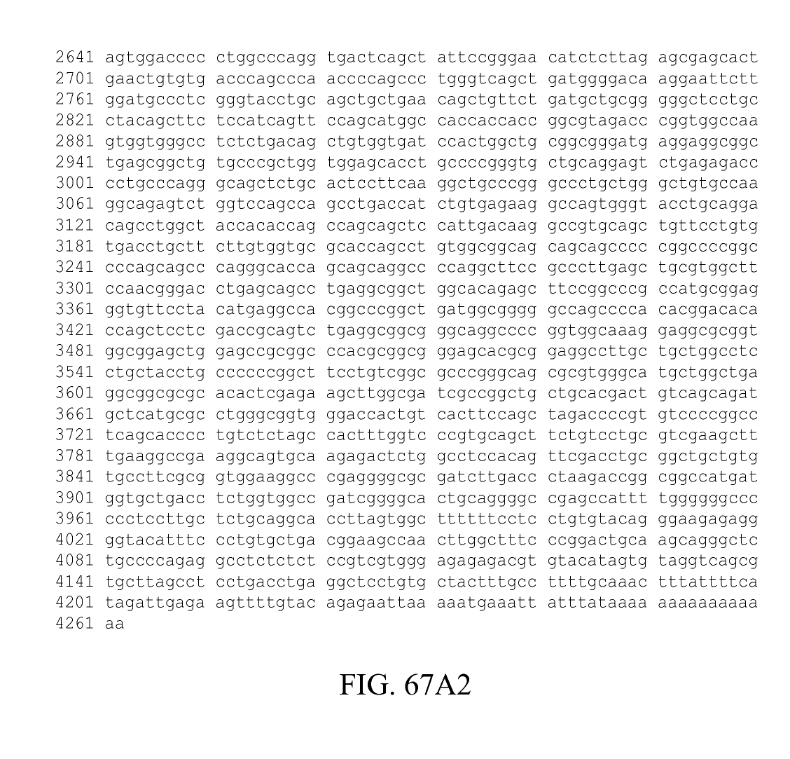

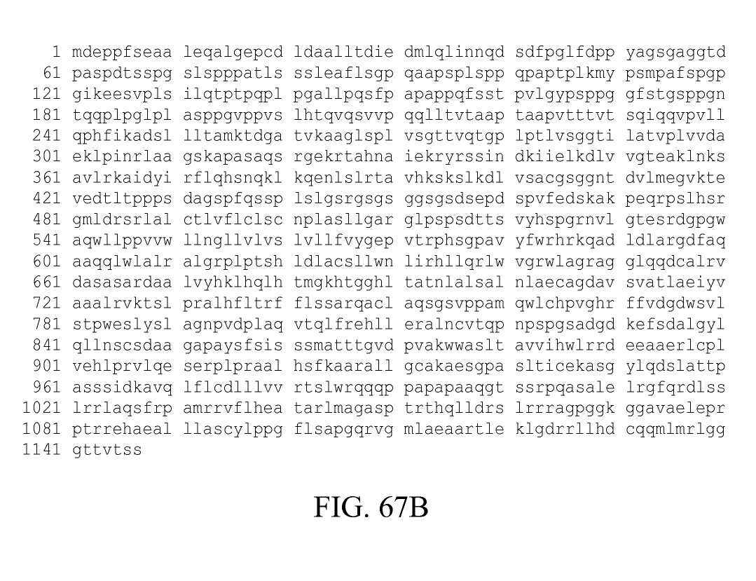

In various embodiments, the methods of the disclosure entail assaying for SREBP1 expression levels and alterations in expression levels (e.g., in response to treatment with an HDL Therapeutic). An SREBP1 mRNA sequence whose expression levels can be assayed for is assigned accession no. BC063281.1, and an SREBP1 protein whose expression level can be assayed for is assigned accession no. P36956. These sequences are shown in FIGS. 67A1-67A2 and 67B, respectively.

### 6.5. Monocytes

Monocytes are generated in the bone marrow to be released in the blood stream and also could also be in other biological fluids like cerebrospinal fluid, or lymph and give rise to different types of tissue-macrophages or dendritic cells after leaving the circulation. Monocytes, their progeny and immediate precursors in the bone marrow have also been named the “mono-nuclear phagocyte system” (MPS). They are derived from granulocyte/macrophage colony forming unit (CFU-GM) progenitors in the bone marrow that gives rise to monocytic and granulocytic cells.

Newly formed monocytes leave the bone marrow and migrate to the peripheral blood. Circulating monocytes can adhere to endothelial cells of the capillary vessels and are able to migrate into various tissues (van Furth et al., 1992, Production and Migration of Monocytes and Kinetics of Macrophages. In: van Furth R ed. Mononuclear Phagocytes. Dordrecht, The Netherlands: Kluwer Academic Publishers), where they can differentiate into macrophages or dendritic cells. Monocytes, macrophages, and dendritic cells are key cells in the initiation and progression of atherosclerosis. Under normal circumstances the endothelial monolayer in contact with flowing blood resists firm adhesion of monocytes. However, upon exposure to pro-inflammatory factors there is a steady increase in the expression of various leukocyte adhesion molecules in endothelial cells, which enables monocytes to adhere to the endothelial cell membranes (Libby, 2002, Nature 420:868-874). Once they have migrated, monocytes become tissue-resident macrophages, which in turn develop into lipid-loaded foam cells upon exposure to modified lipoproteins (Osterud and Bjorklid, 2003, Physiol. Rev. 83:1069-1112).

The diagnostic and dose optimization methods of the disclosure typically entail assaying monocytes or macrophages for HDL Marker expression prior to, during and/or following treatment with an HDL Therapeutic in order to identify optimal dosing on a patient level, a population level, in an animal model or in cell culture in vitro.

Methods of isolating peripheral blood monocytes are routine in the art. Such methods include density-gradient centrifugation (where the difference in the specific gravity of the cells is utilized for isolation), apheresis, attachment of monocytes to a plastic surface instrument such as a polystyrene flask, and cell sorting methods utilizing molecular markers.

Mononuclear cells can be isolated by a density-gradient centrifugation method.

Monocytes can be isolated through adherence of their adherence to a plastic (polystyrene) substrate, as the monocytes have a greater tendency to stick to plastic than other cells found in, for example, peripheral blood, such as lymphocytes and natural killer (NK) cells. Contaminating cells can be removed by vigorous washing of the substrate.

Monocytes can also be isolated using elutriation, a method by which a cell suspension is centrifuged in a chamber having a slope while flowing a buffer in an opposite direction from the centrifugation to form a particular cell layer.

The monocytes and macrophages are preferably isolated by the use of cell sorting methods (e.g., fluorescence activated cell sorting (FACS), magnetic-activated cell sorting (MACS), or flow cytometry) utilizing cell surface markers such as CD14 and CD16. Exemplary cell sorting methods and markers are disclosed in Mittar et al., August 2011 BD Biosciences publication entitled “Flow Cytometry and High-Content Imaging to Identify Markers of Monocyte-Macrophase Differentiation.”

### 6.6. Therapeutic Methods

The present disclosure provides methods for treating or preventing a Condition. In some embodiments, the method comprises administering an effective amount of an HDL Therapeutic to a subject in need thereof. The subject is preferably a mammal, most preferably a human. The methods of treatment can utilize doses (amounts and/or dosing schedules and/or infusion times) of HDL Therapeutics identified by the methods described herein and/or be accompanied by companion diagnostic assays utilizing HDL Markers as described herein to monitor the efficacy of the treatment.

Defects in ABCA1 result in the allelic disorders familial hypoalphalipoproteinemia (FHA) or the more severe disorder Tangier Disease (TD), that are characterized by greatly reduced level of HDL-C cholesterol in plasma, impaired cholesterol efflux, and a tendency to accumulate intracellular cholesterol ester. The present disclosure provides methods for treating such disorders.

The HDL Therapeutics and compositions described herein can be used for virtually every purpose HDL mimetics have been shown to be useful such as for treating or preventing ABCA1 related diseases or deficiency, treating or preventing ABCG1 related diseases of deficiency, and treating or preventing HDL deficiency, ApoA-I deficiency or LCAT deficiency. HDL Therapeutics may be used to treat or prevent diseases such as macular degeneration, stroke, atherosclerosis, acute coronary syndrome, endothelial dysfunction, accelerated atherosclerosis, graft atherosclerosis, ischemia, and transient ischemic attack.

HDL Therapeutics and compositions of the present disclosure are particularly useful to treat or prevent cardiovascular diseases, disorders, and/or associated conditions. Methods of treating or preventing a cardiovascular disease, disorder, and/or associated condition in a subject generally comprise administering to the subject a low (<15 mg/kg) dose or amount of an HDL Therapeutic or pharmaceutical composition described herein according to a regimen effective to treat or prevent the particular indication.

HDL Therapeutics are administered in an amount sufficient or effective to provide a therapeutic benefit. In the context of treating a cardiovascular disease, disorder, and/or associated condition, a therapeutic benefit can be inferred if one or more of the following occurs: an increase in cholesterol mobilization as compared to a baseline, a reduction in atherosclerotic plaque volume, an increase in the Percent Atheroma Volume (a measurement obtained by IVUS) (Nicholls et al., 2010, J Am Coll Cardiol 55:2399-407), an decrease in vessel wall thickness as measure by ultra sound imaging technique (Intimal Media Thickness) or by MRI (Duivenvoorden et al., 2009, Circ Cardiovasc Imaging. 2:235-242.), an increase in high density lipoprotein (HDL) fraction of free cholesterol as compared to a baseline level, without an increase in mean plasma triglyceride concentration or an increase above normal range of liver transaminase (or alanine aminotransferase) levels. A complete cure, while desirable, is not required for therapeutic benefit to exist.

In some embodiments, the HDL Therapeutic is a lipoprotein complex that is administered at a dose of about 1 mg/kg ApoA-I equivalents to about 15 mg/kg ApoA-I equivalents per injection. In some embodiments, the lipoprotein complex is administered at a dose of about 1 mg/kg, 2 mg/kg, or 3 mg/kg ApoA-I equivalents. In some embodiments, the lipoprotein complex is administered at a dose of about 6 mg/kg ApoA-I equivalents. In some embodiments, the lipoprotein complex is administered at a dose of about 8 mg/kg, 12 mg/kg or 15 mg/kg ApoA-I equivalents.

In some embodiments, the methods of treating or preventing a Condition described herein comprise a step of monitoring the treatment efficacy of the HDL Therapeutic, e.g., according to a method for monitoring the efficacy of an HDL Therapeutic described herein. The efficacy of the dose and/or dosing schedule of an HDL Therapeutic can be monitored by comparing the expression level of the one or more HDL Markers at two or more time points, for example, before administration of a dose of an HDL Therapeutic and after administration of the dose of the HDL Therapeutic. In some embodiments, the expression levels are measured 2-12 hours, 4-10 hours, 2-8 hours, 2-6 hours, 4-6 hours or 4-8 hours after administration of the dose of the HDL Therapeutic. In another embodiment, the expression levels of the one or more HDL Markers are measured before and after administration of an HDL Therapeutic according to a dosing schedule, e.g., a dosing schedule in which the HDL Therapeutic is administered every 2 days, every 3 days, every week day, or every two weeks.

The expression level can be a protein expression level or an mRNA expression level. In an embodiment, the expression level is a protein expression level determined using an antibody detection system, e.g., as described in Section 6.4. In another embodiment, the expression level is an mRNA expression level determined using RT-PCR. In an embodiment, expression levels of the one or more HDL Markers are measured in circulating monocytes, macrophages or mononuclear cells isolated according to any of the methods described in Section 6.5. The dose and/or dosing schedule of the HDL Therapeutic can be maintained or modified depending on whether or not the expression levels are reduced by more than the cutoff amounts for the one or more HDL Markers described in Section 6.2.

Subjects to be treated are individuals suffering from a cardiovascular disease, disorder, and/or associated condition. Non-limiting examples of such cardiovascular diseases, disorders and/or associated conditions that can be treated or prevented with the HDL Therapeutics and compositions described herein include, peripheral vascular disease, restenosis, atherosclerosis, and the myriad clinical manifestations of atherosclerosis, such as, for example, stroke, ischemic stroke, transient ischemic attack, myocardial infarction, acute coronary syndrome, angina pectoris, intermittent claudication, critical limb ischemia, valve stenosis, and atrial valve sclerosis. Subjects can be individuals with a prior incidence of acute coronary syndrome, such as a myocardial infarction (either with or without ST elevation) or unstable angina. The subject treated may be any animal, for example, a mammal, particularly a human.

In one embodiment, the methods encompass a method of treating or preventing a cardiovascular disease, accelerated atherosclerosis in a subject having an organ transplantation, such as heart transplantation (e.g., cardiac allograft vasculopathy (CAV)), kidney transplantation, or liver transplantation (Garcia-Garcia et al., 2010, European Heart Journal 3:2456-2469 at 2465, under “Cardiac Allograph Disease”). In some embodiments, the method comprises administering to a subject an HDL therapeutic or composition described herein.

In certain aspects, the methods encompass a method of treating or preventing a cardiovascular disease. In some embodiments, the method comprises administering to a subject an HDL Therapeutic or composition described herein in an amount that (a) does not alter a patient's baseline ApoA-I following administration and/or (b) is effective to achieve a serum level of free or complexed apolipoprotein higher than a baseline (initial) level prior to administration by about 5 mg/dL to 100 mg/dL approximately to two hours after administration and/or by about 5 mg/dL to 20 mg/dL approximately 24 hours after administration.

In another aspect, the methods encompass a method of treating or preventing a cardiovascular disease. In some embodiments, the method comprises administering to a subject an HDL Therapeutic or composition described herein in an amount effective to achieve a circulating plasma concentration of an HDL-cholesterol fraction for at least one day following administration that is at least about 10% higher than an initial HDL-cholesterol fraction prior to administration.

In another aspect, the methods encompass a method of treating or preventing a cardiovascular disease. In some embodiments, the method comprises administering to a subject an HDL Therapeutic or composition described herein in an amount effective to achieve a circulating plasma concentration of an HDL-cholesterol fraction that is between 30 and 300 mg/dL between 5 minutes and 1 day after administration.

In another aspect, the methods encompass a method of treating or preventing a cardiovascular disease. In some embodiments, the method comprises administering to a subject an HDL Therapeutic or composition described herein in an amount effective to achieve a circulating plasma concentration of cholesteryl esters that is between 30 and 300 mg/dL between 5 minutes and 1 day after administration.

In still another aspect, the methods encompass a method of treating or preventing a cardiovascular disease. In some embodiments, the method comprises administering to a subject an HDL Therapeutic or composition described herein in an amount effective to achieve an increase in fecal cholesterol excretion for at least one day following administration that is at least about 10% above a baseline (initial) level prior to administration.

The HDL Therapeutics or compositions described herein can be used alone or in combination therapy with other drugs used to treat or prevent the foregoing conditions. Such therapies include, but are not limited to simultaneous or sequential administration of the drugs involved. For example, in the treatment of dyslipidemia, hypercholesterolemia, such as familial hypercholesterolemia (homozygous or heterozygous) or atherosclerosis, HDL Therapeutics can be administered with any one or more of the cholesterol lowering therapies currently in use; e.g., bile-acid resins, niacin, statins, inhibitors of cholesterol absorption and/or fibrates. Such a combined regimen may produce particularly beneficial therapeutic effects since each drug acts on a different target in cholesterol synthesis and transport; i.e., bile-acid resins affect cholesterol recycling, the chylomicron and LDL population; niacin primarily affects the VLDL and LDL population; the statins inhibit cholesterol synthesis, decreasing the LDL population (and perhaps increasing LDL receptor expression); whereas the HDL Therapeutics described herein affect RCT, increase HDL, and promote cholesterol efflux.

In another embodiment, the HDL Therapeutics or compositions described herein may be used in conjunction with fibrates to treat or prevent coronary heart disease; coronary artery disease; cardiovascular disease, restenosis, vascular or perivascular diseases; atherosclerosis (including treatment and prevention of atherosclerosis).

The HDL Therapeutics or compositions described herein can be administered in dosages that increase the small HDL fraction, for example, the pre-beta, pre-gamma and pre-beta-like HDL fraction, the alpha HDL fraction, the HDL₃ and/or the HDL₂ fraction. In some embodiments, the dosages are effective to achieve atherosclerotic plaque reduction as measured by, for example, imaging techniques such as magnetic resonance imaging (MRI) or intravascular ultrasound (IVUS). Parameters to follow by IVUS include, but are not limited to, change in percent atheroma volume from baseline and change in total atheroma volume. Parameters to follow by MRI include, but are not limited to, those for IVUS and lipid composition and calcification of the plaque.

The plaque regression could be measured using the patient as its own control (time zero versus time t at the end of the last infusion, or within weeks after the last infusion, or within 3 months, 6 months, or 1 year after the start of therapy.

Administration can best be achieved by parenteral routes of administration, including intravenous (IV), intramuscular (IM), intradermal, subcutaneous (SC), and intraperitoneal (IP) injections. In certain embodiments, administration is by a perfusor, an infiltrator or a catheter. In some embodiments, the HDL Therapeutics are administered by injection, by a subcutaneously implantable pump or by a depot preparation, in amounts that achieve a circulating serum concentration equal to that obtained through parenteral administration. The HDL Therapeutics could also be absorbed in, for example, a stent or other device.

Administration can be achieved through a variety of different treatment regimens. For example, several intravenous injections can be administered periodically during a single day, with the cumulative total volume of the injections not reaching the daily toxic dose. The methods comprise administering the HDL Therapeutic at an interval of 2, 3, 4, 5, 6, 7, 8, 9, 10, 11, or 12 days. In some embodiments, the HDL Therapeutic is administered at an interval of once a week, twice a week, three times a week or more.

The methods can further comprise administering the HDL Therapeutic 4, 5, 6, 7, 8, 9, 10, 11, or 12 times or more at any of the intervals described above. In subjects suffering from a familial primary hypoalphalipoproteinemia, the HDL Therapeutic can be administered for months, years or indefinitely.

For example, in one embodiment, the HDL Therapeutic is administered six times, with an interval of 1 week between each administration. In some embodiments, administration could be done as a series of injections and then stopped for 6 months to 1 year, and then another series started. Maintenance series of injections could then be administered every year or every 3 to 5 years. The series of injections could be done over a day (perfusion to maintain a specified plasma level of complexes), several days (e.g., four injections over a period of eight days) or several weeks (e.g., four injections over a period of four weeks), and then restarted after six months to a year. For chronic conditions, administration could be carried out on an ongoing basis. Optionally, the methods can be preceded by an induction phase, when the HDL Therapeutic is administered more frequently.

In another embodiment, treatment with an HDL Therapeutic can be initiated according to an induction dosing regimen, followed by a maintenance regimen in which the dose and/or frequency of administration are reduced. For example, an induction regimen can entail administering an HDL Therapeutic twice, three or four times a week. Where the HDL Therapeutic is a lipoprotein complex such as CER-001, the induction dose can range between 4-15 mg/kg on a protein basis (e.g., 4, 5, 6, 7, 8, 9, 10, 12 or 15 mg/kg). A maintenance regimen can entail administering the HDL Therapeutic once, twice or three times a week. Where the HDL Therapeutic is a lipoprotein complex such as CER-001, the maintenance dose can range 0.5-8 mg/kg on a protein basis (e.g., 0.5, 1, 2, 3, 4, 5, 6, 7 or 8 mg/kg). Induction dosing schedules are particularly suitable for subjects suffering from familial primary hypoalphalipoproteinemia. An illustrative dosing schedule is described in Example 4.

In yet another alternative, an escalating dose can be administered, starting with about 1 to 12 doses at a dose between 1 mg/kg and 8 mg/kg per administration, then followed by repeated doses of between 4 mg/kg and 15 mg/kg per administration. Depending on the needs of the patient, administration can be by slow infusion with a duration of more than one hour, by rapid infusion of one hour or less, or by a single bolus injection. The doses can be administered once, twice, three times a week or more.

Toxicity and therapeutic efficacy of the various HDL Therapeutics can be determined using standard pharmaceutical procedures in cell culture or experimental animals for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. HDL Therapeutics that exhibit large therapeutic indices are preferred. Non-limiting examples of parameters that can be followed include liver function transaminases (no more than 2× normal baseline levels). This is an indication that too much cholesterol is brought to the liver and cannot assimilate such an amount. The effect on red blood cells could also be monitored, as mobilization of cholesterol from red blood cells causes them to become fragile, or affect their shape. The downregulation of ABCA1, ABCG1 or the HDL markers described herein could also be monitored.

Patients can be treated from a few days to several weeks before a medical act (e.g., preventive treatment), or during or after a medical act. Administration can be concomitant to or contemporaneous with another invasive therapy, such as, angioplasty, carotid ablation, rotoblader or organ transplant (e.g., heart, kidney, liver, etc.).

In certain embodiments, an HDL Therapeutics is administered to a patient whose cholesterol synthesis is controlled by a statin or a cholesterol synthesis inhibitor (such as but not limited to PCSK9 inhibitor). In other embodiments, an HDAL Therapeutic is administered to a patient undergoing treatment with a binding resin, e.g., a semi-synthetic resin such as cholestyramine, or with a fiber, e.g., plant fiber, to trap bile salts and cholesterol, to increase bile acid excretion and lower blood cholesterol concentrations.

### 7. EXAMPLE 1

### Dose Response Analysis of CER-001

7.1. Chi Square Clinical Trials

CER-001 is an engineered recombinant human apolipoprotein A-I High Density Lipoprotein (HDL) with a negative charge that mimics biological properties of natural HDL when injected intravenously. CER-001, described as “Formula H” in Examples 3 and 4 of WO2012/109162, incorporated by reference in its entirety herein, is composed of recombinant human apolipoprotein A-I and phospholipid, containing Sphingomyelin (Sph) and dipalmitoyl phosphatidylglycerol (DPPG). The protein-to-phospholipid ratio is 1:2.7 and contains 97% Sph and 3% DPPG.

As described in Example 8 of WO2012/109162, a phase I study of CER-001 in healthy volunteers at single IV doses of 0.25, 0.75, 2, 5, 10, 30 and 45 mg/kg showed that the complex was well-tolerated and increased cholesterol mobilization with increasing doses and, at levels of greater than 15 mg/kg, a transient increase of triglycerides was observed.

On the basis of the phase I study, a phase II study entitled “Can HDL Infusions Significantly QUicken Artherosclerosis REgression” (“CHI SQUARE”) was initiated. 504 subjects presenting with acute chest pain or other angina equivalent symptoms, indicative of a diagnosis of ST segment elevation myocardial infarction, non-ST elevation myocardial infarction or unstable angina were enrolled. To be eligible, subjects must have angiographic evidence of coronary artery disease as defined by at least one lesion in any of the three major native coronary arteries that has >20% reduction in lumen diameter by angiographic visual estimation or prior history of percutaneous coronary intervention (“PCI”). The target vessel for PCI was not the target coronary artery for research IVUS, and any vessel with previous PCI could not be used as the target coronary artery.

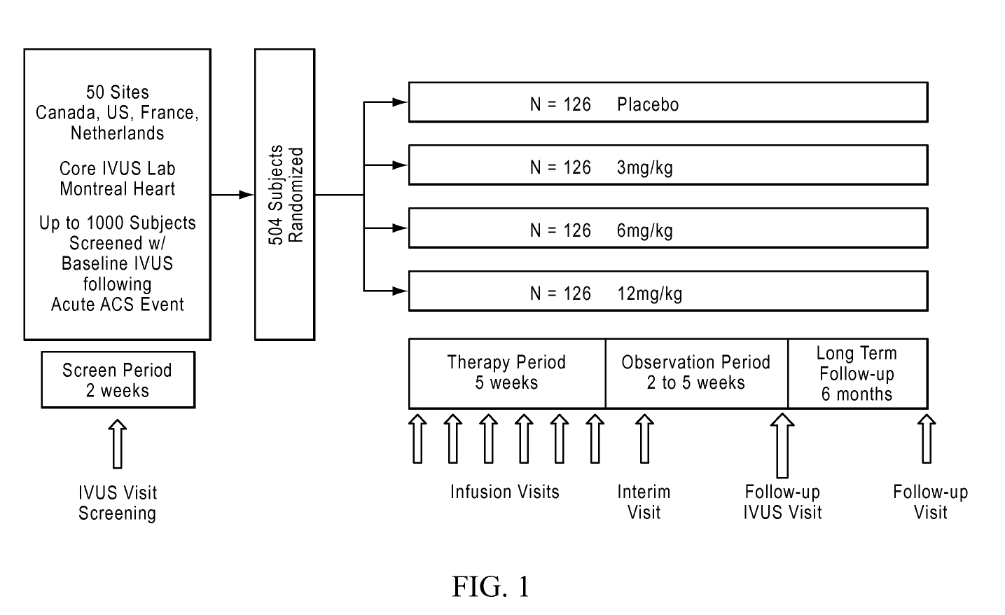

The study design is illustrated in FIG. 1. The primary endpoint was the nominal change in total plaque volume in a 30 mm segment of the target coronary artery assessed by 3 dimensional IVUS (intra-vascular ultrasound). The key secondary endpoints were % change in plaque volume and change in % atheroma volume in the target 30 mm segment, the change in total vessel volume in the target 30 mm segment, and changes in plaque, lumen and total vessel volumes from baseline in the least and most diseased 5 mm segments. Morbidity and mortality were exploratory endpoints.

7.2. Results

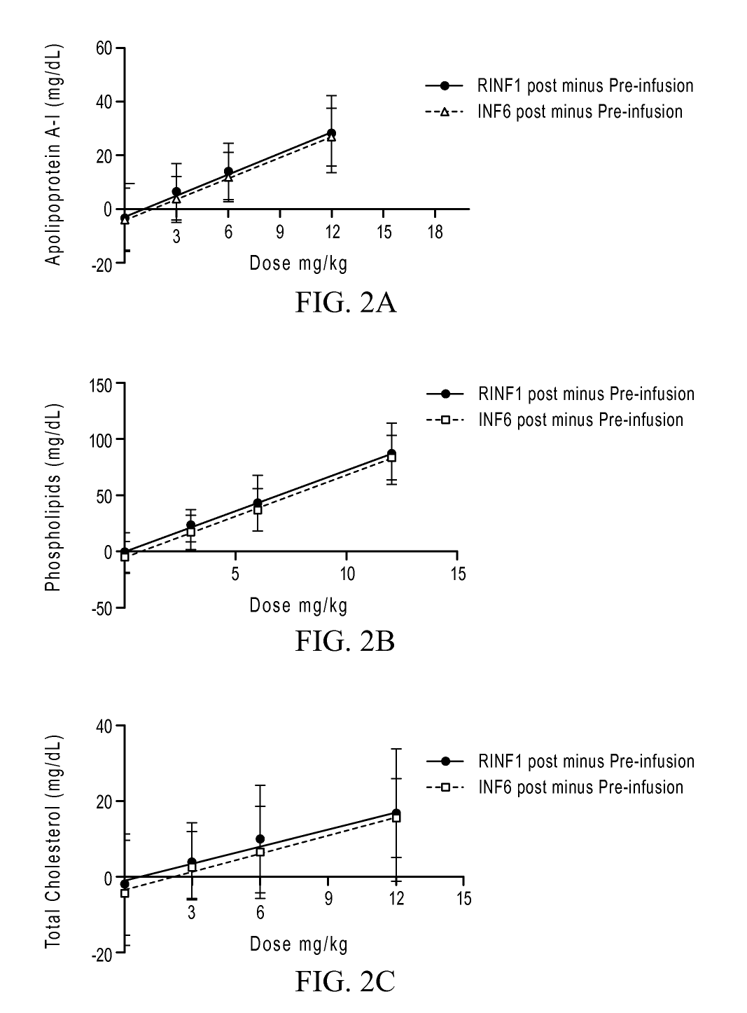

Following IV CER-001 administration, apolipoprotein A-I increases in a dose-dependent manner (Infusion 1) and at a magnitude consistent with that predicted from Phase I. This effect was preserved at Infusion 6, indicating no attenuation of efficacy over time. See FIG. 2A.

Phospholipids also increase in a dose dependent manner (Infusion 1) and at a magnitude consistent with that predicted from Phase I. This effect was preserved at Infusion 6, indicating no attenuation of efficacy over time. See FIG. 2B. The slope ratio of phospholipids and ApoA-I dose response curves is 2.8, consistent with the phospholipid to protein ratio in CER-001.

Plasma total cholesterol increases in a dose-dependent manner (Infusion 1) and at a magnitude consistent with that predicted from Phase I. This effect was preserved at Infusion 6, indicating no attenuation of efficacy over time. See FIG. 2C. These data show that the potency of CER-001 at 3 mg/kg is comparable to ETC-216 at 15 mg/kg.

CER-001 was well-tolerated overall at doses of 3, 6, and 12 mg/kg with no apparent dose-related toxicities in laboratory parameters.

7.2.1. IVUS—First Approach

Mean total atheroma volume at baseline was 155.24±67.99 mm³. The adjusted means for change in total atheroma volume were −2.71, −3.13, −1.50 and −3.05 mm³ in the placebo, CER-001 3 mg/kg, CER-001 6 mg/kg and CER-001 12 mg/kg groups, respectively (p=0.81 for the prespecified primary analysis of 12 mg/kg versus placebo). There were also no differences compared to placebo for the CER-001 6 mg/kg (nominal p=0.45) and 3 mg/kg (nominal p=0.77) groups. The change in percent atheroma volume was similar among all study groups (0.02, −0.02, 0.01 and 0.19% in the placebo, CER-001 3 mg/kg (p=0.86), CER-001 6 mg/kg (p=0.95) and CER-001 12 mg/kg (p=0.53) groups (nominal p-values versus placebo).

In contrast to the “walk-along” approach discussed below, frame pairs were individually selected for optimum readability. Frames were selected based upon absence of echogenicity (calcium) and side branches. A maximum of 31 frames were selected over a 30 mm segment, excluding the benefit of pull-backs longer than 30 mm. No pre-defined criteria were used to select the 31 frames for inclusion in analysis set when >31 frames available.

˜60% of paired image sets were clustered at 31 frames and ˜16% of paired image sets clustered at 16 frames.

“Clustering” at 16-frame image sets is suggestive that frames were selected at intervals smaller than 1 mm (i.e., as low as 0.3 mm) in order to qualify the image set for analysis.

“Clustering” at 31-frame image sets is suggestive that <1 mm intervals may have also been used to maximize number of image pairs to 31.

More details of the analysis are described in Tardif et al., 2014, Eur. Heart Journal, first published online Apr. 29, 2014 doi:10.1093/eurheartj/ehu171), incorporated by reference herein in its entirety.

Under this approach, at 126 patients/treatment arm, CHI SQUARE was underpowered to show significance for cardiovascular events (approximately 5000 patients/arm would have been required).

7.2.2. IVUS Analysis—Second Approach

A post-hoc analysis of the IVUS data was performed by the South Australian Health & Medical Research Institute (SAHMRI).

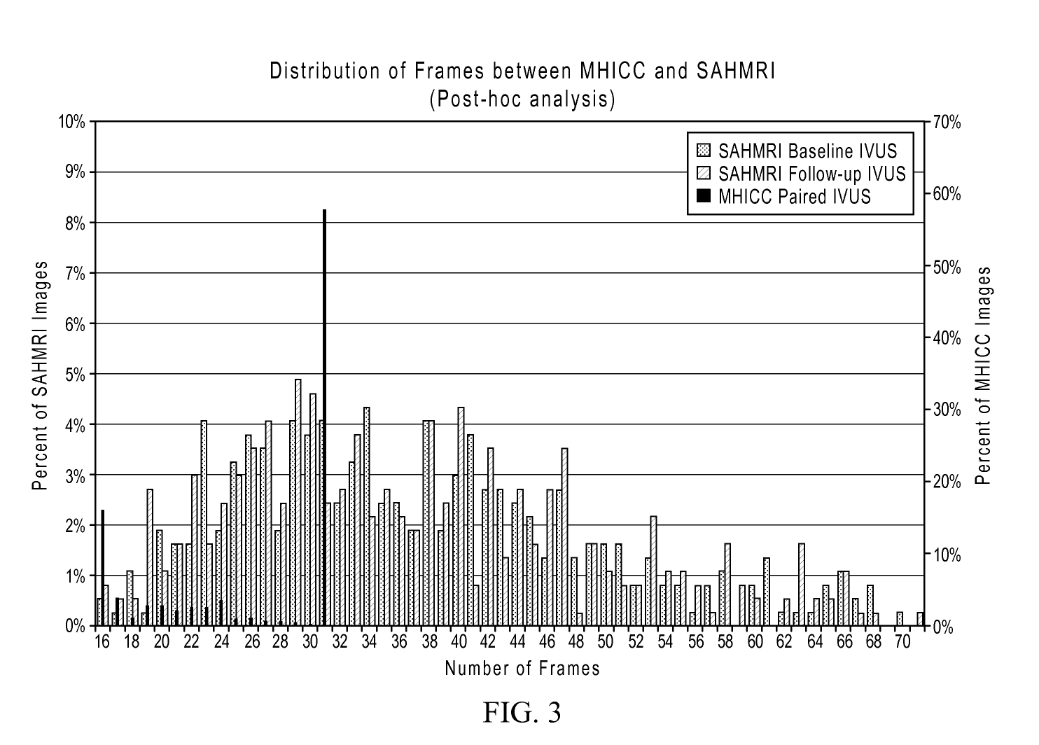

In this case there were similar frame-counts between baseline and follow-up. The number of frames selected were normally distributed (FIG. 3).

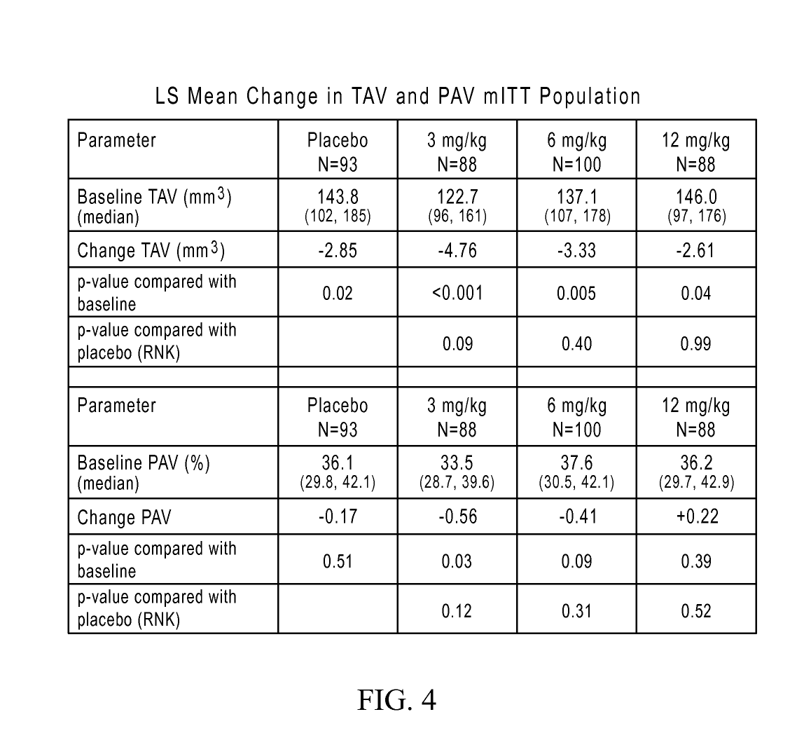

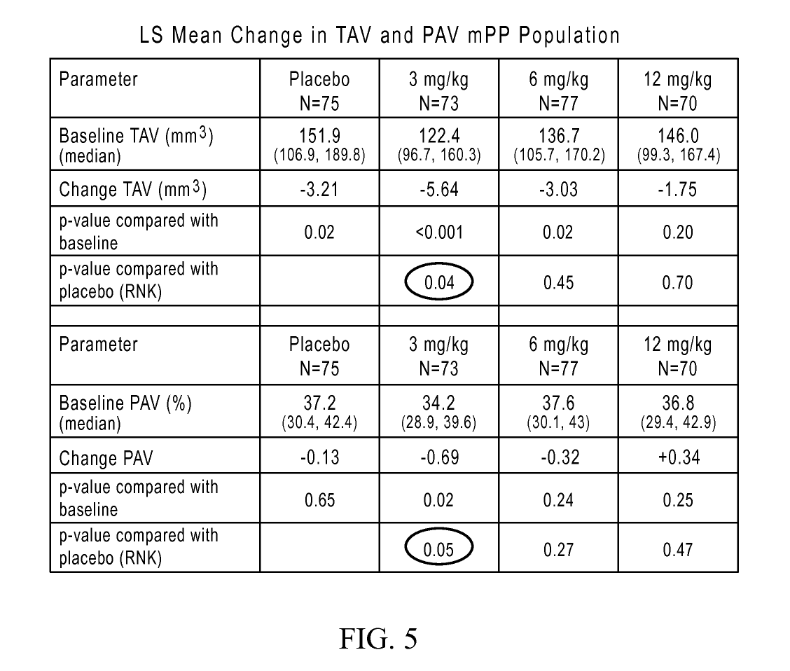

The analysis demonstrated a statistically significant and comparable magnitude of reduction in PAV and TAV versus baseline compared to prior HDL mimetics (FIG. 4). Although the study did not reach the primary endpoint in the mITT population, in the modified Per Protocol (mPP) population, the 3 mg/kg dose did achieve nominal statistical significance versus placebo in both TAV and PAV (FIG. 5).

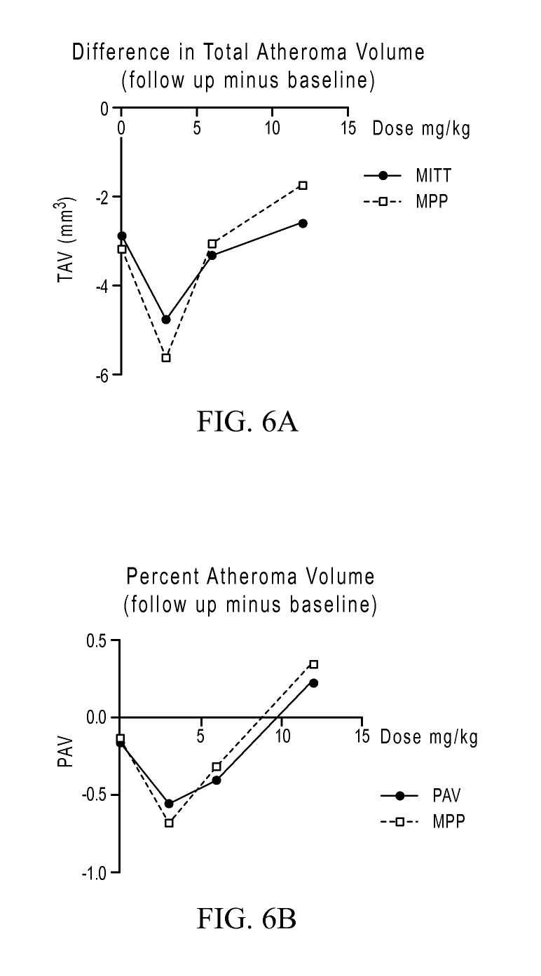

As can be seen in FIGS. 6A-6B, the results of the SAHMRI analyses are consistent with an inverted U-shaped dose-effect curve for CER-001 in humans.

In patients for which baseline PAV was equal to or greater than 30, the lowest dose of 3 mg/kg obtained statistical significance versus placebo for the change in total atherosclerotic volume (TAV) and the change in PAV for all patients (mITT), as shown in Table 1.

TABLE 1

| | Test for Normality | | LS Means and p-values from ANCOVA Modeling | | | | | | |
|---|---|---|---|---|---|---|---|---|---|
| | | p- | Placebo | 3 mg/kg | p- | 6 mg/kg | p- | 12 mg/kg | p- |
| Parameter | W | value | (n = 69) | (n = 58) | value^{†} | (n = 78) | value^{†} | (n = 66) | value^{†} |
| PAV | 0.927 | <0.0001 | −0.259 | −0.963 | 0.131 | −0.619 | 0.404 | +0.177 | 0.331 |
| | | | | | (P) | | (P) | | (P) |
| | | | | | 0.038* | | 0.287 | | 0.587 |
| | | | | | (NP) | | (NP) | | (NP) |
| TAV | 0.986 | 0.009 | −2.744 | −6.258 | 0.124 | −3.429 | 0.744 | −2.726 | 0.994 |
| | | | | | (P) | | (P) | | (P) |
| | | | | | 0.035* | | 0.500 | | 0.927 |
| | | | | | (NP) | | (NP) | | (NP) |

^{†}Parametric testing from ANCOVA model using baseline value as a covariate; nonparametric testing from ANCOVA model on ranked data using actual baseline value as a covariate. Nonparametric results should be used when the Shapiro-Wilk test has a p-value <0.5.
*Statistically significant result

The dose response in the subpopulation of patients with PAV 30 at baseline followed the same pattern as in the total population, but with an even more pronounced change in TAV and PAV at the 3 mg/kg dose.

### 8. EXAMPLE 2

### Regulation of Genes Implicated in Reverse Lipid Transport after Treatment with CER-001 or HDL₃

8.1. Introduction

The objective of studies A-P was to determine the regulation of the genes implicated in reverse lipid transport (RLT) after the treatment of mouse macrophages (J774) with CER-001, HDL₃ and ApoA-I. Reverse cholesterol transport (RCT) is the pathway by which peripheral cells release accumulated cholesterol to an extracellular acceptor such as high-density lipoprotein (HDL) which then mediates cholesterol delivery to the liver for excretion, thus preventing atherosclerosis. One approach to study cholesterol efflux is to label macrophages with [³H]-cholesterol-oxidized-LDL and measure cholesterol release from these cells in the presence of acceptor molecules. ABCA1, ABCG1 and SR-BI are membrane proteins implicated in cholesterol efflux.

8.2. Materials

The materials used for these studies included CER-001 (a charged lipoprotein complex with 1:2.7 protein to total lipid ratio, 97% egg sphingomyelin/3% DPPG) at a concentration of 13.5 mg/mL ApoA-I), purified human HDL₃ and purified ApoA-I. The materials were stored at ca. −20° C.

HDL₃ lipoprotein fractions were prepared from human plasma according to the process described in Section 8.3.1. Briefly, VLDL, IDL and LDL fractions were first removed with a KBr gradient (d<1.055) and sequential ultracentrifugations (3 times 100,000×g for 24 h). The LDL fraction was saved for future use in the cholesterol efflux experiment. The HDL₃ fraction was then isolated from a KBr gradient (d=1.19)−100,000×g for 40 h. The lipoprotein fractions were extensively dialyzed against phosphate buffered saline (PBS) before utilization in experiments.

8.3. Protocols

8.3.1. Separation of Plasmatic Lipoproteins

Reception of the Plasma.

Measure of the volume of fresh plasma (not frozen). Add the additives at the final concentrations: EDTA: 0.1% (w/v), NaN₃: 0.01% (w/v). Centrifuge the plasma at 20 000 rpm, 4° C., 20 min. Remove cell debris and possible chylomicrons to afford a clear plasma.

Lipoprotein Isolation.

The lipoproteins are obtained by sequential flotation ultracentrifugation in KBr solution (VLDL, d=1.006 g/mL; LDL, 1.006<d<1.063 g/mL). HDL₂ were first isolated (110,000×g for 40 h) at d=1.125 g/mL followed by HDL₃ (110,000×g for 40 h) at d=1.19 g/mL. Before use, the lipoproteins are extensively dialyzed against phosphate-buffered saline. The volume of the saline solution is serum volume—7%, corresponding to the volume of hydrated proteins.

8.3.2. RNA Extraction

Step 1—Homogenization:

Homogenize tissue sample (50 mg) or cultured cells (1 well of 6-well plate) in 1 ml TRI Reagent®. Incubate the homogenate for 5 min at room temperature in a 1.5 ml RNase-free tube. For tissue sample, centrifuge at 12,000×g for 10 min at 4° C. and transfer the supernatant to a new tube. Note: not necessary for cultured cell sample.

Step 2—RNA Extraction:

Add 100 μl of Bromo Chloropropane (BCP) to 1 ml of homogenate and mix well (vortex for 15 s). Incubate for 5 min at room temperature. Centrifuge at 12,000×g for 10 min at 4° C. Transfer 400 μl aqueous upper phase to a new 1.5 ml RNase-free tube.

Step 3—Final RNA Purification:

Add 200 μl of 100% ethanol and mix immediately (vortex for 5 s). Pass the sample through a filter cartridge by centrifugation at 12,000×g for 30 s. Wash the filter twice with 500 μl of Wash Solution (12,000×g for 30 s). Centrifuge for 30 s more to remove residual Wash Solution. Transfer the filter cartridge to a new collection tube. Add 50-100 μl of Elution Buffer to the filter column, incubate for 2 min at room temperature and centrifuge at 12,000×g for 30 s to elute RNA from the filter. Store the recovered RNA at −80° C.

Step 4—Determine the RNA Concentration:

The concentration of an RNA solution is determined by measuring its absorbance at 260 nm on a Nanodrop Spectrophotometer on 1.5 μl of sample. To assess the RNA quality, an analysis with the Agilent 2100 bioanalyzer can be made as described in Section 8.3.3.

8.3.3. RNA Quality Determination with Agilent Bioanalyzer

Allow all reagents to equilibrate at room temperature for 30 minutes before use. Protect the dye concentrate from light while bringing it to room temperature.

Step 1—Prepare the Gel:

Place 550 μl of Agilent RNA 6000 Nano gel matrix into a spin filter. Centrifuge for 10 minutes at 1500×g. Aliquot 65 μl of filtered gel into 0.5 ml RNase-free microfuge tubes that are included in the kit. Use filtered gel within a month.

Step 2—Prepare the Gel-Dye Mix:

Vortex RNA 6000 Nano dye concentrate for 10 seconds and spin down. Add 1 μl of RNA 6000 Nano dye concentrate to a 65 μl aliquot of filtered gel. Cap the tube, vortex thoroughly and visually inspect proper mixing of gel and dye. Spin tube for 10 minutes at room temperature at 13,000×g. Use prepared gel-dye mix within one day. Always re-spin the gel-dye mix at 13,000×g for 10 minutes before each use.

Step 3—Load the Gel-Dye Mix:

Before loading the gel-dye mix, make sure that the base plate of the chip priming station is in position (C) and the adjustable clip is set to the top position. Put a new RNA 6000 Nano chip on the chip priming station. Pipette 9 μl of the gel-dye mix at the bottom of the surrounded G well. Set the timer to 30 seconds, make sure that the plunger is positioned at 1 ml and then close the chip priming station. The lock of the latch will click when the Priming Station is closed correctly. Press the plunger of the syringe down until it is held by the clip. Wait for exactly 30 seconds and then release the plunger with the clip release mechanism. Wait for 5 seconds, then slowly pull back the plunger to the 1 ml position. Open the chip priming station slowly. Pipette 9 μl of the gel-dye mix in each of the two G wells.

Step 4—Load the Agilent RNA 6000 Nano Marker:

Pipette 5 μl of the RNA 6000 Nano marker into the well marked with the ladder symbol and each of the 12 sample wells.

Step 5—Load the Ladder and Samples:

Before use, thaw ladder aliquots and RNA samples and keep them on ice. To minimize secondary structure, heat denature (70° C., 2 minutes) the samples before loading on the chip. Pipette 1 μl of prepared ladder into the well marked with the ladder symbol. Pipette 1 μl of each sample into each of the 12 sample wells. Pipette 1 μl of RNA 600 Nano Marker in each unused sample well. Place the chip horizontally in the adapter of the IKA vortex mixer and vortex for 1 min at 2 400 rpm. Run the chip in the Agilent 2100 bioanalyzer within 5 min.

Step 6—Start the Analysis of the Chip:

In the Instrument context, select the appropriate assay from the Assay menu (for example: Assay RNA eucaryotes) and select the COM Port 1. Accept the current File Prefix or modify it. Data will be saved automatically to a file with a name using this prefix. At this time, the file storage location and the number of samples that will be analyzed can be customized. Click the Start button in the upper right of the window to start the chip run. To enter sample information, such as sample names and comments, select the Data File link that is highlighted in blue or go to the Assay context and select the Chip Summary tab. Complete the sample name table. After the chip run is finished, remove the chip immediately from the receptacle.

8.3.4. Reverse Transcription

Prepare the RT reaction mix using the high capacity RNA to cDNA kit (Applied Biosystems cat. No. 4387406) before preparing the reaction tubes. To prepare the RT reaction mix on ice (per 20-μL reaction): (1) Allow the kit components to thaw on ice and (2) Calculate the volume of components needed to prepare the required number of reactions as shown in Table 2:

TABLE 2

| RT reaction mix | Volume/Reaction |
|---|---|
| 2X RT Buffer | 10 μl |
| 20X RT Buffer | 1 μl |
| Nuclease-free water | Q.S.P. 20 μl |
| Sample (0.5 or 1 μg RNA) | Up to 9 μl |

Distribute 20 μl of RT reaction mix into tubes. Seal the tubes and centrifuge them at 230×g for 1 min. To perform the RT reaction, program the thermal cycler as follows: 37° C.→60 minutes; 95° C.→5 minutes; 12° C.→∞.

8.3.5. Quantitative Gene Expression Assays (Real-Time PCR))

Step 1—Prepare the cDNA Sample:

Isolate total RNA using the Ribopure Ambion® RNA isolation kit (Applied Biosystems AM 1924) and determine the RNA concentration by Nanodrop spectrophotometer with 1.5 μl of sample (see Section 8.3.2). Perform reverse transcription (RT) using the High Capacity RNA-to cDNA Kit (Applied Biosystems PN 4387406) (see Section 8.3.4). Store the cDNA samples at −20° C., if you do not proceed immediately to PCR.

Step 2—Prepare the PCR Reaction Mix:

Use the same amount of cDNA for all samples (4 μl of 20 μL RT reaction on 0.5 or 1 μg RNA). For each sample (to be run in triplicate), sample the following into a nuclease-free 1.5-mL microcentrifuge tube (add the volume for 2 samples more to calculate the final volume of PCR reaction mix): 2× TaqMan® Gene Expression Master Mix: 10 μl; 20× TaqMan® Gene Expression Assay: 1 μl; Nuclease free H₂O: 5 μl. Cap the tube and invert it several times to mix the reaction components. Centrifuge the tube briefly.

Step 3—Load the Plate:

Put 4 μl of cDNA into each well of a 96-well reaction plate and transfer 16 μL of PCR reaction mix per well by changing the direction of the plate for the two deposits (Foresee one well with 4 μl of H₂O to make blank for each gene). Seal the plate with the appropriate cover. Centrifuge the plate briefly (230×g for 1 min). Load the plate into StepOnePlus Real Time PCR system.

Step 4—Run the Plate:

Create an experiment/plate document for the run. Run the plate. Program: (a) 95° C.→10 min; (b) 95° C.→15 sec; (c) 60° C.→1 min; with 40 cycles of (b) and (c).

8.3.6. Radioactive Cholesterol Efflux Study

Day 1—Cell culture: J774 macrophages obtained from ATCC (N^{o} TIB-67) were grown in Dulbecco's modified Eagle's medium (DMEM, Invitrogen) supplemented with 10% FBS (foetal bovine serum, Invitrogen), 100 units/ml penicillin G (Invitrogen), and 100 units/ml streptomycin (Invitrogen) at 37° C. with 5% CO₂. Cells were seeded on 24-well plates (Falcon) at 40,000 cells/well and grown for 32 hours in 2 ml DMEM 10% FBS. LDL oxidation: 1 ml LDL is dialyzed against 4 L PBS (twice, 12 hours each) in Slide-A-Lyzer™ Mini Dialysis Units 7000MWCO (Pierce).

Day 2—LDL Oxidation:

[1] After dialysis, proteins-LDL are quantified with Coomassie protein assay (#1856209, ThermoScientific) using albumin (#23209, ThermoScientific) as standards. Absorbance is read with Glomax multi detection System (Promega) at 600 nm. PBS-dialysed LDL (2 mg/ml) were oxidized using CuSO₄ (5 μM final concentration) (C8027, Sigma Aldrich) for 4 hours at 37° C. The reaction was stopped by adding EDTA (100 μM final concentration) (#20302.236, Prolablo). The oxidized LDL were dialysed against 2×1 L PBS for 0.5 hours. After dialysis, proteins-LDL are quantified by same method as [1]. Cell culture: Oxidised LDL (50 μl, 12.5 μg) are mixed with [³H] cholesterol (1 μCi, Perkin Elmer) in DMEM 2.5% FBS for 15 minutes. Radiolabelled LDL are added to J744 cells in 450 μl DMEM 2.5% FBS for 24 hours.

Day 3—Cell Culture:

Radioactive medium is removed and cells are washed three times with 1 ml DMEM (without FBS) and incubated with or without agonist LXR (1 μM) overnight.

Day 4—Cholesterol Efflux Assay:

The efflux is induced by adding different acceptors for 6 hours (or different time between 1 to 24 hours) in 250 μl DMEM without FBS. Radioactivity was measured by adding the medium (0.25 ml) to Super Mix (0.75 ml) (1200-439 Perkin-Elmer), mixed in 24 well flexible microplate (1450-402 Perkin-Elmer) and radioactivity was measured with MicroBeta® Trilux (2 minutes counting time). The intracellular [³H] cholesterol was extracted by 0.2 ml hexane-isopropanol (3:2) (incubation 0.5 hours) and measured by liquid scintillation counting.

8.3.7. Membrane/Cytosol Separation

Membrane/Cytosol separation without ultracentrifugation: Resuspend cell pellet (2 wells of 6-well plate) in 200 μl lysis buffer or tissue sample (50-100 mg) in 1 ml lysis buffer. Homogenize tissue sample with Turrax® or cell pellet by sonication 2×10 s at 30% of amplitude using the Digital Sonifier® BRANSON. Centrifuge at 800×g for 5 min at 4° C. Transfer the supernatant in a new tube and centrifuge 30 min at 13,000×g at 4° C., save the supernatant (cytosol fraction). Resuspend the pellet in 100-200 μl lysis buffer (supplemented with 1.2% Triton X100). Put under strong agitation during 15 min. Centrifuge 5 min at 14,000×g, save the supernatant (solubilized membrane protein fraction).

Membrane/Cytosol Separation with Ultracentrifugation:

Resuspend in 1 ml lysis buffer a cell pellet (2 wells of 6-well plate) or a tissue sample (50-100 mg). Homogenize tissue sample with Turrax® or cell pellet with sonification 2×10 s at 30% of amplitude using the Digital Sonifier® BRANSON. Centrifuge at 800×g for 5 min at 4° C. Transfer the supernatant in a tube for ultracentrifugation and centrifuge 1 hour at 100,000×g (38,500 rpm) at 8° C. (rotor Ti70), save the supernatant (cytosol fraction). Resuspend the pellet in 100-200 μl lysis buffer (Table 3) (supplemented with 1.2% Triton X100). Put under strong agitation during 15 min. Centrifuge 5 min at 14,000×g, save the supernatant (solubilized membrane protein fraction).

TABLE 3

| Components of Lysis Buffer | For 10 ml Buffer |
|---|---|
| 20 mM Tris | 200 μl Tris 1M pH 7.5 |
| 150 mM NaCl | 375 μl NaCl 4M |
| 1 mM EDTA | 20 μl EDTA 0.5M |
| 2 mM MgCl2 | 20 μl MgCl2 1M |
| 1X protease inhibitor | 100 μl IP 100X |
| | 9285 μl H₂O |

8.4. Results of Gene Regulation Studies A-P

8.4.1. Study A: J774 ABCA1 Gene Regulation by CER-001, HDL₃ and ApoA-I—Dose Response (25, 250 and 1000 μg/mL)

In this study, the ABCA1 gene expression in mouse macrophages (J774) in the conditions of cholesterol efflux for different concentrations of CER-001, HDL₃ and ApoA-I was examined. J774 were seeded on 6× well plates (300,000 cells/well) and loaded with oxidized-LDL without the use of ³H-cholesterol. CER-001, HDL₃ (from a frozen stock solution) and ApoA-I (25, 250 and 1000 μg/mL) were added for 6 hours on the macrophages and the RNA were extracted with the RiboPure™ kit according to the manufacturer's protocol (one well per condition). Gene expression was assayed using the protocols described in Sections 8.3.2 (RNA extraction); 8.3.4 (reverse transcription), and 8.3.5 (qPCR). ABCA1 gene expression was determined with Taqman probe Mm00442646.m1 according to the manufacturer's protocol. The reference gene used is HPRT1 (Taqman probe: Mm00446968.m1).

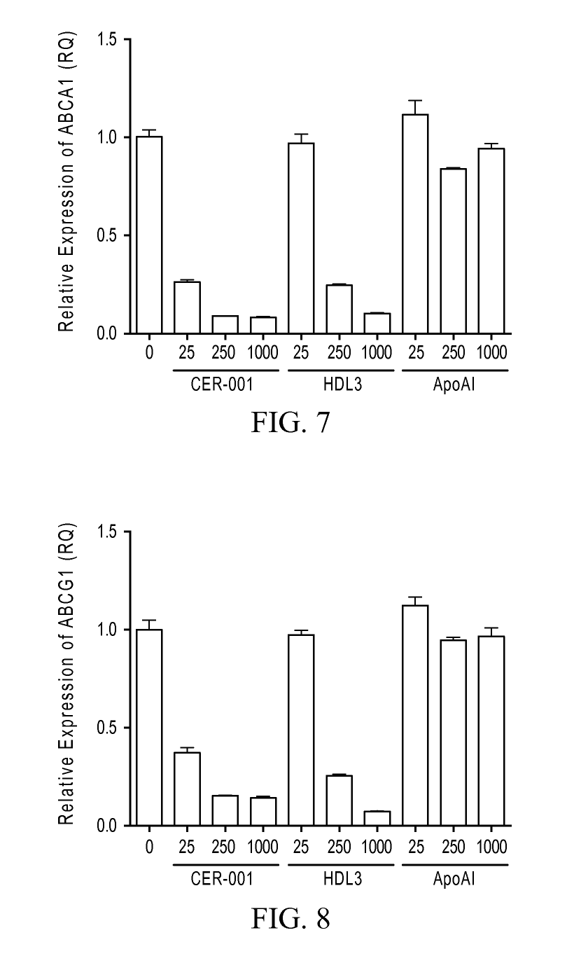

After a 6 hour incubation, ApoA-I did not change the ABCA1 expression for the doses used in the experiment. CER-001 decreased the ABCA1 mRNA at all the doses; HDL₃ did not affect ABCA1 mRNA concentration at 25 μg/mL dose (FIG. 7).

8.4.2. Study B: J774 ABCG1 Gene Regulation by CER-001, HDL₃ and ApoA-I—Dose Response (25, 250 and 1000 μg/mL)

In this study, the ABCG1 gene expression in mouse macrophages (J774) in the conditions of cholesterol efflux for different concentrations of CER-001, HDL₃ and ApoA-I was examined. J774 were seeded on 6 well plates (300,000 cells/well) and loaded with oxidized-LDL without the use of ³H-cholesterol. CER-001, HDL₃ (from a frozen stock solution) and ApoA-I (25, 250 and 1000 μg/mL) were added for 6 hours on the macrophages and the RNA were extracted with the RiboPure™ kit according to the manufacturer's protocol (one well per condition). Gene expression was assayed using the protocols described in Sections 8.3.2 (RNA extraction); 8.3.4 (reverse transcription), and 8.3.5 (qPCR). ABCG1 gene expression was determined with Taqman probe Mm00437390.m1 according to the manufacturer's protocol. The reference gene used is HPRT1 (Taqman probe: Mm00446968.m1).

ApoA-I did not change the ABCG1 expression for the doses used in the experiment. CER-001 decreased the ABCG1 mRNA at all the doses; HDL₃ did not affect ABCG1 mRNA concentration at 25 μg/mL dose (FIG. 8).

8.4.3. Study C: J774 SR-BI Gene Regulation by CER-001, HDL₃ and ApoA-I-—Dose Response (25, 250 and 1000 μg/mL)

In this study, the SR-BI gene expression in mouse macrophages (J774) in the conditions of cholesterol efflux for different concentrations of CER-001, HDL₃ and ApoA-I was examined. J774 were seeded on 6 well plates (300,000 cells/well) and loaded with oxidized-LDL without the use of 3H-cholesterol. CER-001, HDL₃ (from a frozen stock stolution) and ApoA-I (25, 250 and 1000 μg/mL) were added for 6 hours on the macrophages and the RNA were extracted with the RiboPure™ kit according to the manufacturer's protocol (one well per condition). Gene expression was assayed using the protocols described in Sections 8.3.2 (RNA extraction); 8.3.4 (reverse transcription), and 8.3.5 (qPCR). SR-BI gene expression was determined with Taqman probe Mm00450234.m1 according to the manufacturer's protocol. The reference gene used is HPRT1 (Taqman probe: Mm00446968.m1).

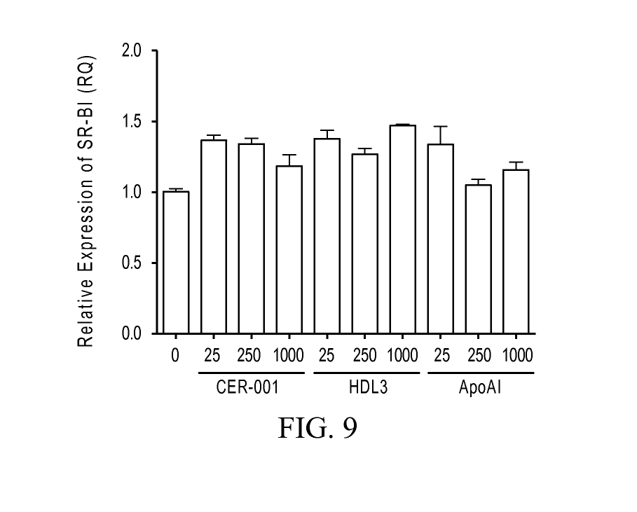

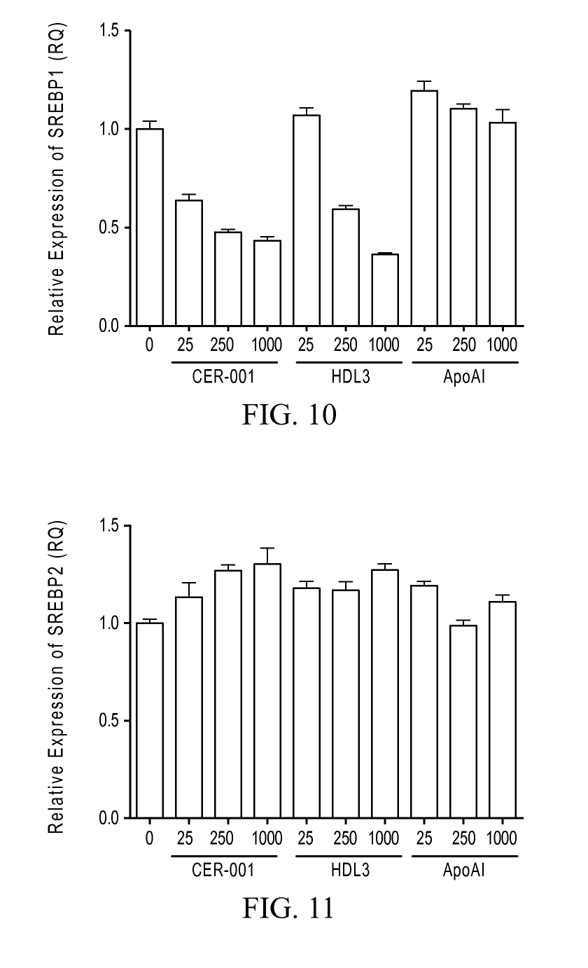

No significant changes in SR-BI gene expression were observed for the different treatments at all the doses (FIG. 9).

8.4.4. Study D: J774 Other Gene Regulations by CER-001, HDL₃ and ApoA-I—Dose Response (25, 250 and 1000 μg/mL)

The mRNA regulation of those genes expressing ABCA1, ABCG1 and SR-BI is linked to nuclear proteins as LXR, SREBP1 and SREBP2. This study examines the mRNA levels of LXR, SREBP1 and SREBP2 in mouse macrophages (J774) in the conditions of cholesterol efflux for different concentrations of CER-001, HDL₃ and ApoA-I. J774 were seeded on 6 well plates (300,000 cells/well) and loaded with oxidized-LDL without the use of 3H-cholesterol. CER-001, HDL₃ and ApoA-I (25, 250 and 1000 μg/mL) were added for 6 hours on the macrophages and the RNA were extracted with the RiboPure™ kit according to the manufacturer's protocol (one well per condition). Gene expression was assayed using the protocols described in Sections 8.3.2 (RNA extraction); 8.3.4 (reverse transcription), and 8.3.5 (qPCR). SREBP-1, SREBP-2 and LXR gene expression levels were determined with Taqman probe (Mm01138344.m1, Mm01306292.m1, Mm00443451.m1 respectively) according to the manufacturer's protocol. The reference gene used is HPRT1 (Taqman probe: Mm00446968.m1).

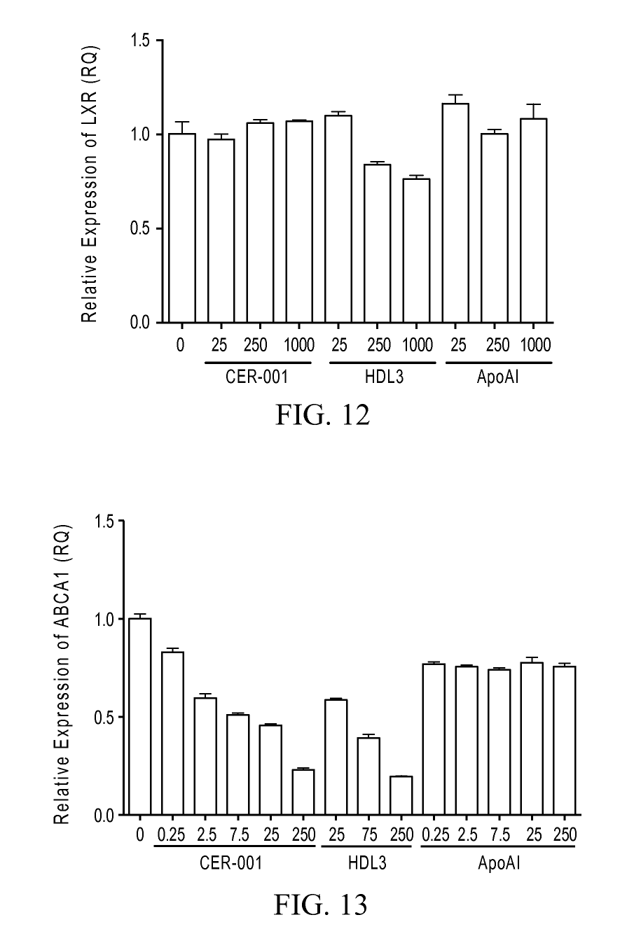

No significant changes in SREBP-1, SREBP-2 and LXR gene expression were observed for the different treatments with ApoA-I. CER-001 and HDL₃ only affected SREBP-1 mRNA levels (FIG. 10) for the different doses while SREBP-2 and LXR were not changed (FIG. 11 and FIG. 12, respectively).

8.4.5. Study E: CER-001 and HDL₃ EC₅₀ Determination for the Regulation of ABCA1, ABCG1 and SR-BI Expression in J774 Mouse Macrophages

This study examined the minimum effective concentration of ApoA-I, CER-001 or HDL₃ needed for the regulation of ABCA1, ABCG1 and SR-BI gene expression. J774 were seeded on 6 well plates (300,000 cells/well) and loaded with oxidized-LDL. CER-001, HDL₃ and ApoA-I (0.25, 2.5, 7.5, 25 and 250 μg/mL) were added for 6 hours on the macrophages and the RNA were extracted with the RiboPure™ kit according to the manufacturer's protocol. Gene expression was assayed using the protocols described in Sections 8.3.2 (RNA extraction); 8.3.4 (reverse transcription), and 8.3.5 (qPCR). SR-BI gene (Taqman probe Mm00450234.m1), ABCG1 (Taqman probe Mm00437390.m1), SREBP1 (Taqman probe Mm01138344.m1) and ABCA1 (Taqman probe Mm00442646.m1) expression were determined according to the manufacturer's protocol. The reference gene used is HPRT1 (Taqman probe: Mm00446968.m1).

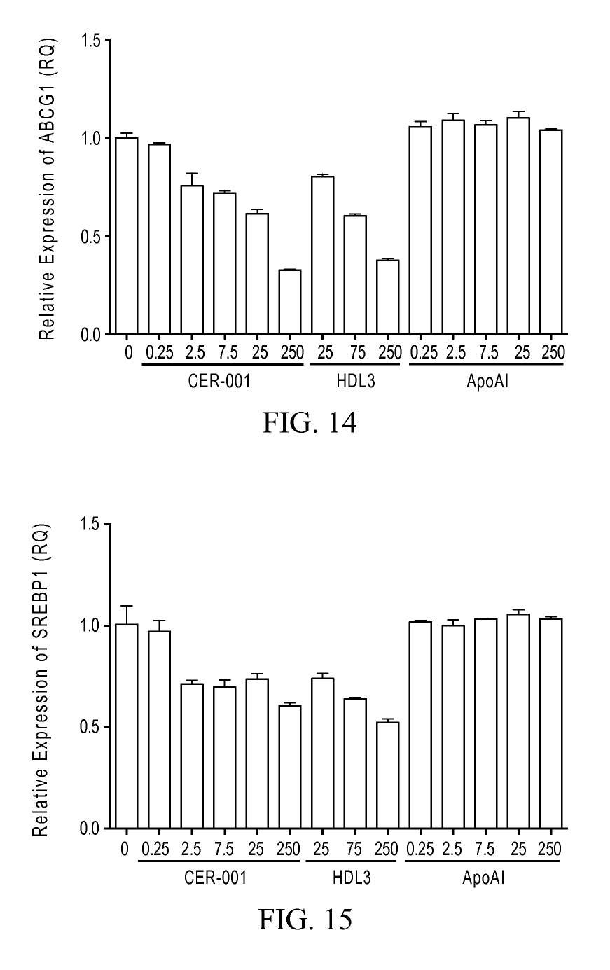

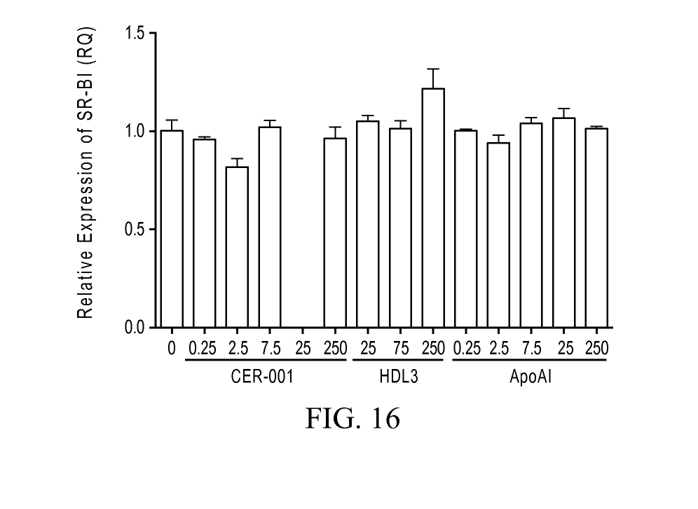

ApoA-I did not change the mRNA level of the genes tested (FIG. 13). The CER-001 dose for diminishing half of the ABCA1 level is around 7.5 μg/mL, and 25 μg/mL for HDL₃ (FIG. 13). For ABCG1, doses above 75 μg/mL for CER-001 and HDL₃ are necessary to decrease half of the mRNA level (FIG. 14). For SREBP1, we observed a decrease and a plateau for concentrations above 2.5 μg/mL for CER-001 and 25 μg/mL for HDL₃ (FIG. 15). SR-BI level was not affected by the different treatments (FIG. 16).

8.4.6. Study F: Kinetics for the Regulation of ABCA1 mRNA by CER-001 and HDL₃ in J774 Mouse Macrophages

This study examined the kinetics of decreasing the mRNA level of ABCA1 in J774 macrophages. J774 were seeded on 6 well plates (300,000 cells/well) and loaded with oxidized-LDL. CER-001, HDL₃ and ApoA-I (25 and 250 μg/mL) were added for different time points on the macrophages and the RNA were extracted with the RiboPure™ kit according to the manufacturer's protocol. Gene expression was assayed using the protocols described in Sections 8.3.2 (RNA extraction); 8.3.4 (reverse transcription), and 8.3.5 (qPCR). ABCA1 (Taqman probe Mm00442646.m1) expression was determined according to the manufacturer's protocol. The reference gene used is HPRT1 (Taqman probe: Mm00446968.m1).

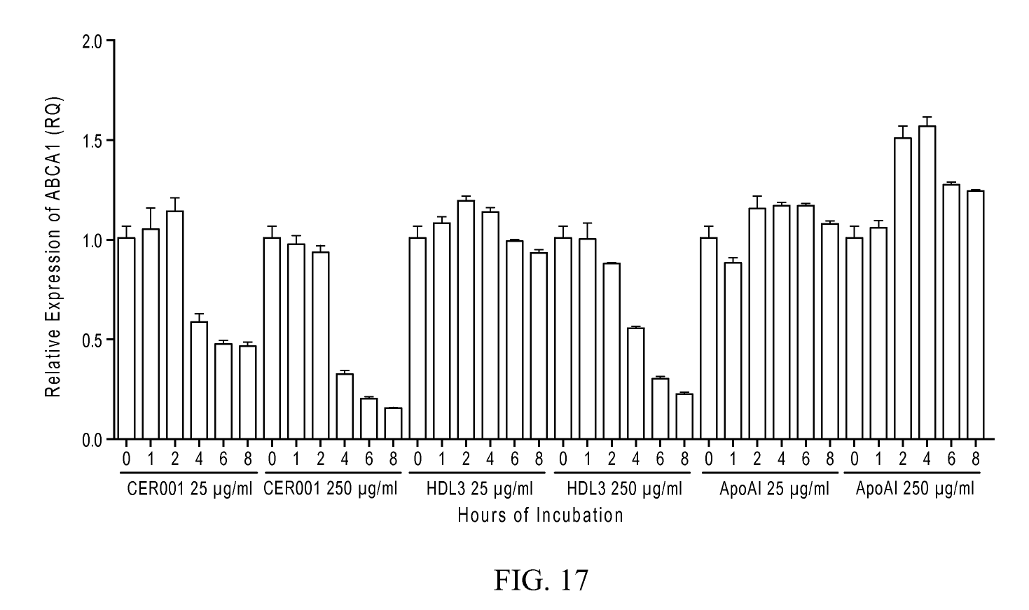

CER-001 (25 or 250 μg/mL) was able to decrease half of the ABCA1 mRNA level in 4 hours. The behavior of HDL₃ (250 μg/mL) (which has been thawed/frozen several times) is similar to CER-001, except no down-regulation was observed at 25 μg/mL HDL₃. As previously reported, ApoA-I did not decrease the mRNA ABCA1 level for either concentrations 25 μg/mL or 250 μg/mL. An increase of ABCA1 mRNA was observed at 2 and 4 hours with ApoA-I treatment (FIG. 17).

8.4.7. Study G: Camp Effect on the Regulation of ABCA1 and ABCG1 mRNA Levels in the Presence of CER-001, HDL₃ and ApoA-I

This study examined the effect of CER-001, HDL₃ or ApoA-I on ABCA1 and ABCG1 mRNA level in J774 macrophages after treatment with cAMP. J774 were seeded on 6 well plates (300,000 cells/well) and loaded with oxidized-LDL. The next day, medium was replaced by DMEM+/−cAMP (300 μM). After overnight incubation in presence/absence of cAMP, the medium was removed and replaced with DMEM mixed with CER-001, or HDL₃ or ApoA-I (250 μg/mL) for 6 hours and the RNA were extracted with the RiboPure™ kit according to the manufacturer's protocol. Gene expression was assayed using the protocols described in Sections 8.3.2 (RNA extraction); 8.3.4 (reverse transcription), and 8.3.5 (qPCR). ABCG1 (Taqman probe Mm00437390.m1) and ABCA1 (Taqman probe Mm00442646.m1) expression were determined according to the manufacturer's protocol. The reference gene used is HPRT1 (Taqman probe: Mm00446968.m1).

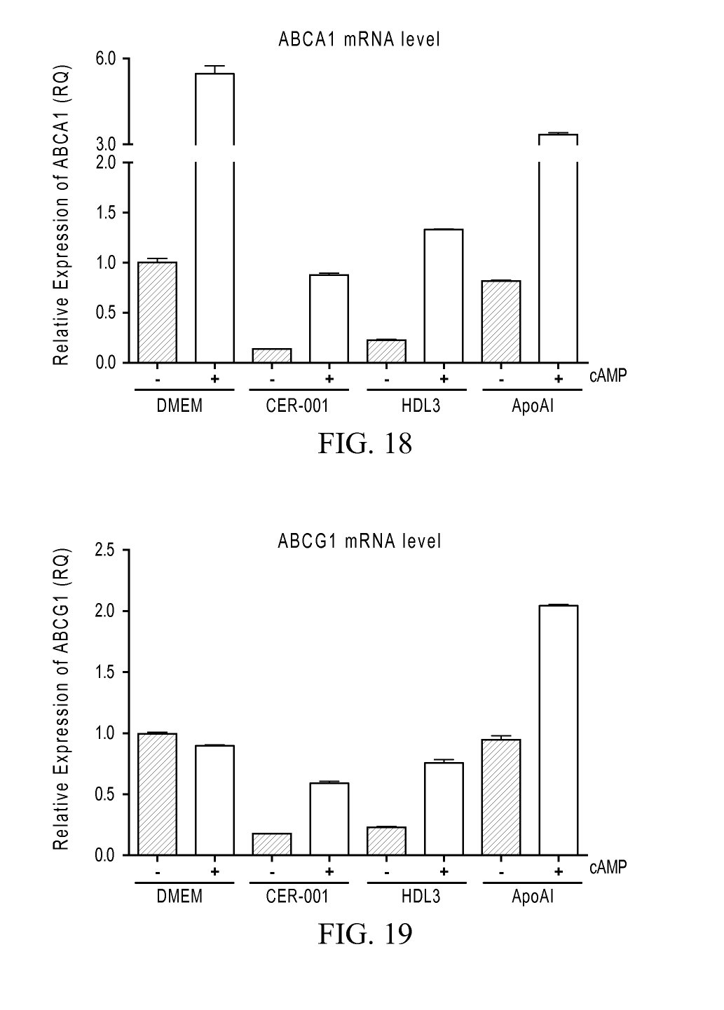

In the presence of cAMP, an increase in the ABCA1 mRNA level was observed (FIG. 18). In the presence of CER-001 or HDL₃ (250 μg/mL), the mRNA levels of ABCA1 and ABCG1 was decreased while ApoA-I was not altered. In the presence of cAMP, the concentrations of ABCA1 and ABCG1 when cells were incubated with CER-001 or HDL₃ were back to RQ=1 but the stimulation of ABCA1 (RQ=5-6) was not reached (FIG. 18 and FIG. 19) In the presence of ApoA-I and cAMP, the mRNA levels of ABCA1 (RQ≈3) was increased compared to ApoA-I alone but not to the same level as for DMEM+cAMP (RQ≈6).

8.4.8. Study H: Effect on the Regulation of ABCA1 Protein Level in J774 Macrophages in the Presence of CER-001 and HDL₃

This study examined the effect of CER-001 and HDL₃ on the protein level of ABCA1 in J774 macrophages. J774 macrophages were seeded on 6 well plates (300,000 cells/well) and loaded with oxidized-LDL. The next day, medium was replaced by DMEM. After overnight equilibration, the medium was removed and replaced with DMEM mixed with CER-001 and HDL₃ (250 μg/mL) for 6 hours and the cells were lysed and membranes separated according to the method of Section 8.3.7. Cytosolic and membrane proteins were resolved on SDS PAGE 10% and probed against ABCA1 (ab7360—dilution 1/1000). Protein level was quantified using imageJ® software.

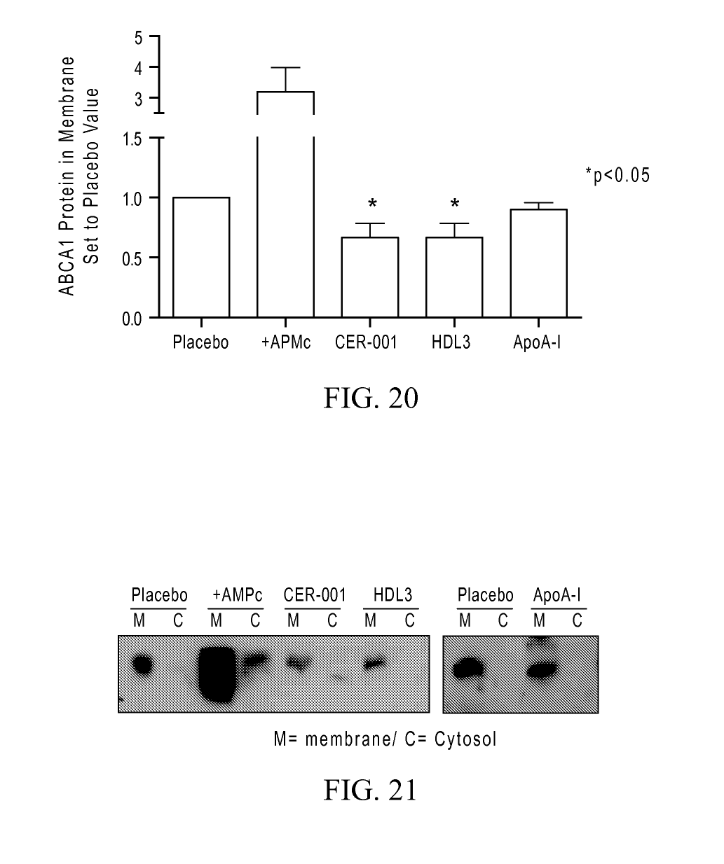

A significant decrease of ABCA1 protein level for macrophages treated with CER-001 and HDL₃ was observed (FIG. 20 and FIG. 21). ApoA-I did not affect the level of ABCA1 and the addition of cAMP strongly increased this level. Addition of CER-001 and HDL₃ at 250 μg/mL for 6 h on J774 macrophages reduced the mRNA and protein levels of ABCA1.

8.4.9. Study I: cAMP Effect on the Regulation of ABCA1 and ABCG1 mRNA Level in the Presence of Increasing Concentrations of CER-001

This study examines the effect of increasing concentrations of CER-001 on the mRNA level of ABCA1 and ABCG1 after treatment with cAMP. J774 were seeded on 6 well plates (300,000 cells/well) and loaded with oxidized-LDL. The next day, medium was replaced by DMEM+/−cAMP (300 μM). After overnight incubation in presence/absence of cAMP, the medium was removed and replaced with DMEM mixed with CER-001, at different concentrations (0, 0.1, 0.5, 1, 2, 4, 6, 8, 10, 15 and 30 μg/mL) for 6 hours and the RNA were extracted with the RiboPure™ kit according to the manufacturer's protocol. Gene expression was assayed using the protocols described in Sections 8.3.2 (RNA extraction); 8.3.4 (reverse transcription), and 8.3.5 (qPCR). ABCG1 (Taqman probe Mm00437390.m1) and ABCA1 (Taqman probe Mm00442646.m1) expression were determined according to the manufacturer's protocol. The reference gene used is HPRT1 (Taqman probe: Mm00446968.m1).

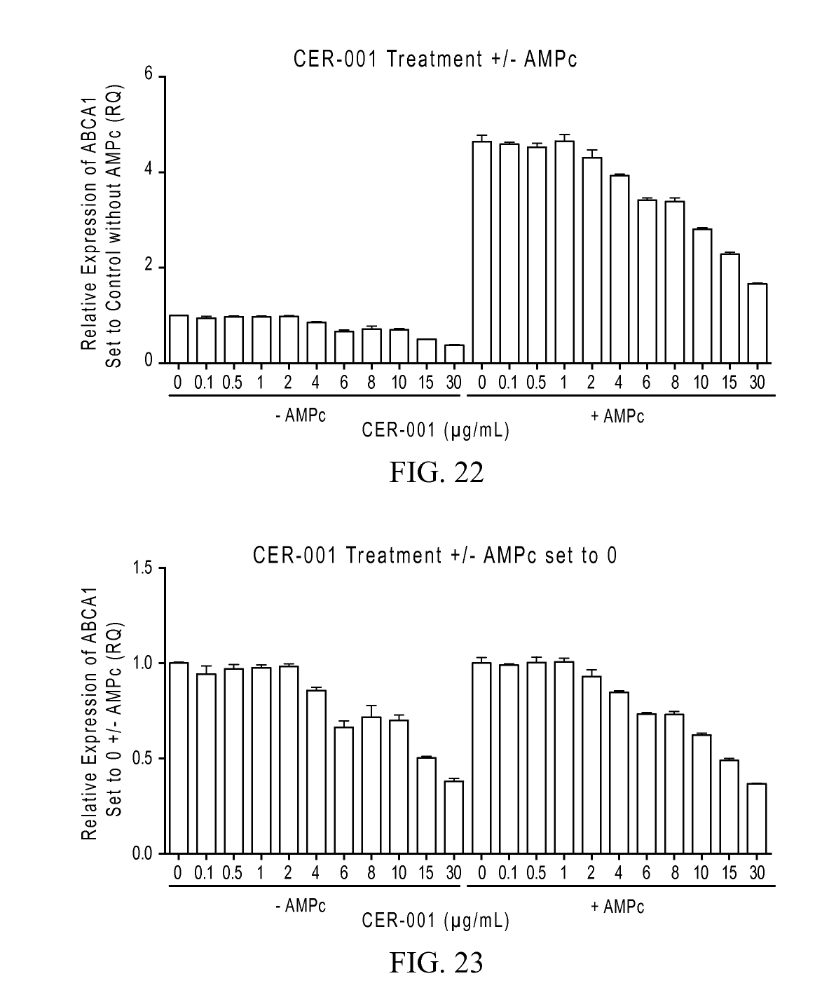

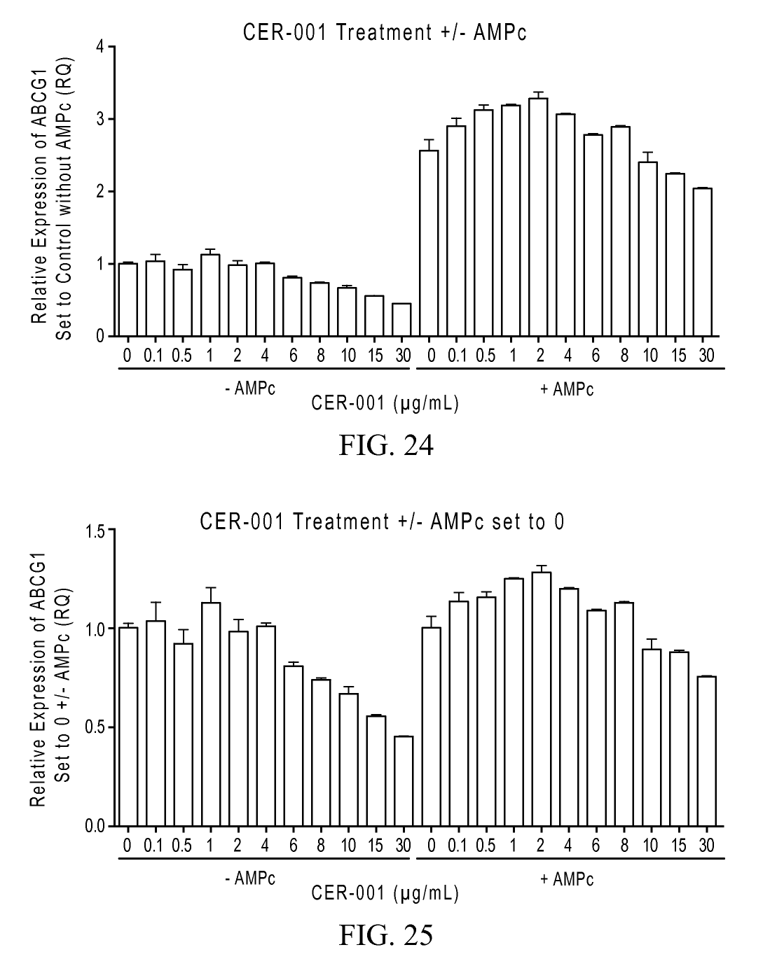

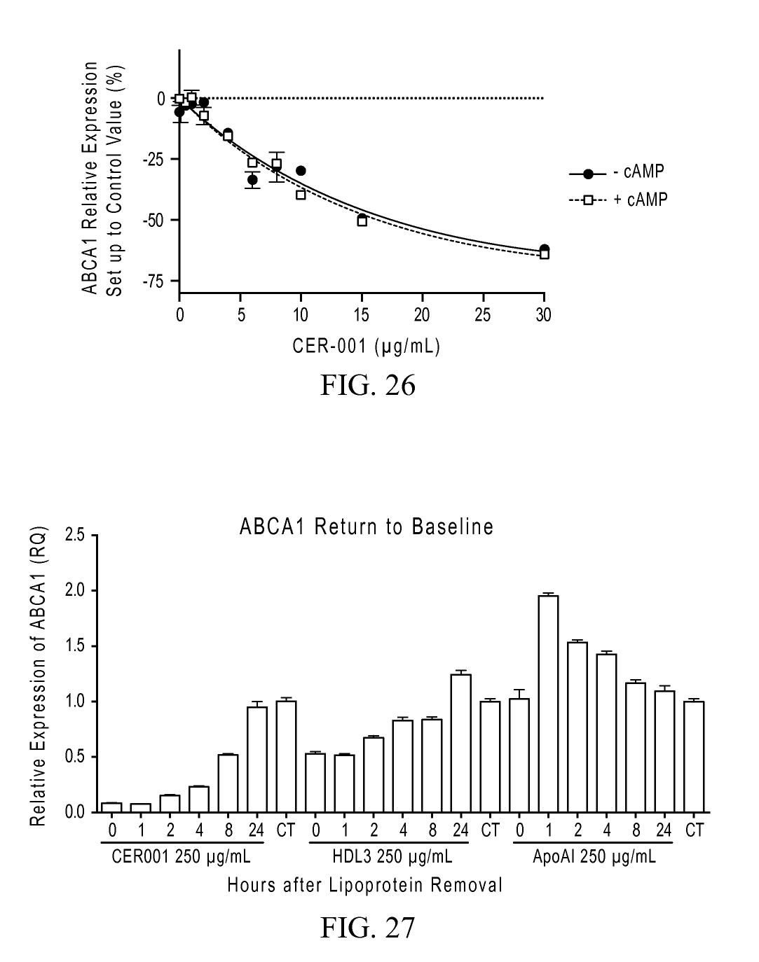

In the presence of cAMP an increase for ABCA1 and ABCG1 mRNA level was observed (FIG. 22, FIG. 23, FIG. 24, FIG. 25, and FIG. 26). The decrease of ABCA1 and ABCG1 mRNA level was observed at 4-6 μg/mL doses with a maximum around 15 μg/mL. The cAMP activation did not change the sufficient dose of CER-001 for decreasing the level of ABCA1 because the profiles in presence or absence of cAMP were similar (FIG. 26).

8.4.10. Study J: Return to Normal Amount of ABCA1 and ABCG1 mRNA after Treatment with CER-001 and HDL₃

This study examined the time necessary to return to the normal amount of ABCA1 and ABCG1 mRNA after treatment with CER-001 and HDL₃. J774 were seeded on 6 well plates (600,000 cells/well) in DMEM 10% FCS. The next day, medium was replaced by DMEM without serum and treated 24 hours with CER-001, HDL₃ or ApoA-I (250 μg/mL). Medium was removed and the macrophages were washed with DMEM. At different time points (0, 1, 2, 4, 8 and 24 hours) post CER-001, HDL₃ or ApoA-I removal, cellular RNA was extracted with the RiboPure™ kit according to the manufacturer's protocol. Gene expression was assayed using the protocols described in Sections 8.3.2 (RNA extraction); 8.3.4 (reverse transcription), and 8.3.5 (qPCR). ABCG1 (Taqman probe Mm00437390.m1), ABCA1 (Taqman probe Mm00442646.m1) and SR-BI (Taqman probe Mm00450234.m1) expression were determined according to the manufacturer's protocol. The reference gene used is HPRT1 (Taqman probe: Mm00446968.m1).

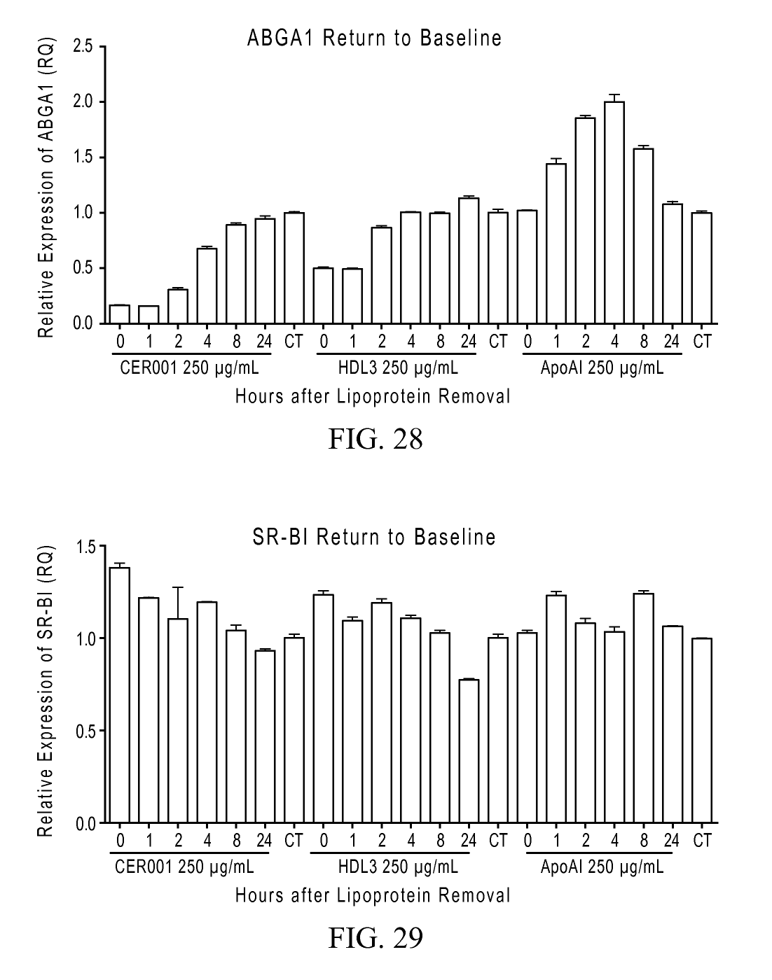

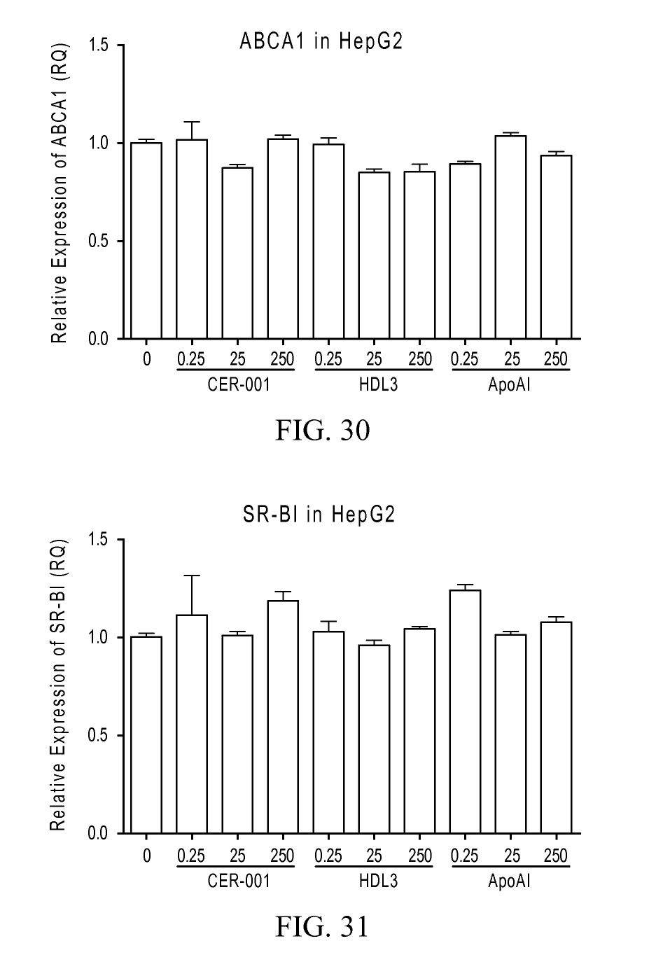

A decrease for ABCA1 and ABCG1 mRNA levels was observed after CER-001 and HDL₃ treatment. ApoA-I did not affect the levels of those mRNA and CER-001 and HDL₃ do not change the mRNA level of SR-BI (FIG. 27, FIG. 28, and FIG. 29). After CER-001 treatment, the mRNA level of ABCA1 returned to baseline in more than 8 hours and for ABCG1, the return was faster because baseline was reached in 8 hours. After HDL₃ treatment, the mRNA level of ABCA1 returned to baseline in approximately 8 hours and for ABCG1, 2 to 4 hours were necessary. The difference observed between CER-001 and HDL₃ treatments is probably due to a lower level of mRNA in the presence of CER-001. Removal of ApoA-I induced an increase in ABCA1 and ABCG1 mRNA levels at different time points (1 hour for ABCA1 and 4 hours for ABCG1). CT stands for control, i.e., J774 macrophages grown without addition of CER-001, HDL₃ or ApoA-I.

8.4.11. Study K: Macrophage Specificity for the Regulation of ABCA1 and SR-BI mRNA by CER-001 and HDL₃—Effect on Hepatocytes (Mouse and Human)

This study examined the effect of CER-001 and HDL₃ (at 25 μg/mL) on ABCA1 and SR-BI mRNA levels in mouse and human hepatocytes. HepG2 (human hepatocytes) and Hepa1-6 (mouse hepatocytes) were seeded on 6 well plates (300,000 cells/well) in DMEM 10% FCS. Three days later CER-001, HDL₃ and ApoA-I (0.25, 25 and 250 μg/mL in DMEM) were added for 6 hours on the hepatocytes and the RNA were extracted with the RiboPure™ kit according to the manufacturer's protocol. Gene expression was assayed using the protocols described in Sections 8.3.2 (RNA extraction); 8.3.4 (reverse transcription), and 8.3.5 (qPCR). ABCA1 (Taqman probe Hs01059118.m1 and Mm00442646.m1 for respectively HepG2 and Hepa1-6) and SR-BI (Taqman probe Hs00969821.m1 and Mm00450234.m1 for respectively HepG2 and Hepa1-6) expression was determined according to the manufacturer's protocol. The reference gene used is HPRT1 (Taqman probe: Mm00446968.m1) for Hepa1-6 and GAPDH (Taqman probe: Hs03929097.g1) for HepG2 cells.

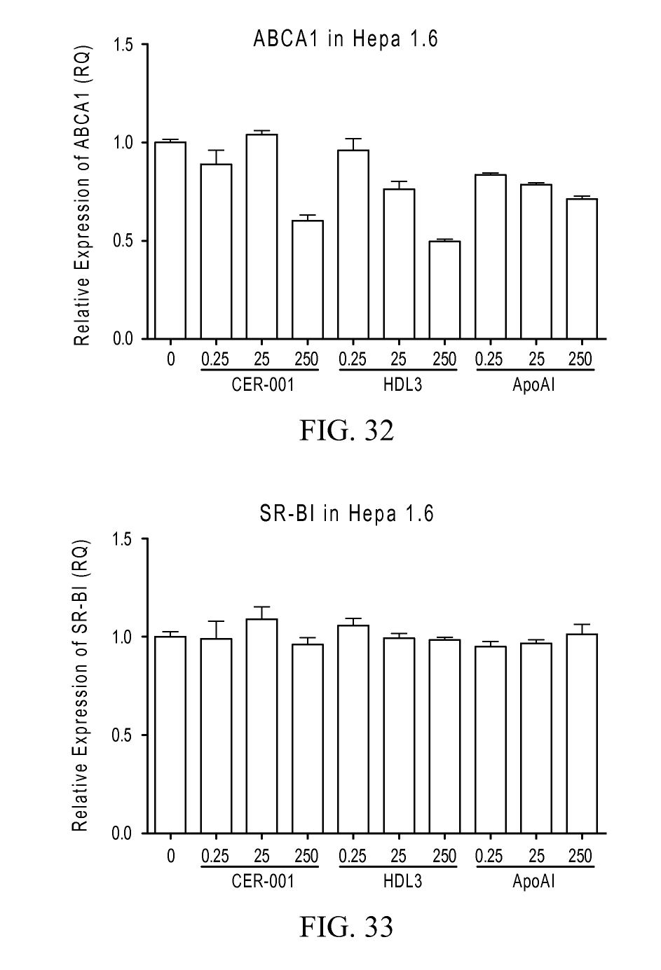

No significant decrease of ABCA1 and SR-BI mRNA levels was observed in human hepatocytes for CER-001, HDL₃ and ApoA-I treatments (FIG. 30 and FIG. 31). There was a two-fold decrease observed in ABCA1 mRNA levels in mouse hepatocytes for CER-001 and HDL₃ treatments at 250 μg/mL (FIG. 32 and FIG. 33). Treatment at 250 μg/mL with ApoA-I decreased by 25% the ABCA1 mRNA level in mouse hepatocytes.

8.4.12. Study L: Consequence of ApoA-I Addition after ABCA1 Down-Regulation by CER-001 and HDL₃

This study examined the effect of ApoA-I addition on gene expression after down-regulation of ABCA1 and ABCG1 by CER-001 and HDL₃. J774 were seeded on 6 well plates (300,000 cells/well) in DMEM 2.5% FCS. After equilibration (DMEM), CER-001, HDL₃ and ApoA-I are added overnight at 250 μg/mL. The next day, medium was replaced by fresh DMEM supplemented with or without ApoA-I (25 or 250 μg/mL) to initiate ApoA-I cholesterol efflux for 2 hours. The experiment was stopped at different time points: 1) J774 stopped before addition of ApoA-I, 2) J774+DMEM (passive efflux for 2 hours), 3) J774+ApoA-I (25 μg/mL) for 2 hours and 4) J774+ApoA-I (250 μg/mL) for 2 hours. The RNA was extracted with the RiboPure™ kit according to the manufacturer's protocol. Gene expression was assayed using the protocols described in Sections 8.3.2 (RNA extraction); 8.3.4 (reverse transcription), and 8.3.5 (qPCR). ABCA1 (Taqman probe Mm00442646.m1), ABCG1 (Taqman probe Mm00437390.m1), and SR-BI (Taqman probe Mm00450234.m1) expression were determined according to the manufacturer's protocol. The reference gene used is HPRT1 (Taqman probe: Mm00446968.m1).

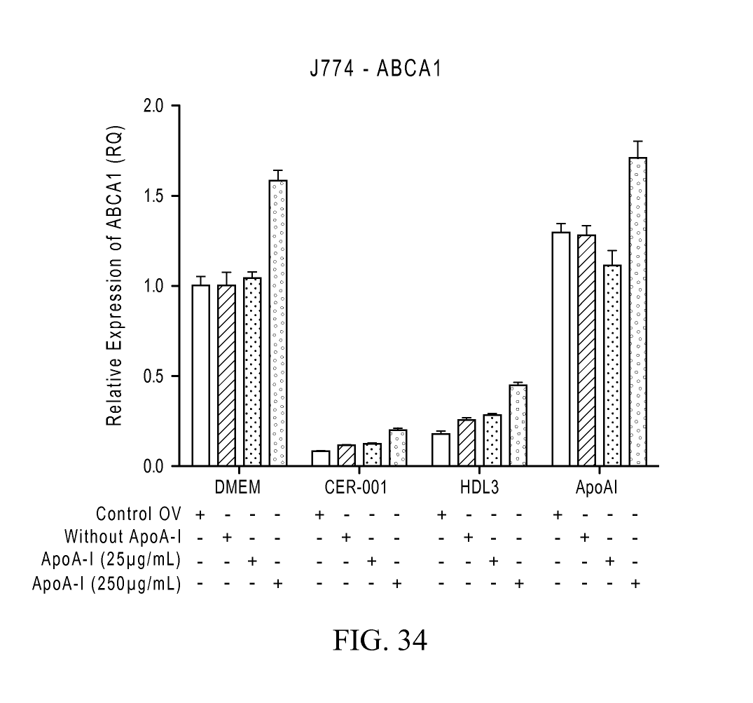

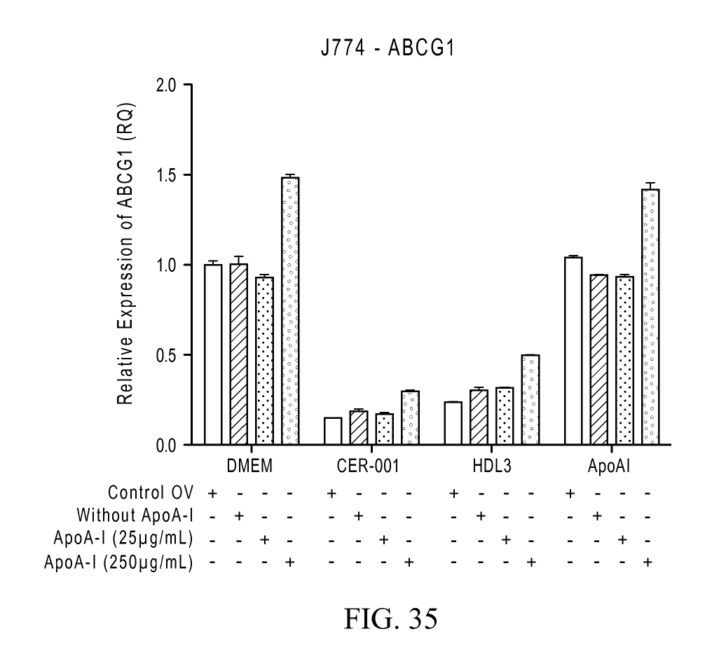

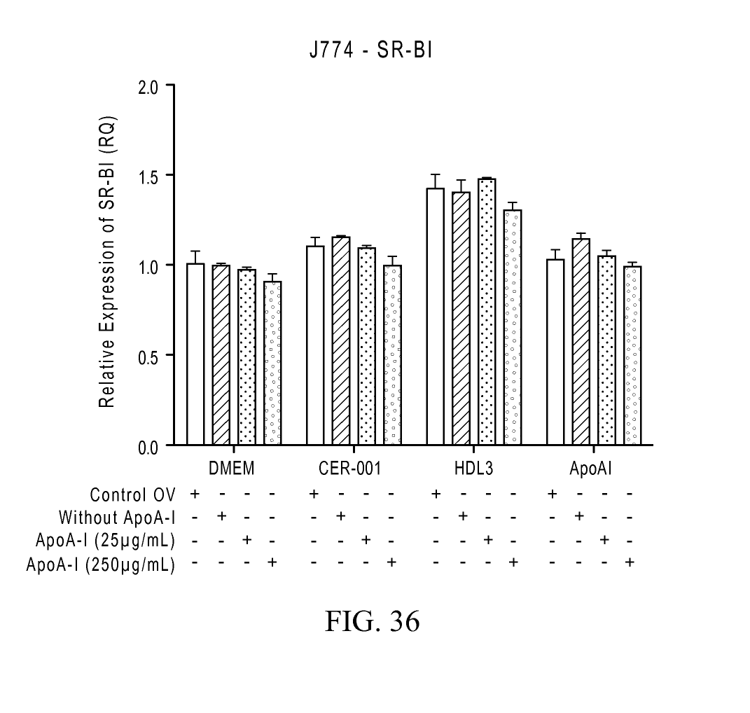

The addition of ApoA-I at 250 μg/mL increased the ABCA1 mRNA level after 2 hours (DMEM condition—4^{th} bar) (FIG. 34). This increase was transitory because in 6 hours the level was back to baseline (see Expt. F). The addition of CER-001 or HDL₃ strongly decreased the ABCA1 mRNA level (black bars). Two hours after removing the lipoproteins, the ABCA1 mRNA level was increasing accordingly to previous results (see Expt. I) and this increase was boosted by the addition of ApoA-I at 250 μg/mL. The pre-incubation of macrophages with ApoA-I 250 μg/mL did not change the ABCA1 mRNA level. The stimulation noted with DMEM+ApoA-I at 250 μg/mL was also observed in those conditions with ApoA-I pre-incubation at 250 μg/mL. A similar profile was observed for ABCG1 mRNA regulation for the different conditions (FIG. 35). SR-BI mRNA increased in the presence of HDL₃ but not for the other conditions (FIG. 36). The addition of ApoA-I did not change the SR-BI mRNA level for the different conditions tested.

8.4.13. Study M: Regulation of ABCA1, ABCG1 and SR-BI mRNA Cellular Level by HDL₂ in J774 Macrophages

This study examined the effect of HDL₂ on ABCA1, ABCG1 and SR-BI mRNA levels in mouse macrophages. HDL₂ is a bigger and more mature lipoprotein compared to HDL₃ and HDL₂ interacts with ABCG1 and HDL₃ with ABCA1. J774 were seeded on 6 well plates (300,000 cells/well) and loaded with oxidized-LDL. HDL₂ (from 2.5 to 1000 μg/mL) were added for 6 hours on the macrophages and the RNA were extracted with the RiboPure™ kit according to the manufacturer's protocol. Gene expression was assayed using the protocols described in Sections 8.3.2 (RNA extraction); 8.3.4 (reverse transcription), and 8.3.5 (qPCR). ABCA1 (Taqman probe Mm00442646.m1), ABCG1 (Taqman probe Mm00437390.m1) and SR-BI (Taqman probe Mm00450234.m1) gene expression were determined according to the manufacturer protocols. The reference gene used is HPRT1 (Taqman probe: Mm00446968.m1). HDL₂ used in the experiment was freshly dialyzed against PBS solution.

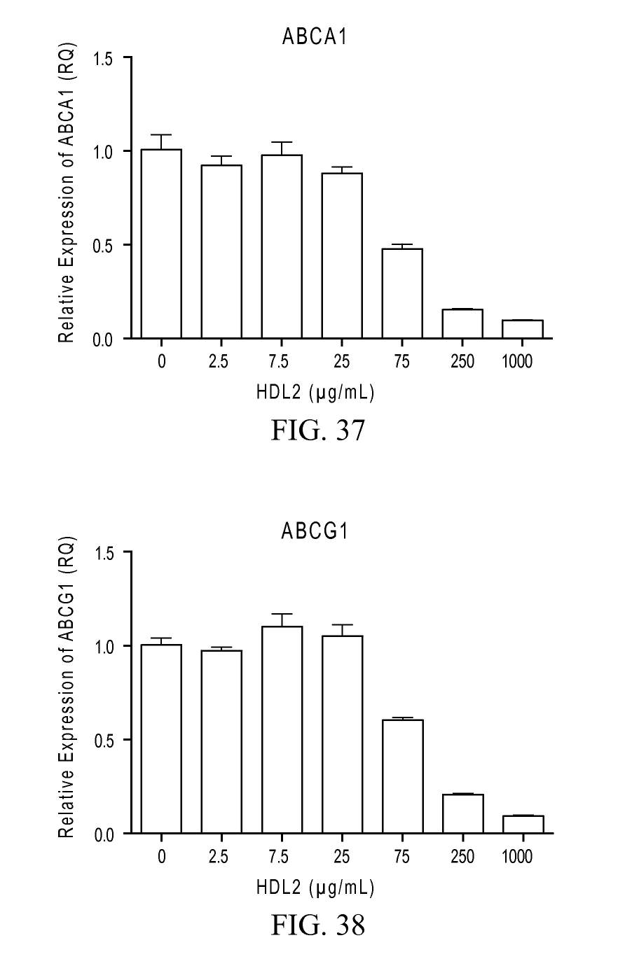

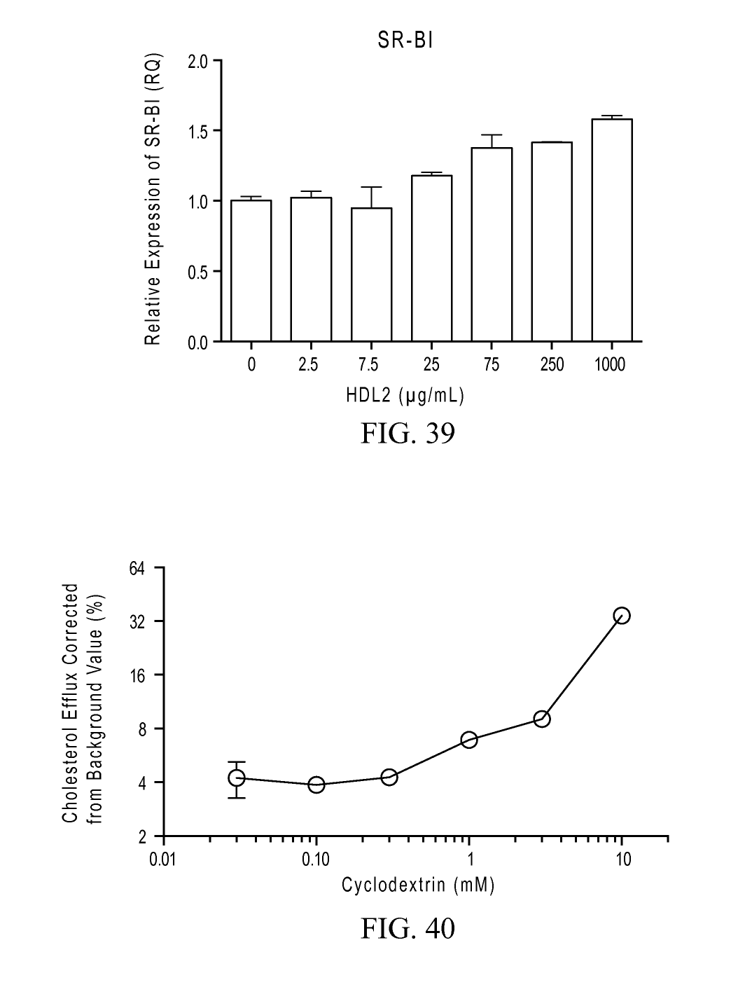

A significant decrease of ABCA1 and ABCG1 mRNA level in mouse macrophages was observed for HDL₂ treatment above 75 μg/mL (FIG. 37 and FIG. 38). SR-BI mRNA level starts to increase for HDL₂ concentration above 75 μg/mL (FIG. 39).

8.4.14. Study N: Regulation of ABCA1, ABCG1 and SR-BI mRNA Cellular Level by Cyclodextrin in J774 Macrophages—Determination of Cholesterol Efflux in Presence of Cyclodextrin

This study used β-cyclodextrin to examine whether intracellular cholesterol concentration could be responsible for the down-regulation of ABCA1 and ABCG1 in J774 mouse macrophages observed with CER-001 and HDL₃. β-cyclodextrin are cyclic oligosaccharides, soluble in water with a high specificity for sterols and able to efflux cholesterol from cells. J774 were seeded on 24 well plates (60,000 cells/well) and loaded with ³H-cholesterol oxidized-LDL in DMEM 2.5% FCS. After a 24 hour equilibration (DMEM), β-cyclodextrin (0.03, 0.1, 0.3, 1, 3, 10 and 30 mM) was added for 6 hours. The percentage of efflux, assayed using the protocol of Section 8.3.6, is determined as: Medium DPM/(Medium DPM+Cell DPM)*100. The 30 mM dose is not represented in the final graph as the dose was cytotoxic, killing half of the cell population.

A dose-dependent increase for cholesterol efflux with β-cyclodextrin was observed (FIG. 40).

8.4.15. Study 0: Regulation of ABCA1, ABCG1 and SR-BI mRNA Cellular Level by Cyclodextrin in J774 Macrophages—Determination of Gene Expression

This study used β-cyclodextrin to examine whether intracellular cholesterol concentration could be responsible for the down-regulation of ABCA1 and ABCG1 in J774 mouse macrophages observed with CER-001 and HDL₃. J774 were seeded on 6 well plates (300,000 cells/well). β-cyclodextrin (0.03, 0.1, 0.3, 1, 3, 10 and 30 mM) was added for 6 hours on the macrophages and the RNA were extracted with the RiboPure™ kit according to the manufacturer's protocol. Gene expression was assayed using the protocols described in Sections 8.3.2 (RNA extraction); 8.3.4 (reverse transcription), and 8.3.5 (qPCR). ABCA1 (Taqman probe Mm00442646.m1), ABCG1 (Taqman probe Mm00437390.m1) and SR-BI (Taqman probe Mm00450234.m1) gene expression were determined according to the manufacturer protocols. The reference gene used is HPRT1 (Taqman probe: Mm00446968.m1).

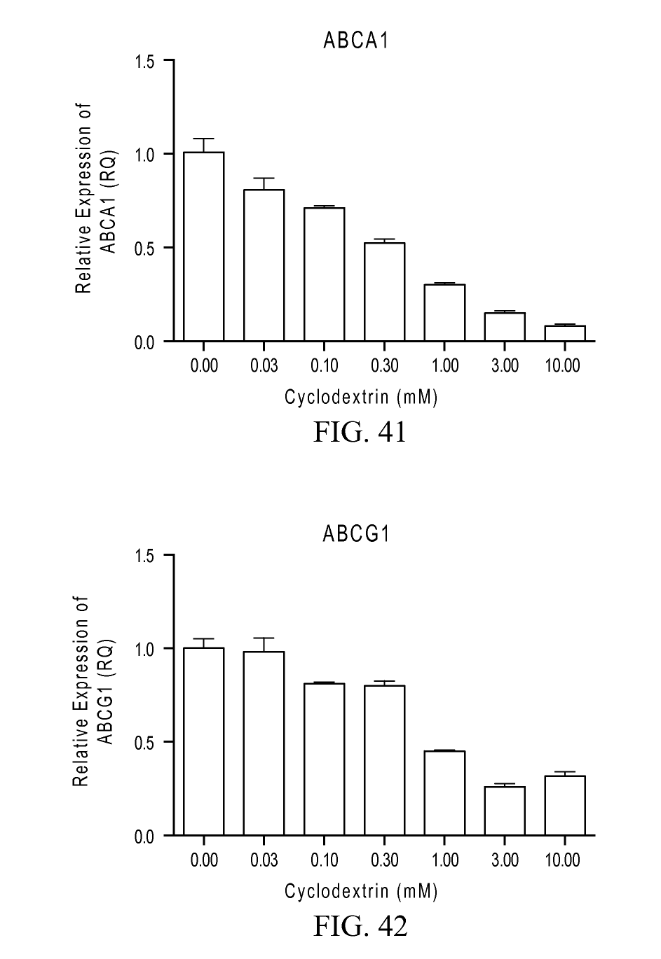

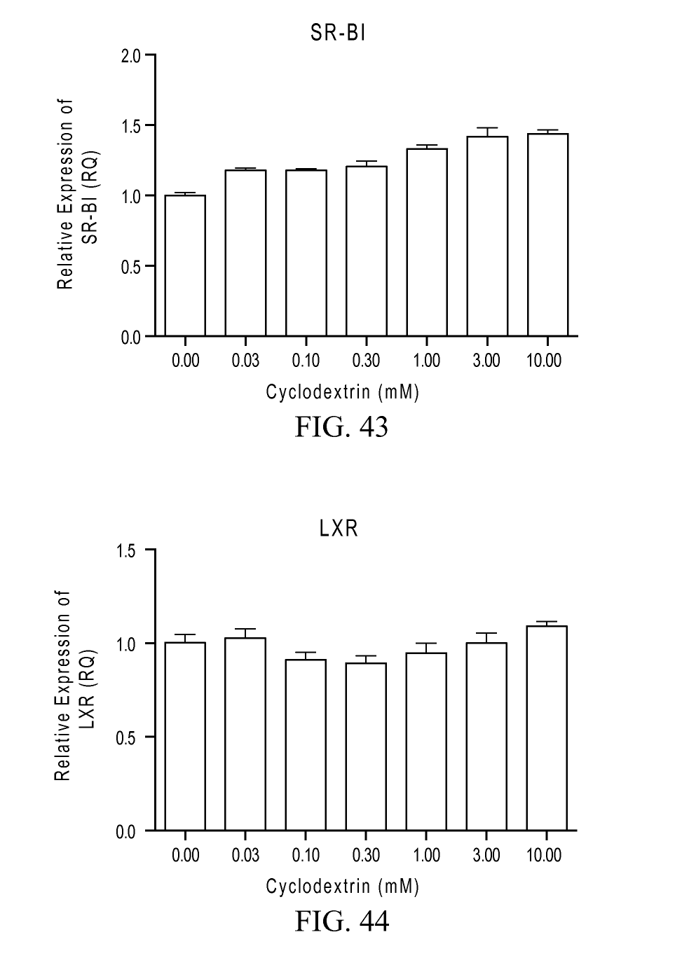

A dose-dependent decrease for ABCA1 and ABCG1 mRNA level in J774 was observed in the presence of β-cyclodextrin (FIG. 41 and FIG. 42). In contrast, SR-BI displayed a dose-dependent increase with β-cyclodextrin (FIG. 44).

8.4.16. Study P: Regulation of SREBP1, SREBP2 and LXR mRNA Cellular Level by Cyclodextrin in J774 Macrophages—Determination of Gene Expression

This study used β-cyclodextrin to examine the effect of β-cyclodextrin on LXR, SREBP1 and SREBP2 mRNA expression in J774 macrophages. J774 were seeded on 6 well plates (300,000 cells/well). β-cyclodextrin (0.03, 0.1, 0.3, 1, 3, 10 and 30 mM) was added for 6 hours on the macrophages and the RNA were extracted with the RiboPure™ kit according to the manufacturer's protocol. Gene expression was assayed using the protocols described in Sections 8.3.2 (RNA extraction); 8.3.4 (reverse transcription), and 8.3.5 (qPCR). SREBP-1, SREBP-2 and LXR gene expression were determined with Taqman probe (Mm01138344.m1, Mm01306292.m1, Mm00443451.m1 respectively) according to the manufacturer's protocol. The reference gene used is HPRT1 (Taqman probe: Mm00446968.m1).

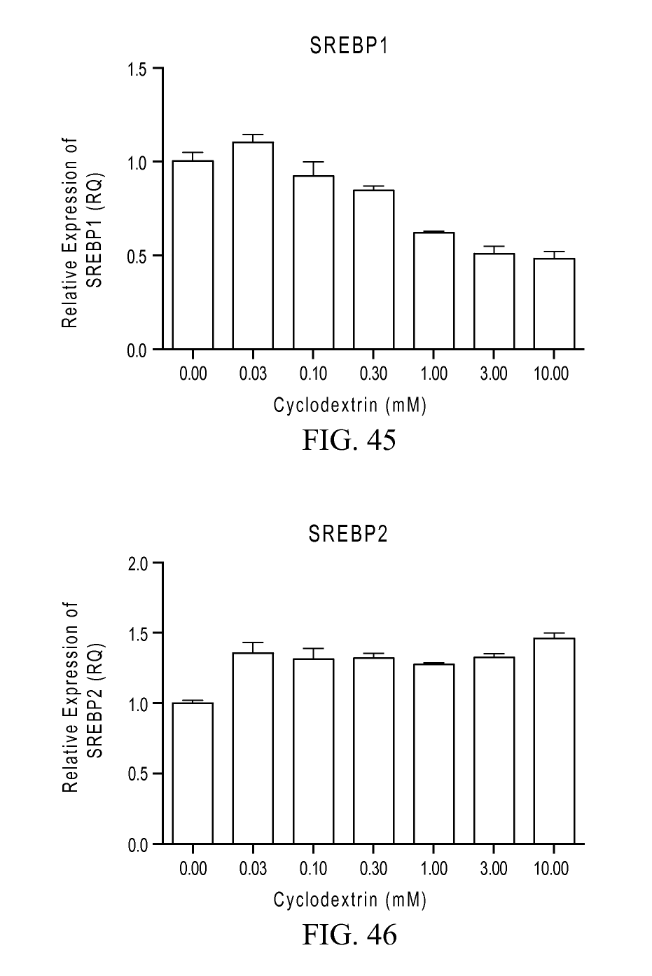

No significant changes were observed for LXR mRNA with increasing concentrations of β-cyclodextrin (FIG. 44). SREBP-2 mRNA increased for the lowest dose of β-cyclodextrin and reached a plateau (FIG. 46). A dose-dependent decrease for SREBP-1 mRNA (FIG. 45), similar to that observed with CER-001 and HDL₃ treatment, was observed.

### 9. EXAMPLE 3

### Measurement of Plaque Regression in APO^{-/-} Mice Flow Cessation Model Treated with CER-001

9.1. Introduction

The objective of studies A-F was to measure the efficacy of different CER-001 concentrations on plaque progression in ligatured left carotid from apoE^{-/-} mice fed with a high fat diet.

9.2. Materials & Methods

9.2.1. Overview

The materials used for these studies included CER-001 (1109HDL03-2X240913 batch concentrated by a membrane Vivaflow 30 KDa cassette) and purified human HDL₃. Prior to the experiment, CER-001 and HDL₃ formulations were aliquoted in at least 8 aliquots/lipoprotein concentration (1 aliquot used per group injection). Prior to injection, one aliquot of formulation was thawed by incubating in a ca. 37° C. water bath for 5 minutes and swirled gently. The formulation should not be shaken or vigorously agitated to avoid foaming. If the solution was turbid or if visible particulates were observed, the solution was incubated in a water bath at ca. 37° C. for an additional half hour.

Phosphate buffered sucrose diluent (10 mMPhosphate, 4% sucrose and 2% mannnitol, pH=7.4) was prepared, aliquoted and stored at ca. −20° C. The placebo solution was used for the preparation of the different concentrations of CER-001 and HDL₃.

9.2.2. Animals

The animals used in these studies were mice of the strain C57BI/6-B6.129P2-Apoetm1 Unc/J. The strain comes from the Jackson laboratory and is distributed by Charles River. This specie and strain is a well characterized model for the study of cholesterol metabolism. Inclusion criteria included weight: 21 grams (8 week old), 23 grams (9 week old) and 25 grams (12 week old); age: 8, 9 and 12 weeks at the start of the diet and sex and number: male, n=125 (12 mice per group).

The animals were housed in the animal facilities of Prolog Biotech by groups of maximum 12 animals/cage. Prolog Biotech has the agreement number A-31-254-01 obtained from the French Veterinary Authorities. In each cage, 2 igloos were added to the well-being of animals. The animals were acclimated 5 days before beginning of the study (from 9/18 to 9/23). The animals had access to water and a high cholesterol diet (0.2% cholesterol, 39.9% fat, 14.4% proteins, 45.7% sugars). All animals were managed similarly and with due regard for their well-being according to prevailing practices of the animal facility of Prolog Biotech. The study plan has been accepted by the Prologue Biotech Ethical Committee (N^{o} CEF-2011-CER-09).

The animal room conditions were as follows: temperature: 21±1° C., relative humidity: 50±10% and light/dark cycle: 12 h/12 h (07H/19H). Each month a report on animal room conditions is edited. Each animal was weighted every week. Animals were identified by earrings inserted at the beginning of the experiment.

9.2.3. Treatment

The animals were divided into 10 groups with 12 animals per group and treated as indicated in Table 4.

TABLE 4

| | | Dose level | Number of | Number of |
|---|---|---|---|---|
| Group | Formulation Id. | (mg/kg) | days on HFD | infusions |
| 1 | Placebo | 0 | 22 | 8 |
| 2 | CER-001 | 2 | 22 | 8 |
| 3 | CER-001 | 5 | 22 | 8 |
| 4 | CER-001 | 10 | 22 | 8 |
| 5 | CER-001 | 20 | 22 | 8 |
| 6 | CER-001 | 50 | 22 | 8 |
| 7 | HDL₃ | 5 | 22 | 8 |
| 8 | HDL₃ | 10 | 22 | 8 |
| 9 | HDL₃ | 20 | 22 | 8 |
| 10 | HDL₃ | 50 | 22 | 8 |

The formulation was injected in the retro-orbital vein (50 μL/mouse) of mice anaesthetized with isoflurane, once every 2 days for 8 injections. The dose administered was based on the mean of mice bodyweight in each cage. The compounds were injected at 10 AM every day. For blood sampling, mice fasted overnight were sampled once at the indicated dose: (1) at predose (at 9 AM) by retro-orbital withdrawal: 24 hours before the first injection/day of surgery; (2) at postdose (at 9 AM) by retro-orbital withdrawal: 24 hours after the last injection; and (3) at t=0 (9 AM) and the indicated time points after the 5^{th} injection by caudal withdrawal. Immediately after collection, blood samples were kept at ca. +4° C. to avoid alteration of the blood sample. Blood specimens were centrifuged (800×g for 10 minutes at +4° C.) and plasma was saved for future analysis.

9.2.4. Surgery

For organ collection, 24 hours after the last injection, mice were anesthetized with a mix of ketamine (100 mg/kg) and xylazine (10 mg/kg) injected intraperitoneally and the animals fell asleep after 2 or 3 minutes. Blood was withdrawn by capillarity (retro-orbital vein —approximately 200 μl of blood) and transferred to a tube containing EDTA. Then an abdomino-thoracic incision was done. The heart was perfused with PBS by the right ventricle to do a first wash and if necessary by the left ventricle. The liquid should have flowed by the thoracic aorta.

The left and right carotids, the liver, the spleen and the aortas connected to the heart were removed and stored at −80° C. The liver was collected in four different aliquots. The remaining biological specimens were discarded after the organ collection. For feces collection, the day of the last injection for each group, the cage was changed with a new litter and feces were collected for 24 hours (day of sacrifice).

9.2.5. Determination of Total Plasma Cholesterol

Experimental Procedure:

Add in each tube cholesterol standards (2 g/L): 0/0.625/1.25/1.875/2.5/3.75/5 μl. Centrifuge plasma samples at 12,000×g for 1 minute. Depending on the species, add samples into each tube as shown in Table 5. Add 0.5 ml of the reconstituted buffer to each tube (standard and samples), vortex and incubate 5 minutes at 37° C. Transfer 150 μl from each tube to 2 different wells in a 96 well plate. Read absorbance at 500 nM.

TABLE 5

| | Volume (μl) | Volume (μl) | Volume (μl) |
|---|---|---|---|
| | Plasma | Plasma | Plasma |
| Species | Day 0 | Day 7 | Day 14 |
| Mouse | 5 μl | 5 μl | 5 μl |
| C57BL/6J | | | |
| Mouse ApoE-/- | 20 μl | 10 μl | 10 μl |
| KO | of a 1/10 diluted | of a 1/10 diluted | of a 1/10 diluted |
| | samples in H₂O | samples in H₂O | samples in H₂O |
| Rabbit | 7.5 μl | 7.5 μl | 7.5 μl |

9.2.6. Determination of Non Esterified Cholesterol in Plasma Experimental Procedure:

Add in each tube cholesterol standards (2 g/L): 0/0.625/1.25/1.875/2.5/3.75/5 μl. Centrifuge plasma samples at 12,000×g for 1 minute. Depending on the species, add samples into each tube as shown in Table 6. Add 0.5 ml of reconstituted buffer to each tube (standard and samples), vortex and incubate 5 minutes at 37° C. Transfer 150 μl from each tube to 2 different wells in a 96 well plate. Read absorbance at 500 nM.

TABLE 6

| | Volume (μl) | Volume (μl) | Volume (μl) |
|---|---|---|---|
| Species | Day 0 | Day 7 | Day 14 |
| Mouse C57BL/6J | 10 μl | | |
| Mouse ApoE-/-KO | 5 μl | 2.5 μl | 2.5 μl |
| Rabbit | 20 μl | | |

9.2.7. Determination of Cholesterol by RP C18 HPLC

Equipment: HPLC (Waters Binary HPLC pump 1525, Waters UV/Visible detector 2489, Waters Sample manager 2767, Masslynx software (4.1), column: RP C18 Zorbax 4.6 mm×25 cm, particle size 10 μm (or equivalent), acetonitrile HPLC grade, absolute ethanol, water (milliQ), standard cholesterol 0.1 g/l in absolute ethanol.

Chromatography Parameters:

Eluent A: 86% acetonitrile, 10% ethanol, 4% water; Eluent B: 86% acetonitrile, 14% ethanol. Sonicate the eluent 5 min in the sonicator bath before use. Flow rate: 1.5 mL/min; Pressure: 1400 PSI; detection: UV 214 nm; run time: 20 min; injection: 25 to 100 μL; gradient program shown in Table 7:

TABLE 7

| Time mn | Flow rate ml/mn | % A | % B |
|---|---|---|---|
| 0 | 1.5 | 100 | 0 |
| 10 | 1.5 | 100 | 0 |
| 11 | 1.5 | 0 | 100 |
| 12 | 1.5 | 0 | 100 |
| 13 | 1.5 | 100 | 0 |
| 35 | 1.5 | 100 | 0 |
| 36 | 0 | 100 | 0 |

Samples:

Samples are prepared according to the method of Section 9.2.8. Add 50 μl of ethanolic extract into micro vials and inject 40 μl into the HPLC. Determine the Peak area at 214 nm and calculate the concentration in the extract: [Cholesterol sample] (μg/μl)=peak area/slope/injected volume

9.2.8. Cholesterol Extraction from Livers

Step 1:

Weigh˜50 mg of liver, introduce the tissue in a glass tube. Homogenize (Turrax®) the tissue in 3 ml MeOH.

EDTA 5 mM (2:1). Add 3 ml of chloroform and 3 ml of H₂O and vortex for five minutes. Centrifuge 10 min at 1,500×g and collect the lower phase. Split the solution in 2 glass tubes (small) in equal volumes (2×1.3 ml).

Step 2:

Treat solutions as follows:

Unesterified cholesterol: Dry the solution. Add 400 μl EtOH for solubilisation of the sample.

Total cholesterol: Dry the solution. Add 1 ml methanolic KOH solution 0.5M. Incubate at 60° C. for at least 1 hour. Perform a Bligh and Dyer lipid extraction by adding 1 ml of chloroform and 1 ml of H₂O to the sample, vortexing, centrifuging for 10 min at 1,500×g and collecting the lower phase. Dry the organic phase. Add 400 μl EtOH for solubilisation of the sample.

9.2.9. Cholesterol Extraction from Carotids or Aortas

Step 1:

Remove the surgical straps (only for the carotids) and introduce the tissue in a glass tube. Add 1.8 ml of CHCl₃/MeOH (2:1) for the carotids or 3 ml for the aortas. Mix overnight at 4° C.

Step 2:

Remove, dry and weigh the carotid or aorta. Split the organic solution (CHCl₃/MeOH) in 2 glass tubes (small) in equal volumes. Treat solutions as follows:

Unesterified cholesterol: Dry the solution. Add 200 μl EtOH for solubilisation of the sample.

Total cholesterol: Dry the solution. Add 1 ml ethanolic KOH solution 0.1M. Incubate at 60° C. for at least 1 hour. Perform a Bligh and Dyer lipid extraction by adding 1 ml of chloroform and 1 ml of H₂O to the sample, vortexing, centrifuging for 10 min at 1,500×g and collecting the lower phase. Dry the organic phase. Add 200 μl EtOH for solubilisation of the sample.

9.3. Results of In Vivo Plaque Progression Studies A-F

9.3.1. Study A: Determination of Atherosclerotic Plaques in Ligatured Left Carotids

This study examined the effect of CER-001 administration on plaque progression in ligatured carotid from apoE^{-/-} mice fed with a high fat diet. For each group of mice, the cholesterol content of the carotid was tested after lipid extraction and HPLC analysis. The ligatured carotids were collected the day of sacrifice and stored at −80° C. The lipids were extracted with an organic solution and the cholesterol concentration was determined by HPLC.

Ligatured carotids were lipid extracted according to the method of Section 9.2.9. The surgical straps were removed from the carotid (fresh weight) and the tissue was introduced in a glass tube. To this was added 1.8 mL of CHCl₃/MeOH (2:1) and mixed overnight at 4° C. The carotid was then removed, dried and weighed and the organic solution (CHCl₃/MeOH) was split in two glass tubes in equal volumes. For unesterified cholesterol (UC), 100 μL of β-sitosterol (internal standard) was added and the solution was dried. To this was added 200 μL EtOH for solubilisation of the sample and the sample was analyzed UC by HPLC. For total cholesterol (TC), 100 μL of β-sitosterol (internal standard) was added and the solution was dried. To this was added 1 mL methanolic KOH solution 0.1M and the solution was incubated at 60° C. for at least 30 minutes. A Bligh and Dyer procedure was performed for cholesterol extraction wherein 1 mL of chloroform was added, followed by 1 mL of H₂O and mixing by vortex. When the phases separated, the lower phase was collected and dried. To this was added 200 μL EtOH for solubilisation of the sample and analysed TC by HPLC. The cholesterol concentration was determined by HPLC according to the method of Section 9.2.7. Briefly, 50 μL were injected on a C18 Zorbax: SB-C18 4,6×250 mm (Agilent ref 880975-902) column. Flow rate was 1.5 mL/min at 60% of Eluent A (ACN/ETOH/H₂O 85/10/5) and 40% of Eluent B (ACN/ETOH 86/14). The run time was 55 min with a retention time for cholesterol at 22.85 min and a retention time for β-sitosterol of 32.2 min. System: Binary pump Waters 1525—UV detector set at 214 nm—Software: Massslynx 4.1.

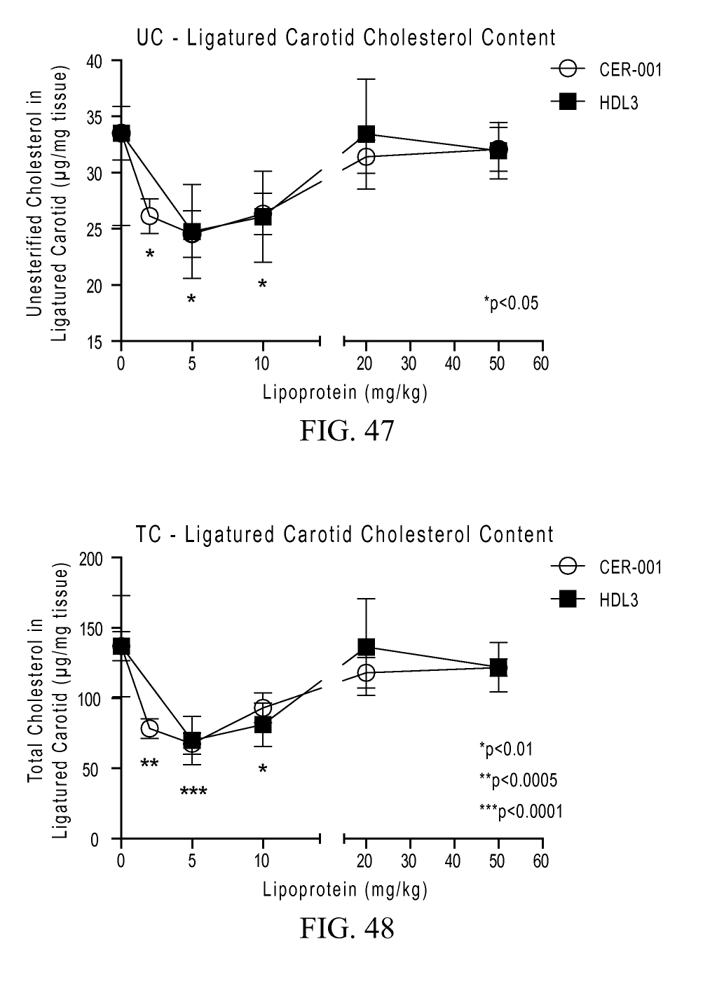

A similar profile for cholesterol content in ligatured carotids for the mice treated with CER-001 or HDL₃ was observed (FIG. 47 and FIG. 48). For concentrations of 2, 5 and 10 mg/kg, a 25% decrease in unesterified cholesterol was observed and a 50% decrease in total cholesterol contents in ligatured carotids was observed. The inhibition of plaque progression for the doses of 20 and 50 mg/kg is around 10% for treatments with CER-001 and HDL₃.

9.3.2. Study B: Determination of Plasma Cholesterol Mobilization after CER-001 Infusion

This study examined the consequences of CER-001 administration on the lipoprotein profile in apoE^{-/-} mice fed with high fat diet. Blood was collected and analyzed at different time points after the 5^{th} infusion. Plasma pre-dose (before the first injection) and plasma post-dose (24 h after the last injection) were also compared. Plasma was analyzed for total and unesterified cholesterol and human ApoA-I contents.

Total and unesterified cholesterol concentrations were determined according to the procotols of Sections 9.2.5 and 9.2.6, respectively. Cholesterol ester concentrations are determined after subtracted unesterified cholesterol from total cholesterol. The mobilization of cholesterol was determined on 12 mice per group after the 5^{th} administration (1 hour before injection; 1 h, 2 h, 4 h and 24 h after injection). The animals were fasted overnight.

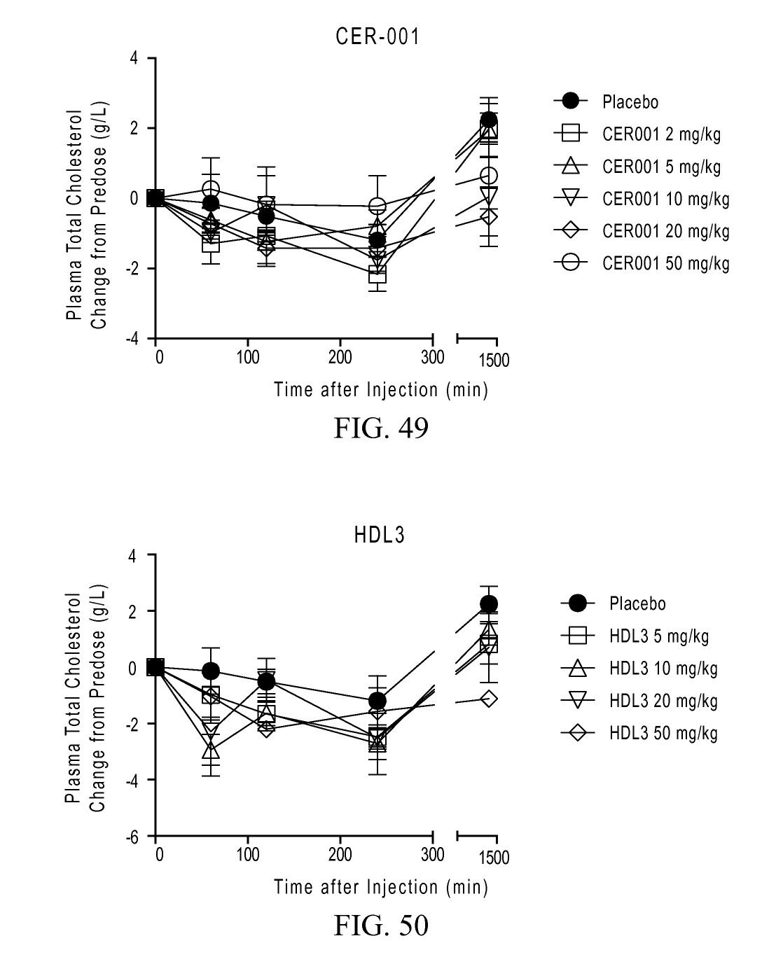

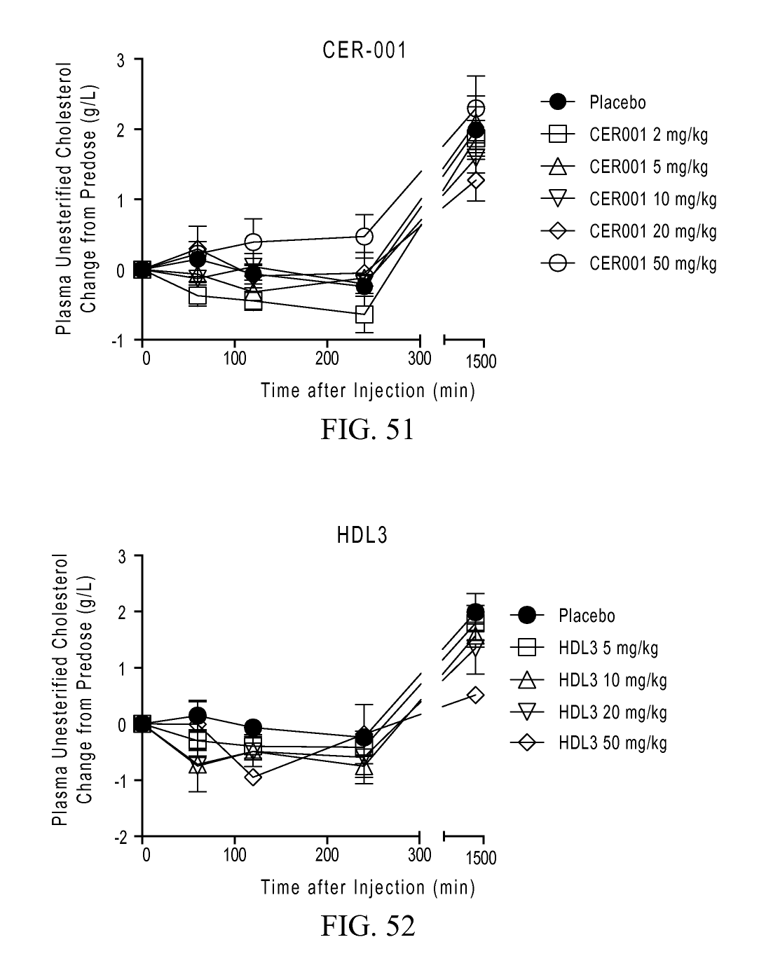

No significant changes in total plasma cholesterol mobilization were observed after infusion of CER-001 or HDL₃ (FIG. 49 and FIG. 50). No significant changes in mobilization of unesterified cholesterol were observed after CER-001 and HDL₃ infusion (FIG. 51 and FIG. 52). CER-001 at 50 mg/kg seemed to increase the plasma unesterified cholesterol concentration at 2 and 4 hours after infusion.

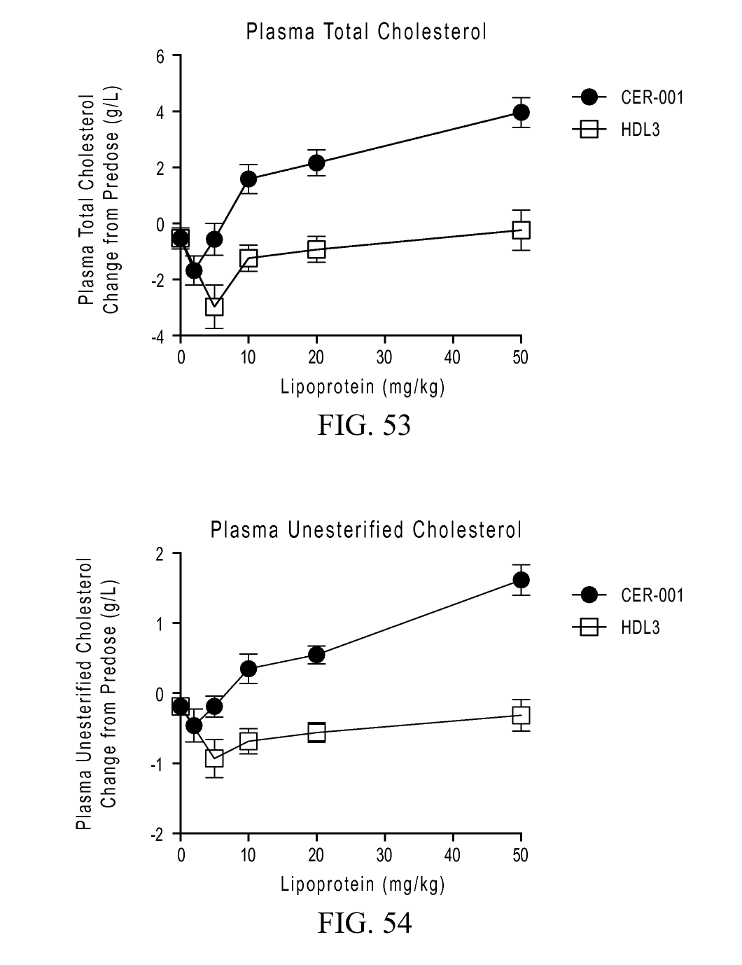

The post-dose profiles for CER-001 and HDL₃ were similar except the total and unesterified cholesterol concentrations were two times higher in CER-001 treated animals compared to HDL₃ treated mice (FIG. 53 and FIG. 54). Doses above 10 mg/kg for CER-001 increased the cholesterol concentration in mouse plasma after 8 injections compared to placebo. HDL₃ infusion did not increase the cholesterol concentration above the placebo.

9.3.3. Study C: Determination of Plasma Human ApoA-I

This study examined the kinetics of the CER-001 infusion by determining the concentration of human ApoA-I in the plasma after infusion of CER-001. The ApoA-I concentration in plasma was determined by ELISA (Assay Pro EA5201-1) following manufacturer instructions. Prior to ApoA-I determination the plasma were diluted 1/100, 1/50 or 1/10 depending on CER-001 and HDL₃ concentrations injected to the mice.

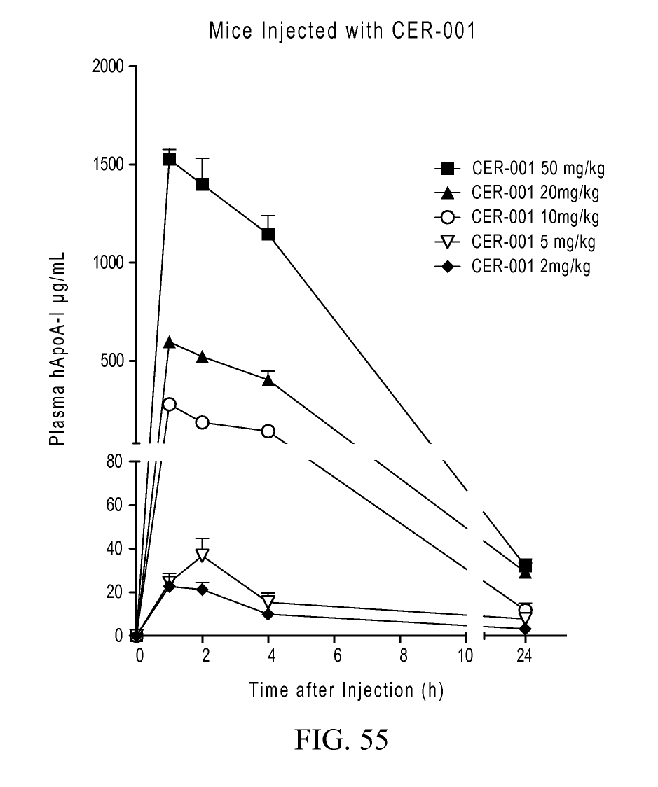

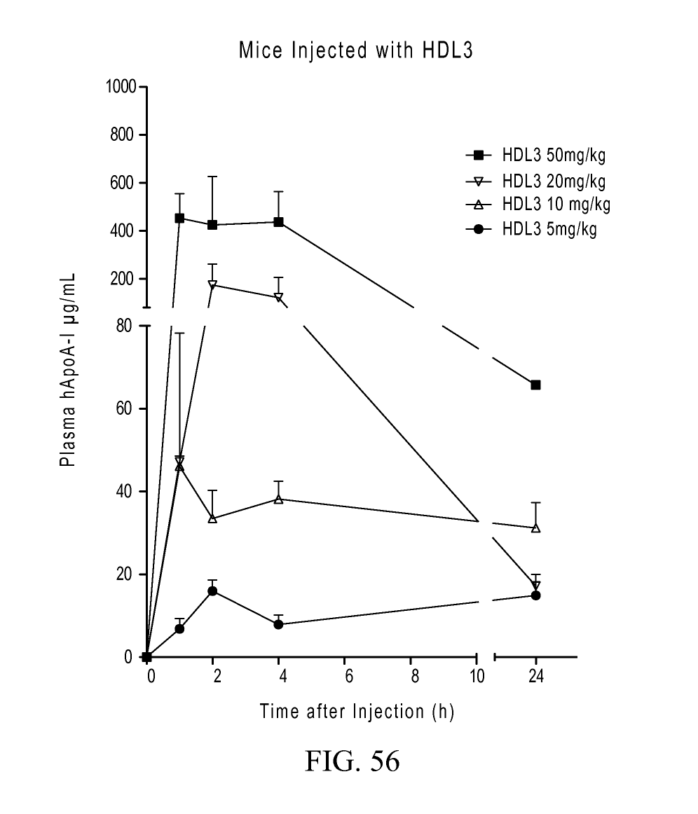

A dose-dependent increase in human ApoA-I plasma concentration was observed with CER-001. The expected doses of ApoA-I in plasma were retrieved for all the concentrations tested (FIG. 55). For HDL₃, a dose-dependent increase in human ApoA-I plasma concentration was observed (FIG. 56). However, the human plasma ApoA-I is three times less concentrated than the expected doses.

9.3.4. Study D: Western Blot Determination of ABCA1 Expression in Ligatured Carotids

This study examined if the expression of ABCA1 could be related to the difference in cholesterol content as a decrease (5 mg/kg CER-001) and no effect (50 mg/kg CER-001) was observed in cholesterol content in mouse ligatured carotids. Ligatured carotids previously extracted with chloroform:methanol were solubilized in NAOH 0.1N (100 μL/carotid). The solution was briefly sonicated and centrifuged at 15,000×g for 10 minutes. The protein concentration was determined with Bradford assay and 40 μg of sample were loaded on SDS-PAGE. The ABCA1 expression (ab7360—dilution 1/1000) was quantified using imageJ® software.

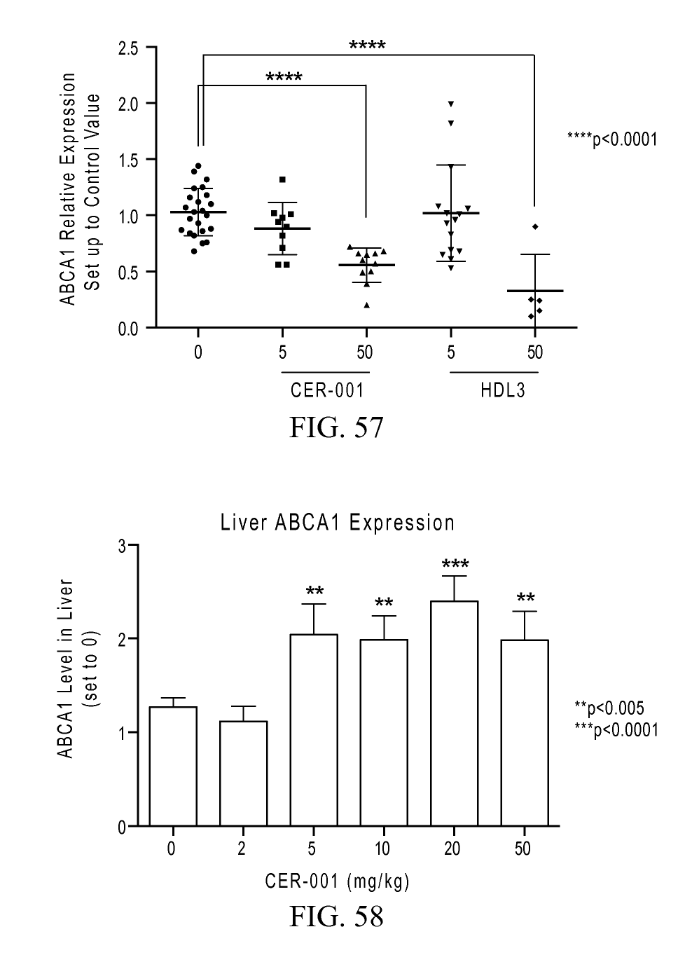

A decrease for carotid ABCA1 content was observed for CER-001 and HDL₃ at 50 mg/kg dose (FIG. 57). The 5 mg/kg dose did not affect the ABCA1 expression for both CER-001 and HDL₃. The ABCA1 expression in ligatured carotid was down-regulated for 50 mg/kg CER-001 and HDL₃ dose. Cholesterol efflux for those macrophages may have been impaired which could explain the absence of effect on plaque cholesterol content for concentrations of 50 mg/kg.

9.3.5. Study E: Determination of SR-BI and ABCA1 in Mouse Liver

This study examined the SR-BI and ABCA1 protein content in the liver 24 hours after the last injection of CER-001. A piece of liver (50 mg) was lysed in PBS (500 μL) by brief sonication. The sample was centrifuged (800×g for 10 minutes) and the pellet was discarded. The supernatant was centrifuged for 30 minutes at 16,000×g at 4° C. and the pellet was solubilized with PBS 1% Triton X100 (200 μL). 10 μg of solubilized pellet was loaded on SDS PAGE 10% and ABCA1 expression (ab7360—dilution 1/1000) or SR-BI expression (ab24603—dilution 1/1000) was quantified using imageJ® software.

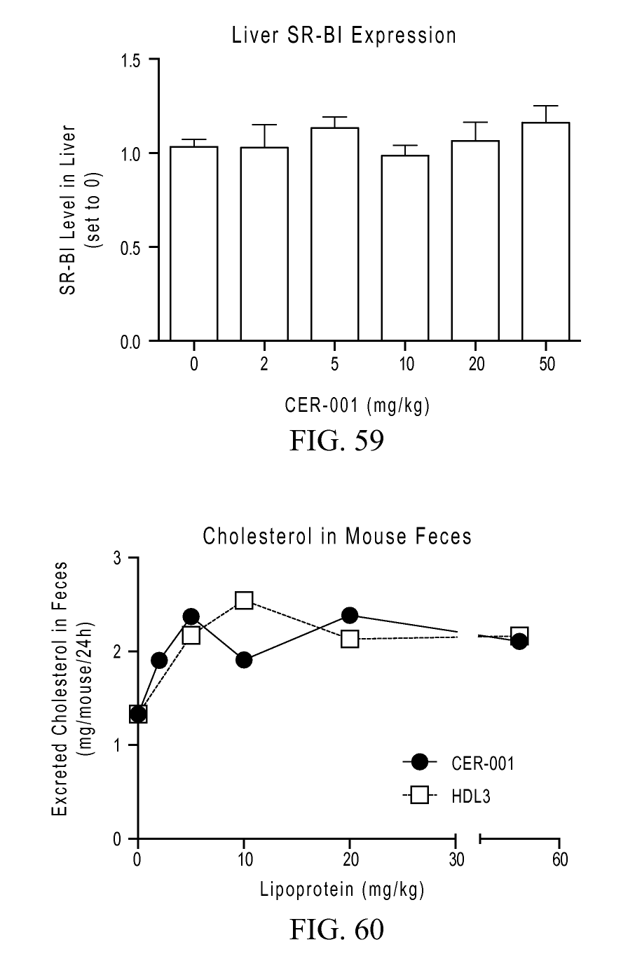

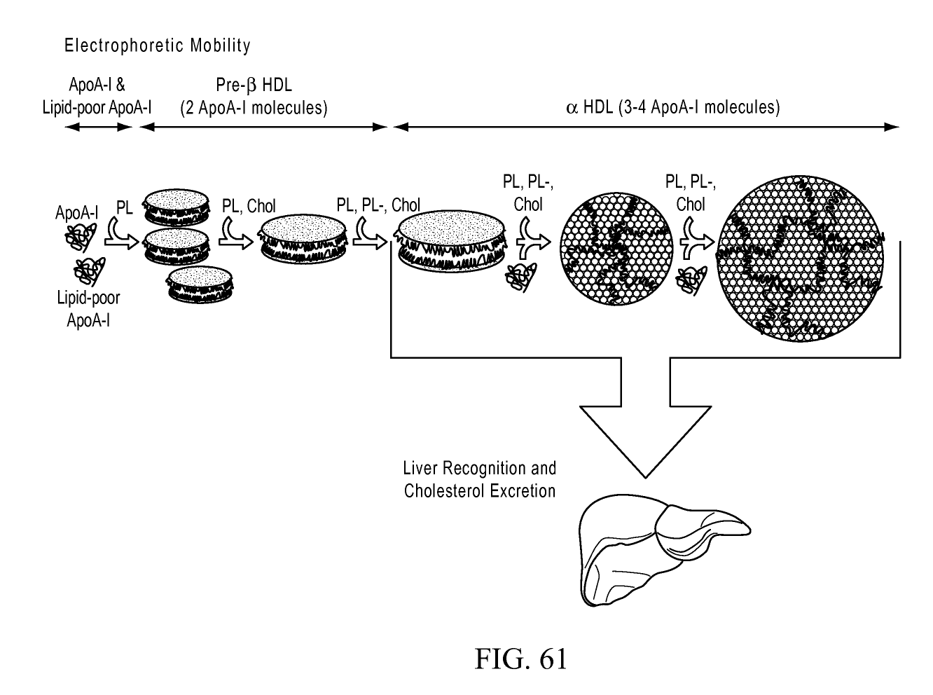

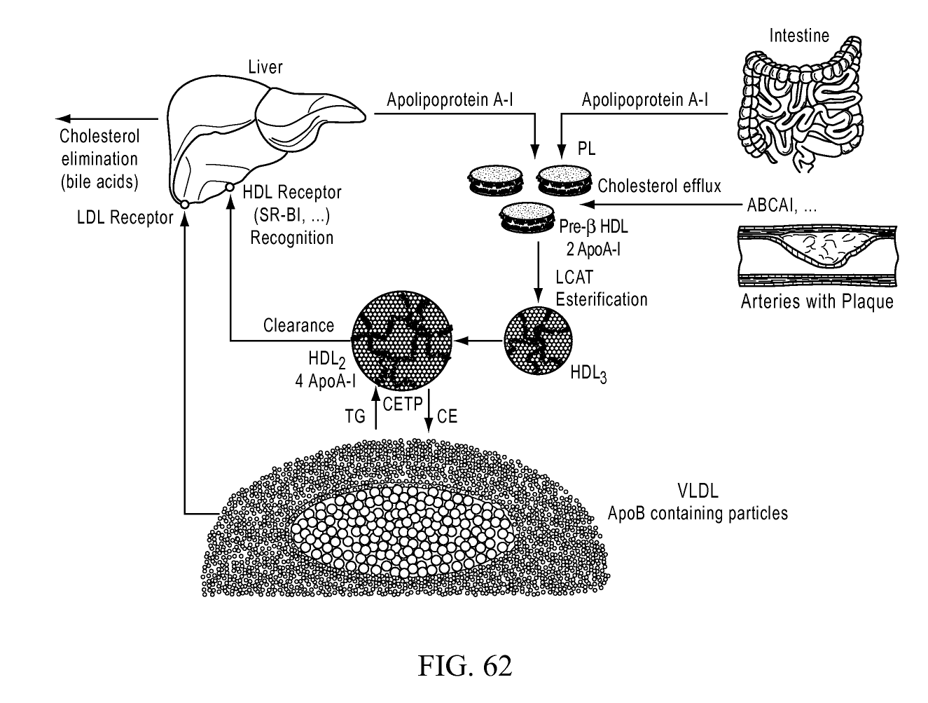

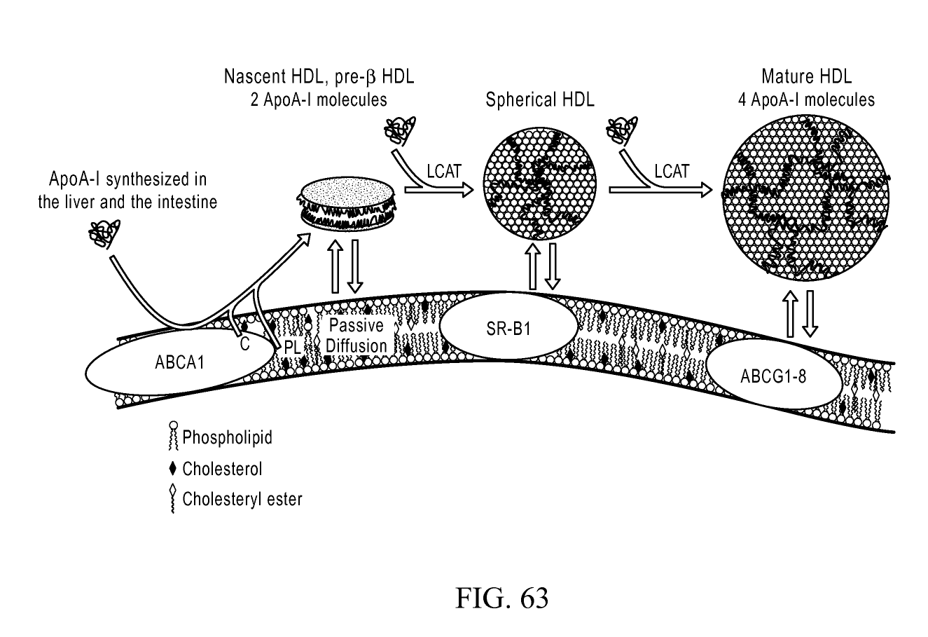

In contrast to the ABCA1 carotid content, an increase in ABCA1 protein level was observed in the mouse liver with increasing concentrations of CER-001 (FIG. 58). This discrepancy could be explained by: i) the cell population was different; in carotids the cell population is composed of macrophages and endothelial cells; in liver the cell population is in majority hepatocytes, ii) the form of CER-001 and its function are different in both cases; for carotid CER-001 is poorly charged in cholesterol and its function is to efflux cholesterol from cells; for liver, CER-001 is cholesterol loaded and its function is to be eliminated by the liver. Because ABCA1 expression is tightly regulated by cholesterol content, we hypothesized that in cholesterol poor environment (high cholesterol efflux for example), the ABCA1 expression is decreased and in cholesterol rich environment (cholesterol uptake), the ABCA1 expression is increased. For SR-BI no significant changes were observed for protein level with increasing concentrations of CER-001 (FIG. 59).

9.3.6. Study F: Determination of Cholesterol Content in Mouse Feces

This study analyzed the cholesterol content in mouse feces for different concentrations of CER-001 and HDL₃. Feces were lipid extracted and analyzed by HPLC for their cholesterol content. Feces (200 mg) were solubilized in methanol:water (50:50) solution and mixed for 1 minute with Turrax®. The solution was frozen and lyophilized overnight. The following day, 4 mL of chloroform/methanol (2:1) was added and the solution was mixed for 24 hours at 4° C. To this was added water (1.33 mL), and the solution was then mixed and centrifuged for three minutes at 3700×g. The organic phase was saved and dried. The pellet was solubilized in absolute ethanol (2 mL), and filtered on cartridge AC 0.2 μm. The cholesterol concentration in the sample was analyzed according to the method of Section 9.2.7.

A dose-dependent increase in feces cholesterol content was observed for mice injected with CER-001 and HDL₃ (FIG. 60). Maximum cholesterol excretion was observed for a concentration of 10 mg/kg.

### 10. EXAMPLE 4

### Clinical Testing of CER-001 in Patients with Hypoalphalipoproteinemia

10.1. Background

Cerenis has completed some early clinical trial work in subjects with hypoalphalipoproteinemia due to genetic defects (including a Tangier disease patient and two ABCA1 heterozygotes).

The burden of cholesterol trapped in vessel walls throughout the body because of a lifelong deficit in the RLT pathway should be reduced incrementally with each iterative dose during an “induction phase,” and atherosclerotic plaque should regress in patients in whom LDL levels are adequately controlled. Therapy would continue chronically at a reduced dosing interval (“maintenance phase”) in order to maintain appropriate cholesterol homeostasis—i.e., a balance between delivery to the tissues by the endogenous LDL-C and removal by the infused CER-001 pre-3-like HDL particle. CER-001 therapy could be lifelong, since the inherent defect in HDL production and RLT, by virtue of the genetic causality, is permanent.

Table 8 below shows the profiles of the patients included in the trial (called the SAMBA trial).

TABLE 8

| | | Baseline | Baseline | | |
|---|---|---|---|---|---|
| | | HDL-c | ApoA-I | | Lipid control |
| Subject | Genotype | (mg/dL) | (mg/dL) | CV history | meds |
| 001 M/46 | ApoA-I −/− | 1.8 | 1.8 | CABG | Atorvastatin |
| | | | | | 80 mg |
| | | | | | Ezetimibe |
| | | | | | 10 mg |
| | | | | | Niacin |
| 002 M/55 | ABCA1 +/− | 19.7 | 28.7 | MI × 3 | Rosuvastatin |
| | | | | PCI | 15 mg |
| 003 M/49 | ABCA1 +/− | 6.2 | 16.5 | MI | Rosuvastatin |
| | ApoA-I +/− | | | PCI | 10 mg |
| 004 M/51 | LCAT +/− | 29.0 | 59.1 | MI × 2 | Simvastatin |
| | | | | | 40 mg |
| | | | | | Ezetimibe |
| | | | | | 10 mg |
| 005 M/68 | ABCA1 +/− | 13.8 | 51.6 | Hypertension | None |
| 006 F/51 | ApoA-I +/− | 37.4 | 70.2 | None | None |
| 007 F/47 | ABCA1 −/− | 0.6 | 7.9 | PCI | Aspirin |
| | | | | | Rosuvastatin |
| | | | | | 10 mg |
| | | | | | Ezetimibe |
| | | | | | 10 mg |

The patients were initially treated in an intense “induction phase,” receiving 9 doses of CER-001 at a dose of 8 mg/kg over 4 weeks. After this induction phase, the study subjects were re-evaluated with lipoprotein profiles and an MRI scan. Subsequently, the study subjects continued to be treated once every two weeks in a “maintenance phase” for 6 months' total therapy. At that point the lipoprotein profiles and MRI scans were repeated.

10.2. Results

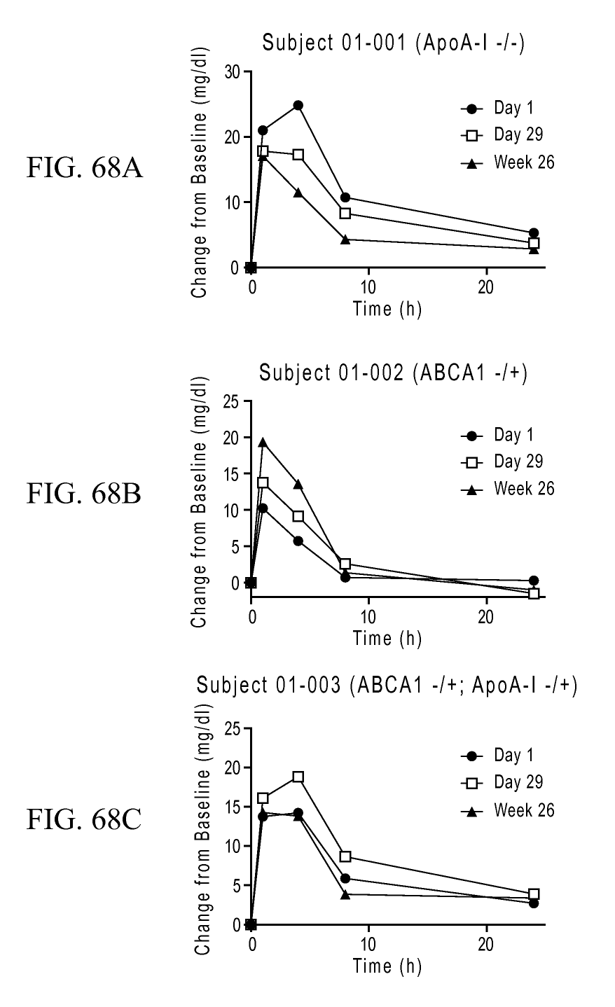

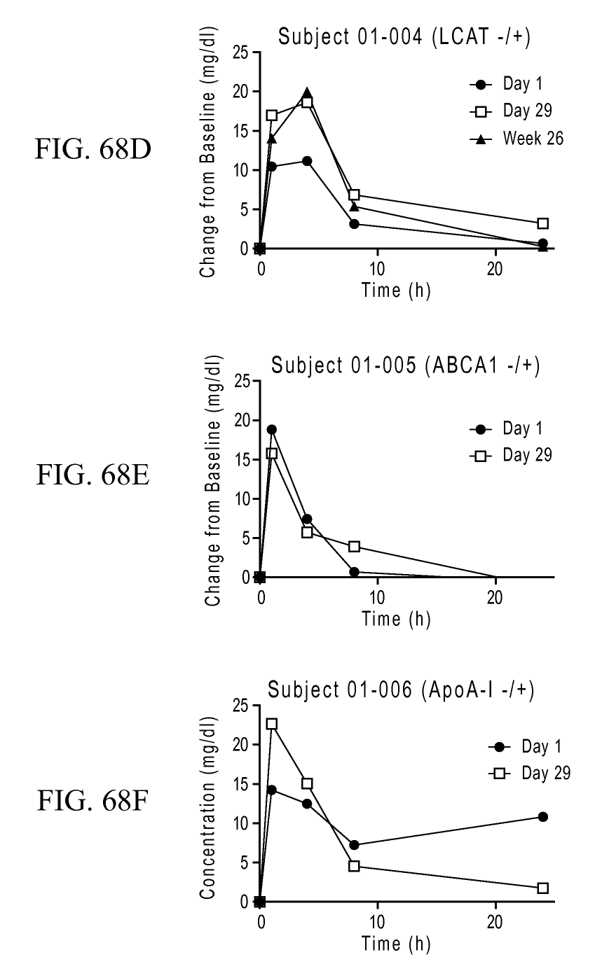

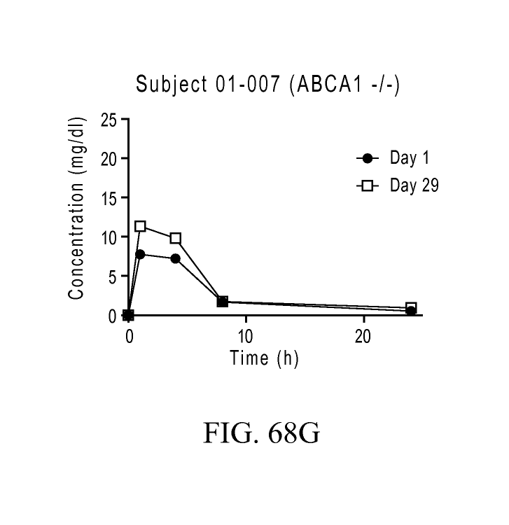

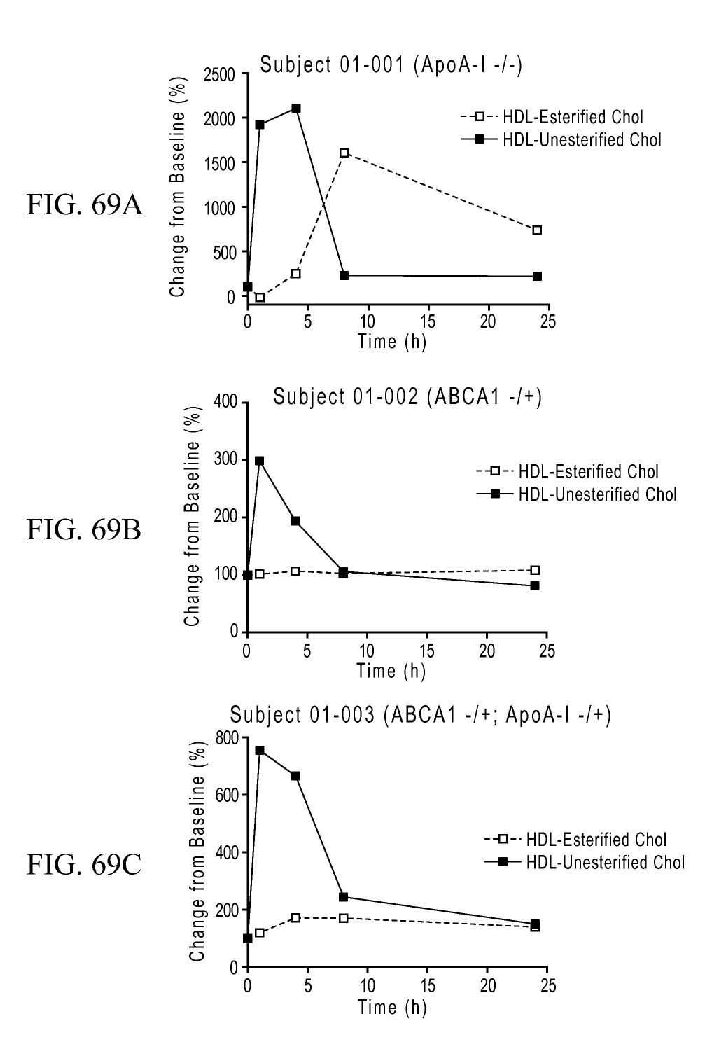

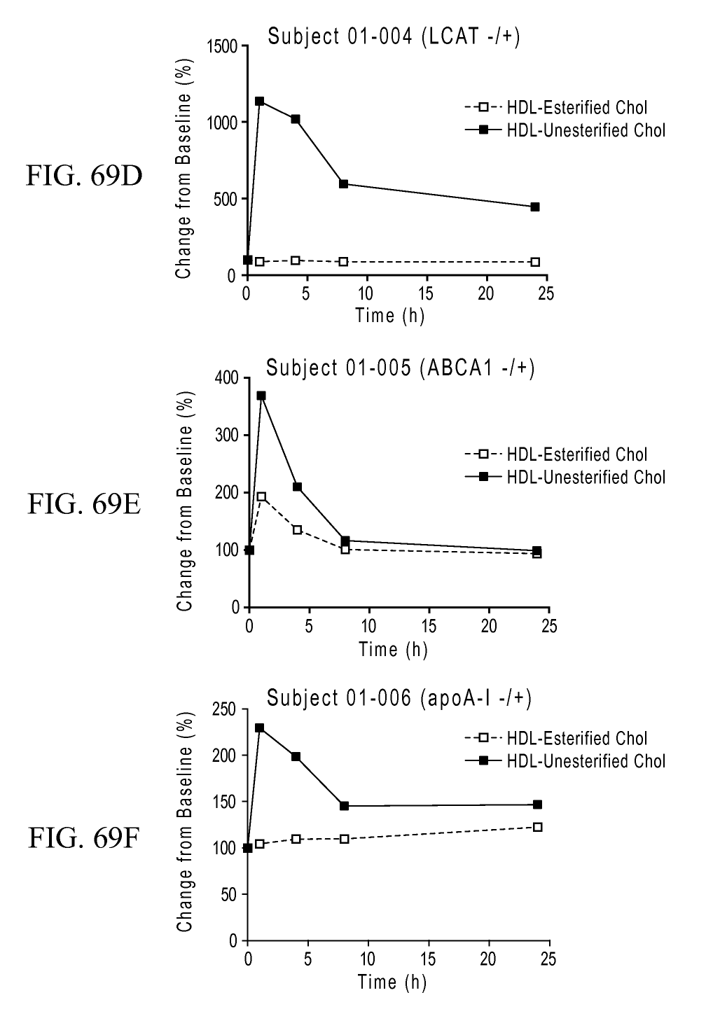

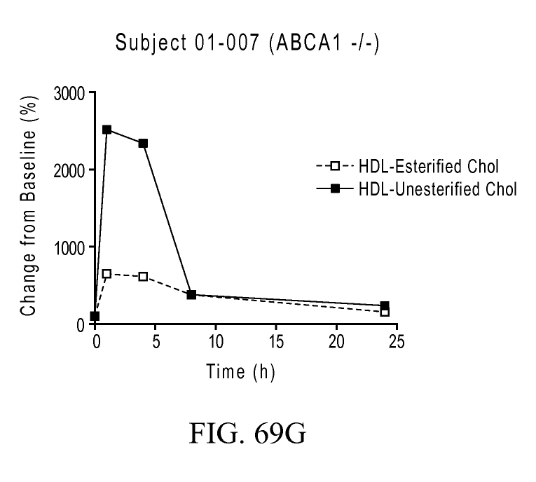

The effects of CER-001 on cholesterol mobilization and cholesterol esterification by LCAT are shown on a subject-by-subject basis in FIGS. 68A-68G and FIGS. 69A-69G.

Subject 1, who lacks an ApoA-I gene (homozygote, ApoA-I−/−), showed cholesterol mobilization, LCAT activation, and fecal cholesterol elimination after one dose of CER-001 at 8 mg/Kg.

Subject 7, who has no ABCA1 gene (homozygote ABCA1−/−) and suffers from Tangier disease, showed cholesterol mobilization and LCAT activation after one dose of CER-001 at 8 mg/Kg. Fecal cholesterol elimination was not tested in this patient.

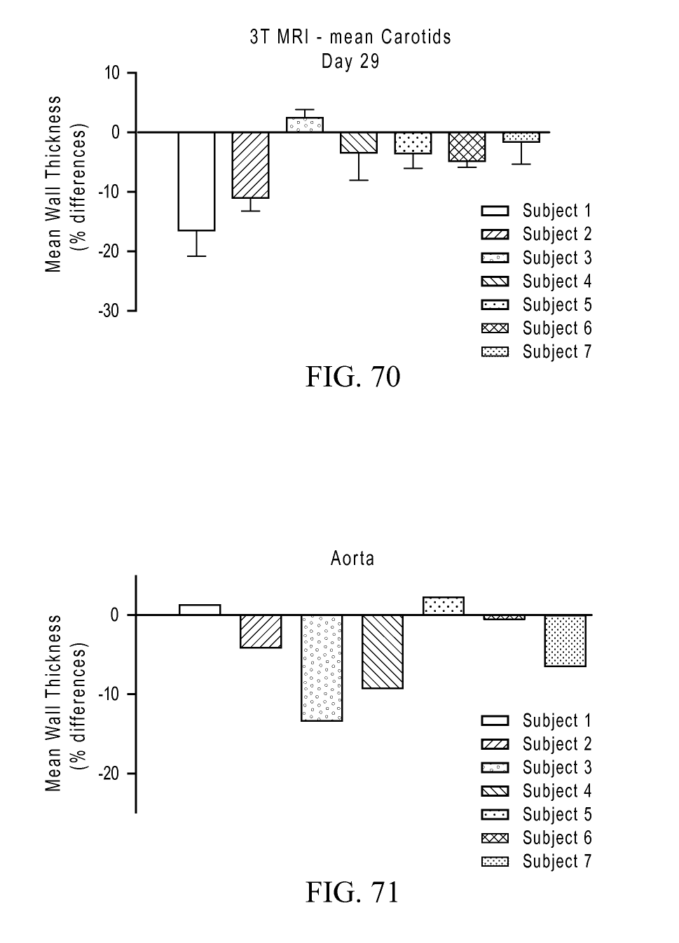

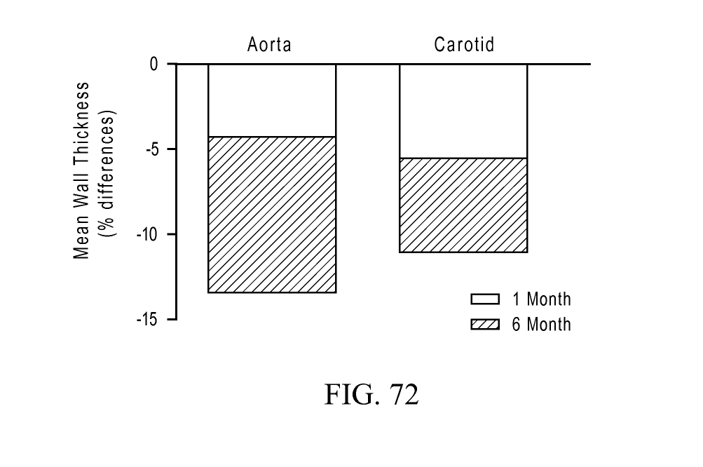

FIG. 70 and FIG. 71 show the mean carotic and aortic vessel wall thickness, respectively, on a patient-by-patient basis after one month of treatment. Mean vessel wall thickness of the carotid artery decreased by a mean of −6.4% after one month of induction therapy, and mean vessel wall thickness of the aorta decreased by a mean of −4.6% after one month of induction therapy. The homozygous ApoA-I deficiency patient experienced a −17% regression of carotid mean vessel wall thickness. FIG. 72 shows mean vessel wall thickness after 6 months. Mean vessel wall thickness was determined by 3 Tesla MRI.

This trial has demonstrated proof of mechanism (i.e., that CER-001 performs all the steps of the RLT pathway) as well as evidence of a positive therapeutic effect in these subjects, specifically a reduction in carotid intimal-medial wall thickness after one month of intensive treatment which, in the subject with the most profound defect (homozygous ABCA1 deficiency), was commensurate with the reductions seen after two years of treatment in statin-naïve hypercholesterolemic subjects. Importantly, the observed reductions were seen on top of standard of care (intensive individualized lipid management). Importantly, persistent and cumulative benefit was seen after an additional 5 months of maintenance therapy, supporting the therapeutic principle that patients with familial hypoalphalipoproteinemia require chronic ApoA-I replacement therapy for life. In ABCA1 deficiency, in absence of ABCA1 or in absence of ABCA1 function the cholesterol still effluxed to CER-001 probably because there is redundancy of the efflux pathway by other receptors such as but not limited to ABCG1.

### 11. SPECIFIC EMBODIMENTS

Various aspects of the present disclosure are described in the embodiments set forth in the following numbered paragraphs.

1. A method of identifying a dose of an HDL Therapeutic effective to mobilize cholesterol in a subject, comprising: (a) administering a first dose of an HDL Therapeutic to a subject, (b) following administering said first dose, measuring expression levels of one or more HDL Markers in said subject's circulating monocytes, macrophages or mononuclear cells to evaluate the effect of said first dose on said expression levels; and (c)(i) if the subject's expression levels of one or more HDL Markers are reduced by more than a cutoff amount, administering a second dose of said HDL Therapeutic, wherein the second dose of said HDL Therapeutic is lower than the first dose; or (ii) if the subject's expression levels of one or more HDL Markers are not reduced by more than the cutoff amount, treating the subject with the first dose of said HDL Therapeutic.

2. A method for monitoring the efficacy of an HDL Therapeutic in a subject, comprising: (a) treating a subject with an HDL Therapeutic according to a first dosing schedule, (b) measuring expression levels of one or more HDL Markers in said subject's circulating monocytes, macrophages or mononuclear cells to evaluate the effect of said first dosing schedule on said expression levels; and (c) (i) if the subject's expression levels of one or more HDL Markers are reduced by more than an upper cutoff amount, treating the subject with the HDL Therapeutic according to a second dosing schedule, wherein the second dosing schedule comprises one or more of: administering a lower dose of the HDL Therapeutic, infusing the HDL Therapeutic into the subject over a longer period of time, and administering the HDL Therapeutic to the subject on a less frequent basis; (ii) if the subject's expression levels of one or more HDL Markers are not reduced by more than a lower cutoff amount, treating the subject with the HDL Therapeutic according to a second dosing schedule, wherein the second dosing schedule comprises one or more of: administering a higher dose of the HDL Therapeutic, infusing the HDL Therapeutic into the subject over a shorter period of time, and administering the HDL Therapeutic to the subject on a more frequent basis; or (iii) if the subject's expression levels of one or more HDL Markers are reduced by an amount between the upper and lower cutoff amounts, continuing to treat the subject according to the first dosing schedule.

3. The method of embodiment 1 or embodiment 2, wherein the cutoff amount is relative to the subject's own baseline prior to said administration.

4. The method of embodiment 1 or embodiment 2, wherein the cutoff amount is relative to a control amount.

5. The method of embodiment 4, wherein the control amount is a population average.

6. The method of embodiment 5, wherein the population average is from healthy subjects.

7. The method of embodiment 5, wherein the population average is from a population with the same disease condition as the subject.

8. A method of identifying a dose of an HDL Therapeutic effective to mobilize cholesterol, comprising: (a) administering a first dose of an HDL Therapeutic to a population of subjects, (b) following administering said first dose, measuring expression levels of one or more HDL Markers in said subjects' circulating monocytes, macrophages or mononuclear cells to evaluate the effect of said first dose on said expression levels; (c) administering a second dose of said HDL Therapeutic, wherein the second dose of said HDL Therapeutic is greater or lower than the first dose, (d) following administering said second dose, measuring expression levels of one or more HDL Markers in said subjects' circulating monocytes, macrophages or mononuclear cells to evaluate the effect of said first and/or second dose on said expression levels; (e) optionally repeating steps (c) and (d) with one or more additional doses of said HDL Therapeutic; and (f) identifying the highest dose that does not reduce expression levels of one or more HDL Markers in by more than a cutoff amount, thereby identifying a dose of said HDL Therapeutic effective to mobilize cholesterol.

9. The method of embodiment 8, wherein step (d) comprises measuring expression levels of one or more HDL Markers in said subjects' circulating monocytes, macrophages or mononuclear cells following administering said second dose to evaluate the effect of said first dose on said expression levels.

10. The method of any one of embodiments 1 to 9, further comprising, following administering said second dose, measuring expression levels of one or more HDL Markers in said subject's circulating monocytes, macrophages or mononuclear cells to evaluate the effect of said second dose on said expression levels.

11. The method of embodiment 10, wherein if the subject's expression levels of one or more HDL Markers are reduced by more than a cutoff amount, administering a third dose of said HDL Therapeutic, wherein the third dose of said HDL Therapeutic is lower than the second dose.

12. A method for treating a subject in need of an HDL Therapeutic, comprising administering to subject a combination of: (a) an HDL Therapeutic, which is optionally a lipoprotein complex, in a dose that does not reduce expression of one or more HDL Markers in said subject's circulating monocytes, macrophages or mononuclear cells by more than 20% or more than 10% as compared to the subject's baseline amount; and (b) a cholesterol reducing therapy, optionally selected from a bile-acid resin, niacin, a statin, a fibrate, a PCSK9 inhibitor, ezetimibe, and a CETP inhibitor.

13. The method of embodiment 12, wherein the HDL Therapeutic is a lipoprotein complex.

14. A method for treating a subject in need of an HDL Therapeutic, comprising administering to subject a combination of: (a) an HDL Therapeutic, which is optionally a lipoprotein complex, in a dose that does not reduce expression of one or more HDL Markers in said subject's circulating monocytes, macrophages or mononuclear cells by more than 20% or more than 10% as compared to a control amount; and (b) a cholesterol reducing therapy, optionally selected from a bile-acid resin, niacin, a statin, a fibrate, a PCSK9 inhibitor, ezetimibe, and a CETP inhibitor.

15. The method of embodiment 14, wherein the HDL Therapeutic is a lipoprotein complex.

16. The method of embodiment 14 or 15, wherein the control amount is a population average.

17. The method of embodiment 16, wherein the population average is from healthy subjects.

18. The method of embodiment 16, wherein the population average is from a population with the same disease condition as the subject.

19. The method of any one of embodiments 1 to 18, wherein the subject is human or the population of subjects is a population of human subjects.

20. The method of any one of embodiments 1 to 18, wherein the subject is a non-human animal or the population of subjects is a population of non-human animals.

21. The method of embodiment 20, wherein the non-human animal is a mouse.

22. The method of any one of embodiments 1 to 21, wherein at least one HDL Marker is ABCA1.

23. The method of embodiment 22, wherein ABCA1 mRNA expression levels are measured.

24. The method of embodiment 22, wherein ABCA1 protein expression levels are measured.

25. The method of any one of embodiments 22 to 24, wherein the ABCA1 cutoff amount is 20%-80%.

26. The method of embodiment 25, wherein the ABCA1 cutoff amount is 30%-70%.

27. The method of embodiment 26, wherein the ABCA1 cutoff amount is 40%-60%.

28. The method of embodiment 27, wherein the ABCA1 cutoff amount is 50%.

29. The method of any one of embodiments 22 to 28, wherein ABCA1 expression levels are measured 2-12 hours, 4-10 hours, 2-8 hours, 2-6 hours, 4-6 hours or 4-8 hours after administration of said first dose or said second dose.

30. The method of any one of embodiments 1 to 29, wherein at least one HDL Marker is ABCG1.

31. The method of embodiment 30, wherein ABCG1 mRNA expression levels are measured.

32. The method of embodiment 30, wherein ABCG1 protein expression levels are measured.

33. The method of any one of embodiments 30 to 32, wherein the ABCG1 cutoff amount is 20%-80%.

34. The method of embodiment 33, wherein the ABCG1 cutoff amount is 30%-70%.

35. The method of embodiment 34, wherein the ABCG1 cutoff amount is 40%-60%.

36. The method of embodiment 35, wherein the ABCA1 cutoff amount is 50%.

37. The method of any one of embodiments 30 to 36, wherein ABCG1 expression levels are measured 2-12 hours, 4-10 hours, 2-8 hours, 2-6 hours, 4-6 hours or 4-8 hours after administration.

38. The method of any one of embodiments 1 to 37, wherein at least one HDL Marker is SREBP-1.

39. The method of embodiment 38, wherein SREBP-1 mRNA expression levels are measured.

40. The method of embodiment 38, wherein SREBP-1 protein expression levels are measured.

41. The method of any one of embodiments 38 to 40, wherein the SREBP-1 cutoff amount is 20%-80%.

42. The method of embodiment 41, wherein the SREBP-1 cutoff amount is 30%-70%.

43. The method of embodiment 42, wherein the SREBP-1 cutoff amount is 40%-60%.

44. The method of embodiment 43, wherein the SREBP-1 cutoff amount is 50%.

45. The method of any one of embodiments 38 to 44, wherein SREBP-1 expression levels are measured 2-12 hours, 4-10 hours, 2-8 hours, 2-6 hours, 4-6 hours or 4-8 hours after administration.

46. The method of any one of embodiments 1 to 45, wherein the HDL Therapeutic is a lipoprotein complex.

47. The method of embodiment 46, wherein the lipoprotein complex comprises an apolipoprotein.

48. The method of embodiment 47, wherein the apolipoprotein is ApoA-I, ApoA-II, ApoA-IV, ApoE or a combination thereof.

49. The method of embodiment 46, wherein the lipoprotein complex comprises an apolipoprotein peptide mimic.

50. The method of embodiment 49, wherein the peptide mimic is an ApoA-I, ApoA-II, ApoA-IV, or ApoE peptide mimic or a combination thereof.

51. The method of embodiment 46, wherein the lipoprotein complex is CER-001, CSL-111, CSL-112, or ETC-216.

52. The method of any one of embodiments 1 to 45, wherein the HDL Therapeutic is a small molecule.

53. The method of embodiment 52, wherein the small molecule is a CETP inhibitor.

54. The method of embodiment 52, wherein the small molecule is a pantothenic acid derivatives.

55. The method of any one of embodiments 1 to 46 which further comprises determining a cutoff amount.

56. The method of embodiment 55, wherein the cutoff amount is determined by generating a dose response curve for the HDL Therapeutic.

57. The method of embodiment 56, wherein the cutoff amount is 25%-75% of the dose that results in an inflection point in the dose response curve.

58. The method of embodiment 57, wherein the cutoff amount is 40%-60% of the dose that results in an inflection point in the dose response curve.

59. The method of any one of embodiments 1 to 58, wherein the subject or population of subjects has an ABCA1 deficiency.

60. The method of embodiment 59, wherein the subject or population of subjects is homozygous for an ABCA1 mutation.

61. The method of embodiment 59, wherein the subject or population of subjects is heterozygous for an ABCA1 mutation.

62. A method of identifying a dose of an HDL Therapeutic suitable for therapy, comprising: (a) administering one or more doses of an HDL Therapeutic to a subject, (b) measuring expression levels of one or more HDL Markers in said subject's circulating monocytes, macrophages or mononuclear cells following each dose; and (c) identifying the maximum dose that does not raise expression levels of said one or more HDL Markers by more than 0%, more than 10% or more than 20%, thereby identifying a dose of an HDL Therapeutic suitable for therapy.

63. A method of identifying a dose of an HDL Therapeutic suitable for therapy, comprising: (a) administering one or more doses of an HDL Therapeutic to a population of subjects, (b) measuring expression levels of one or more HDL Markers in said subjects' circulating monocytes, macrophages or mononuclear cells following each dose; and (c) identifying the maximum dose that does not raise expression levels of said one or more HDL Markers by more than 0%, more than 10% or more than 20% in said subjects, thereby identifying a dose of an HDL Therapeutic suitable for therapy.

64. A method of identifying a dose of an HDL Therapeutic suitable for therapy, comprising identifying the highest dose of the HDL therapeutic that does not reduce cellular cholesterol efflux by more than 0%, more than 10% or more than 20%.

65. The method of embodiment 64, which comprises: (a) administering one or more doses of an HDL Therapeutic to a subject or population of subjects; (b) measuring cholesterol efflux in cells from said subject or population of subjects; and (c) identifying the maximum dose that does not reduce cholesterol efflux by more than 0%, more than 10% or more than 20% in said subjects, thereby identifying a dose of an HDL Therapeutic suitable for therapy.

66. A method of identifying a dosing interval of an HDL Therapeutic suitable for therapy, comprising identifying the highest dose of the most frequent dosing regimen of the HDL therapeutic that does not reduce cellular cholesterol efflux by more than 0%, more than 10% or more than 20%.

67. The method of embodiment 66, which comprises: (a) administering an HDL Therapeutic to a subject or population of subjects according to one or more dosing frequencies; (b) measuring cholesterol efflux in cells from said subject or population of subjects; and (c) identifying the maximum dosing frequency that does not reduce cholesterol efflux by more than 0%, more than 10% or more than 20% in said subjects, thereby identifying a dose of an HDL Therapeutic suitable for therapy.

68. The method of embodiment 67, wherein the one or more dosing frequencies includes one or more dosing frequencies selected from: (a) administration as a 1-4 hour infusion every 2 days; (b) administration as a 1-4 hour an infusion every 3 days; (c) administration as a 24 hour infusion every week days; and (d) administration as a 24 hour an infusion every two weeks.

69. The method of any one of embodiments 65 to 68, wherein cholesterol efflux is measured in monocytes, macrophages or mononuclear cells from said subjects or populations of subjects.

70. A method for treating a subject with an ABCA1 deficiency, comprising administering to the subject a therapeutically effective amount of an HDL Therapeutic.

71. The method of embodiment 70, wherein the HDL Therapeutic is CER-001.

72. The method of embodiment 70 or 71, wherein the subject is heterozygous for an ABCA1 mutation.

73. The method of embodiment 70 or 71, wherein the subject is homozygous for an ABCA1 mutation.

74. A method of treating a subject suffering from familial primary hypoalphalipoproteinemia, comprising: (a) administering to the subject an HDL Therapeutic according to an induction regimen; and, subsequently (b) administering to the subject the HDL Therapeutic according to a maintenance regimen.

75. The method of embodiment 74, wherein the maintenance regimen entails administering the HDL therapeutic at a lower dose, a lower frequency, or both.

76. The method of embodiment 74 or embodiment 75, wherein the subject is heterozygous for an ABCA1 mutation.

77. The method of embodiment 74 or embodiment 75, wherein the subject is homozygous for an ABCA1 mutation.

78. The method of any one of embodiments 74 to 77, wherein the subject is homozygous or heterozygous for an LCAT mutation.

79. The method of any one of embodiments 74 to 78, wherein the subject is homozygous or heterozygous for an ApoA-I mutation.

80. The method of any one of embodiments 74 to 79, wherein the subject is homozygous or heterozygous for an ABCG1 mutation.

81. The method of any one of embodiments 74 to 80, wherein the subject is also treated with a lipid control medication.

82. The method of embodiment 81, wherein the lipid control medication is atorvastatin, ezetimibe, niacin, rosuvastatin, simvastatin, aspirin, fluvastatin, lovastatin, pravastatin or a combination thereof.

83. The method of any one of embodiments 74 to 82, wherein the HDL Therapeutic is CER-001.

84. The method of embodiment 83, wherein the induction regimen is of a duration of 4 weeks.

85. The method of embodiment 83 or embodiment 84, wherein the induction regimen comprises administering CER-001 three times a week.

86. The method of any one of embodiments 83 to 85, wherein the dose administered in the induction regimen is 8-15 mg/kg (on a protein weight basis).

87. The method of embodiment to 86, wherein the dose administered in the induction regimen is 8 mg/kg, 12 mg/kg or 15 mg/kg.

88. The method of any one of embodiments 83 to 87, wherein the maintenance regimen comprises administering CER-001 for at least one month, at least two months, at least three months, at least six months, at least a year, at least 18 months, at least two years, or indefinitely.

89. The method of any one of embodiments 83 to 88, wherein the maintenance regimen comprises administering CER-001 twice a week.

90. The method of any one of embodiments 83 to 89, wherein the dose administered in the maintenance regimen is 1-6 mg/kg (on a protein weight basis).

91. The method of embodiment 90, wherein the dose administered in the maintenance regimen is 1 mg/kg, 3 mg/kg or 6 mg/kg.

92. The method of any one of embodiments 74 to 91, wherein: (a) the induction regimen utilizes a dose that reduces expression levels of one or more HDL Markers by 20%-80% or 40%-60%, as compared to the subject's baseline amount and/or a population average; and/or (b) the maintenance regimen utilizes a dose that does not reduce expression levels of one or more HDL Markers by more than 20% or more than 10% as compared to the subject's baseline amount and/or a population average.

93. The method of embodiment 92, wherein the maintenance regimen utilizes a dose that does not reduce expression levels of one or more HDL Markers.

94. A HDL Therapeutic for use in a method of identifying a dose of the HDL Therapeutic effective to mobilize cholesterol in a subject, the method comprising: (a) administering a first dose of the HDL Therapeutic to a subject, (b) following administering said first dose, measuring expression levels of one or more HDL Markers in said subject's circulating monocytes, macrophages or mononuclear cells to evaluate the effect of said first dose on said expression levels; and (c) (i) if the subject's expression levels of one or more HDL Markers are reduced by more than a cutoff amount, administering a second dose of said HDL Therapeutic, wherein the second dose of said HDL Therapeutic is lower than the first dose; or (ii) if the subject's expression levels of one or more HDL Markers are not reduced by more than the cutoff amount, treating the subject with the first dose of said HDL Therapeutic.

95. A HDL Therapeutic for use in a method for monitoring the efficacy of the HDL Therapeutic in a subject, the method comprising: (a) treating a subject with the HDL Therapeutic according to a first dosing schedule, (b) measuring expression levels of one or more HDL Markers in said subject's circulating monocytes, macrophages or mononuclear cells to evaluate the effect of said first dosing schedule on said expression levels; and (c)(i) if the subject's expression levels of one or more HDL Markers are reduced by more than an upper cutoff amount, treating the subject with the HDL Therapeutic according to a second dosing schedule, wherein the second dosing schedule comprises one or more of: administering a lower dose of the HDL Therapeutic, infusing the HDL Therapeutic into the subject over a longer period of time, and administering the HDL Therapeutic to the subject on a less frequent basis; (ii) if the subject's expression levels of one or more HDL Markers are not reduced by more than a lower cutoff amount, treating the subject with the HDL Therapeutic according to a second dosing schedule, wherein the second dosing schedule comprises one or more of: administering a higher dose of the HDL Therapeutic, infusing the HDL Therapeutic into the subject over a shorter period of time, and administering the HDL Therapeutic to the subject on a more frequent basis; or (iii) if the subject's expression levels of one or more HDL Markers are reduced by an amount between the upper and lower cutoff amounts, continuing to treat the subject according to the first dosing schedule.

96. The HDL Therapeutic for use of embodiment 94 or embodiment 95, wherein the cutoff amount is relative to the subject's own baseline prior to said administration.

97. The HDL Therapeutic for use of embodiment 94 or embodiment 95, wherein the cutoff amount is relative to a control amount.

98. The HDL Therapeutic for use of embodiment 97, wherein the control amount is a population average.

99. The HDL Therapeutic for use of embodiment 98, wherein the population average is from healthy subjects.

100. The HDL Therapeutic for use of embodiment 98, wherein the population average is from a population with the same disease condition as the subject.

101. A HDL Therapeutic for use in a method of identifying a dose of an HDL Therapeutic effective to mobilize cholesterol, the method comprising: (a) administering a first dose of an HDL Therapeutic to a population of subjects, (b) following administering said first dose, measuring expression levels of one or more HDL Markers in said subjects' circulating monocytes, macrophages or mononuclear cells to evaluate the effect of said first dose on said expression levels; (c) administering a second dose of said HDL Therapeutic, wherein the second dose of said HDL Therapeutic is greater or lower than the first dose, (d) following administering said second dose, measuring expression levels of one or more HDL Markers in said subjects' circulating monocytes, macrophages or mononuclear cells to evaluate the effect of said first and/or second dose on said expression levels; (e) optionally repeating steps (c) and (d) with one or more additional doses of said HDL Therapeutic; and (f) identifying the highest dose that does not reduce expression levels of one or more HDL Markers in by more than a cutoff amount, thereby identifying a dose of said HDL Therapeutic effective to mobilize cholesterol.

102. The method of embodiment 101, wherein step (d) comprises measuring expression levels of one or more HDL Markers in said subjects' circulating monocytes, macrophages or mononuclear cells following administering said second dose to evaluate the effect of said first dose on said expression levels.

103. The HDL Therapeutic for use of any one of embodiments 94 to 101, the method further comprising, following administering said second dose, measuring expression levels of one or more HDL Markers in said subject's circulating monocytes, macrophages or mononuclear cells to evaluate the effect of said second dose on said expression levels.

104. The HDL Therapeutic for use of embodiment 102, wherein if the subject's expression levels of one or more HDL Markers are reduced by more than a cutoff amount, a third dose of said HDL Therapeutic is administered, wherein the third dose of said HDL Therapeutic is lower than the second dose.

105. A HDL Therapeutic, which is optionally a lipoprotein complex, for use in a method for treating a subject in need of an HDL Therapeutic, the method comprising administering to the subject a combination of: (a) the HDL Therapeutic in a dose that does not reduce expression of one or more HDL Markers in said subject's circulating monocytes, macrophages or mononuclear cells by more than 20% or more than 10% as compared to the subject's baseline amount or to a control amount; and (b) a cholesterol reducing therapy, optionally selected from a bile-acid resin, niacin, a statin, a fibrate, a PCSK9 inhibitor, ezetimibe, and a CETP inhibitor.

106. The HDL Therapeutic for use of embodiment 105, which is a lipoprotein complex.

107. The HDL Therapeutic for use of embodiment 105 or 106, wherein the compared amount is the subject's baseline amount.

108. The HDL Therapeutic for use of embodiment 105 or 106, wherein the compared amount is a control amount and is a population average.

109. The HDL Therapeutic for use of embodiment 108, wherein the population average is from healthy subjects.

110. The HDL Therapeutic for use of embodiment 108, wherein the population average is from a population with the same disease condition as the subject.

111. The HDL Therapeutic for use of any one of embodiments 94 to 110, wherein the subject is human or the population of subjects is a population of human subjects.

112. The HDL Therapeutic for use of any one of embodiments 94 to 110, wherein the subject is a non-human animal or the population of subjects is a population of non-human animals.

113. The HDL Therapeutic for use of embodiment 112, wherein the non-human animal is a mouse.

114. The HDL Therapeutic for use of any one of embodiments 94 to 113, wherein at least one HDL Marker is ABCA1.

115. The HDL Therapeutic for use of embodiment 114, wherein ABCA1 mRNA expression levels are measured.

116. The HDL Therapeutic for use of embodiment 114, wherein ABCA1 protein expression levels are measured.

117. The HDL Therapeutic for use of any one of embodiments 114 to 116, wherein the ABCA1 cutoff amount is 20%-80%.

118. The HDL Therapeutic for use of embodiment 117, wherein the ABCA1 cutoff amount is 30%-70%.

119. The HDL Therapeutic for use of embodiment 118, wherein the ABCA1 cutoff amount is 40%-60%.

120. The HDL Therapeutic for use of embodiment 119, wherein the ABCA1 cutoff amount is 50%.

121. The HDL Therapeutic for use of any one of embodiments 114 to 120, wherein ABCA1 expression levels are measured 2-12 hours, 4-10 hours, 2-8 hours, 2-6 hours, 4-6 hours or 4-8 hours after administration of said first dose or said second dose.

122. The HDL Therapeutic for use of any one of embodiments 94 to 121, wherein at least one HDL Marker is ABCG1.

123. The HDL Therapeutic for use of embodiment 122, wherein ABCG1 mRNA expression levels are measured.

124. The HDL Therapeutic for use of embodiment 122, wherein ABCG1 protein expression levels are measured.

125. The HDL Therapeutic for use of any one of embodiments 122 to 124, wherein the ABCG1 cutoff amount is 20%-80%.

126. The HDL Therapeutic for use of embodiment 125, wherein the ABCG1 cutoff amount is 30%-70%.

127. The HDL Therapeutic for use of embodiment 126, wherein the ABCG1 cutoff amount is 40%-60%.

128. The HDL Therapeutic for use of embodiment 127, wherein the ABCA1 cutoff amount is 50%.

129. The HDL Therapeutic for use of any one of embodiments 122 to 128, wherein ABCG1 expression levels are measured 2-12 hours, 4-10 hours, 2-8 hours, 2-6 hours, 4-6 hours or 4-8 hours after administration.

130. The HDL Therapeutic for use of any one of embodiments 94 to 129, wherein at least one HDL Marker is SREBP-1.

131. The HDL Therapeutic for use of embodiment 130, wherein SREBP-1 mRNA expression levels are measured.

132. The HDL Therapeutic for use of embodiment 130, wherein SREBP-1 protein expression levels are measured.

133. The HDL Therapeutic for use of any one of embodiments 130 to 132, wherein the SREBP-1 cutoff amount is 20%-80%.

134. The HDL Therapeutic for use of embodiment 133, wherein the SREBP-1 cutoff amount is 30%-70%.

135. The HDL Therapeutic for use of embodiment 134, wherein the SREBP-1 cutoff amount is 40%-60%.

136. The HDL Therapeutic for use of embodiment 135, wherein the SREBP-1 cutoff amount is 50%.

137. The HDL Therapeutic for use of any one of embodiments 130 to 136, wherein SREBP-1 expression levels are measured 2-12 hours, 4-10 hours, 2-8 hours, 2-6 hours, 4-6 hours or 4-8 hours after administration.

138. The HDL Therapeutic for use of any one of embodiments 94 to 137, wherein the HDL Therapeutic is a lipoprotein complex.

139. The HDL Therapeutic for use of embodiment 138, wherein the lipoprotein complex comprises an apolipoprotein.

140. The HDL Therapeutic for use of embodiment 139, wherein the apolipoprotein is ApoA-I, ApoA-II, ApoA-IV, ApoE or a combination thereof.

141. The HDL Therapeutic for use of embodiment 138, wherein the lipoprotein complex comprises an apolipoprotein peptide mimic.

142. The HDL Therapeutic for use of embodiment 141, wherein the peptide mimic is an ApoA-I, ApoA-II, ApoA-IV, or ApoE peptide mimic or a combination thereof.

143. The HDL Therapeutic for use of embodiment 138, wherein the lipoprotein complex is CER-001, CSL-111, CSL-112, or ETC-216.

144. The HDL Therapeutic for use of any one of embodiments 94 to 137, wherein the HDL Therapeutic is a small molecule.

145. The HDL Therapeutic for use of embodiment 144, wherein the small molecule is a CETP inhibitor.

146. The HDL Therapeutic for use of embodiment 144, wherein the small molecule is a pantothenic acid derivative.

147. The HDL Therapeutic for use of any one of embodiments 94 to 138 which further comprises determining a cutoff amount.

148. The HDL Therapeutic for use of embodiment 147, wherein the cutoff amount is determined by generating a dose response curve for the HDL Therapeutic.

149. The HDL Therapeutic for use of embodiment 148, wherein the cutoff amount is 25%-75% of the dose that results in an inflection point in the dose response curve.

150. The HDL Therapeutic for use of embodiment 149, wherein the cutoff amount is 40%-60% of the dose that results in an inflection point in the dose response curve.

151. The HDL Therapeutic for use of any one of embodiments 94 to 150, wherein the subject or population of subjects has an ABCA1 deficiency.

152. The HDL Therapeutic for use of embodiment 151, wherein the subject or population of subjects is homozygous for an ABCA1 mutation.

153. The HDL Therapeutic for use of embodiment 151, wherein the subject or population of subjects is heterozygous for an ABCA1 mutation.

154. A HDL Therapeutic for use in a method of identifying a dose of the HDL Therapeutic suitable for therapy, the method comprising: (a) administering one or more doses of the HDL Therapeutic to a subject, (b) measuring expression levels of one or more HDL Markers in said subject's circulating monocytes, macrophages or mononuclear cells following each dose; and (c) identifying the maximum dose that does not raise expression levels of said one or more HDL Markers by more than 0%, more than 10% or more than 20%, thereby identifying a dose of an HDL Therapeutic suitable for therapy.

155. A HDL Therapeutic for use in a method of identifying a dose of the HDL Therapeutic suitable for therapy, the method comprising: (a) administering one or more doses of the HDL Therapeutic to a population of subjects, (b) measuring expression levels of one or more HDL Markers in said subjects' circulating monocytes, macrophages or mononuclear cells following each dose; and (c) identifying the maximum dose that does not raise expression levels of said one or more HDL Markers by more than 0%, more than 10% or more than 20% in said subjects, thereby identifying a dose of an HDL Therapeutic suitable for therapy.

156. A HDL Therapeutic for use in a method of identifying a dose of the HDL Therapeutic suitable for therapy, comprising identifying the highest dose of the HDL therapeutic that does not reduce cellular cholesterol efflux by more than 0%, more than 10% or more than 20%.

157. The HDL Therapeutic for use of embodiment 156, which comprises: (a) administering an HDL Therapeutic to a subject or population of subjects according to one or more dosing frequencies; (b) measuring cholesterol efflux in cells from said subject or population of subjects; and (c) identifying the maximum dosing frequency that does not reduce cholesterol efflux by more than 50% to 100% in said subjects, thereby identifying a dose of an HDL Therapeutic suitable for therapy.

158. A HDL Therapeutic for use in a method of identifying a dosing interval of an HDL Therapeutic suitable for therapy, comprising identifying the highest dose of the most frequent dosing regimen of the HDL therapeutic that does not reduce cellular cholesterol efflux by more than 0%, more than 10% or more than 20%.

159. The HDL Therapeutic for use of embodiment 158, which comprises: (a) administering an HDL Therapeutic to a subject or population of subjects according to one or more dosing frequencies; (b) measuring cholesterol efflux in cells from said subject or population of subjects; and (c) identifying the maximum dosing frequency that does not reduce cholesterol efflux by more than 50% to 100% in said subjects, thereby identifying a dose of an HDL Therapeutic suitable for therapy.

160. A HDL Therapeutic for use in a method of identifying a dose of an HDL Therapeutic suitable for therapy, the method comprising (a) administering one or more doses of an HDL Therapeutic to a subject or population of subjects; (b) measuring cholesterol efflux in cells from said subject or population of subjects; and (c) identifying the maximum dose that does not reduce cholesterol efflux by more than 0%, more than 10% or more than 20% in said subjects, thereby identifying a dose of an HDL Therapeutic suitable for therapy.

161. A HDL Therapeutic for use in a method of identifying a dosing interval of an HDL Therapeutic suitable for therapy, the method comprising identifying the highest dose of the most frequent dosing regimen of the HDL therapeutic by the steps of (a) administering an HDL Therapeutic to a subject or population of subjects according to one or more dosing frequencies; (b) measuring cholesterol efflux in cells from said subject or population of subjects; and (c) identifying the maximum dosing frequency that does not reduce cholesterol efflux by more than 0%, more than 10% or more than 20% in said subjects, thereby identifying a dose of an HDL Therapeutic suitable for therapy.

162. The HDL Therapeutic for use of embodiment 161, wherein the one or more dosing frequencies includes one or more dosing frequencies selected from: (a) administration as a 1-4 hour infusion every 2 days; (b) administration as a 1-4 hour an infusion every 3 days; (c) administration as a 24 hour infusion every week days; and (d) administration as a 24 hour an infusion every two weeks.

163. The HDL Therapeutic for use of any one of embodiments 156 to 162, wherein cholesterol efflux is measured in monocytes, macrophages or mononuclear cells from said subjects or populations of subjects.

164. A HDL Therapeutic for use in a method for treating a subject with an ABCA1 deficiency, comprising administering to the subject a therapeutically effective amount the HDL Therapeutic.

165. The HDL Therapeutic for use of embodiment 164, wherein the HDL Therapeutic is CER-001.

166. The HDL Therapeutic for use of embodiments 164 or 165, wherein the subject is heterozygous for an ABCA1 mutation.

167. The HDL Therapeutic for use of embodiments 164 or 165, wherein the subject is homozygous for an ABCA1 mutation.

168. A HDL Therapeutic for use in a method of treating a subject suffering from familial primary hypoalphalipoproteinemia, the method comprising: (a) administering to the subject the HDL Therapeutic according to an induction regimen; and, subsequently (b) administering to the subject the HDL Therapeutic according to a maintenance regimen.

169. The HDL Therapeutic for use of embodiment 168, wherein the maintenance regimen entails administering the HDL therapeutic at a lower dose, a lower frequency, or both.

170. The HDL Therapeutic for use of embodiment 168 or embodiment 169, wherein the subject is heterozygous for an ABCA1 mutation.

171. The HDL Therapeutic for use of embodiment 168 or embodiment 169, wherein the subject is homozygous for an ABCA1 mutation.

172. The HDL Therapeutic for use of any one of embodiments 168 to 171, wherein the subject is homozygous or heterozygous for an LCAT mutation.

173. The HDL Therapeutic for use of any one of embodiments 168 to 172, wherein the subject is homozygous or heterozygous for an ApoA-I mutation.

174. The HDL Therapeutic for use of any one of embodiments 168 to 173, wherein the subject is homozygous or heterozygous for an ABCG1 mutation.

175. The HDL Therapeutic for use of any one of embodiments 168 to 174, wherein the subject is also treated with a lipid control medication.

176. The HDL Therapeutic for use of embodiment 175, wherein the lipid control medication is atorvastatin, ezetimibe, niacin, rosuvastatin, simvastatin, aspirin, fluvastatin, lovastatin, pravastatin or a combination thereof.

177. The HDL Therapeutic for use of any one of embodiments 168 to 176, wherein the HDL Therapeutic is CER-001.

178. The HDL Therapeutic for use of embodiment 177, wherein the induction regimen is of a duration of 4 weeks.

179. The HDL Therapeutic for use of embodiment 177 or embodiment 178, wherein the induction regimen comprises administering CER-001 three times a week.

180. The HDL Therapeutic for use of any one of embodiments 177 to 179, wherein the dose administered in the induction regimen is 8-15 mg/kg (on a protein weight basis).

181. The HDL Therapeutic for use of embodiment to 180, wherein the dose administered in the induction regimen is 8 mg/kg, 12 mg/kg or 15 mg/kg.

182. The HDL Therapeutic for use of any one of embodiments 177 to 181, wherein the maintenance regimen comprises administering CER-001 for at least one month, at least two months, at least three months, at least six months, at least a year, at least 18 months, at least two years, or indefinitely.

183. The HDL Therapeutic for use of any one of embodiments 177 to 182, wherein the maintenance regimen comprises administering CER-001 twice a week.

184. The HDL Therapeutic for use of any one of embodiments 177 to 183, wherein the dose administered in the maintenance regimen is 1-6 mg/kg (on a protein weight basis).

185. The HDL Therapeutic for use of embodiment 184, wherein the dose administered in the maintenance regimen is 1 mg/kg, 3 mg/kg or 6 mg/kg.

186. The HDL Therapeutic for use of any one of embodiments 168 to 185, wherein: (a) the induction regimen utilizes a dose that reduces expression levels of one or more HDL Markers by 20%-80% or 40%-60%, as compared to the subject's baseline amount and/or a population average; and/or (b) the maintenance regimen utilizes a dose that does not reduce expression levels of one or more HDL Markers by more than 20% or more than 10% as compared to the subject's baseline amount and/or a population average.

187. The HDL Therapeutic for use of embodiment 186, wherein the maintenance regimen utilizes a dose that does not reduce expression levels of one or more HDL Markers.

While various specific embodiments have been illustrated and described, it will be appreciated that various changes can be made without departing from the spirit and scope of the disclosure(s).

### 12. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

Any discussion of documents, acts, materials, devices, articles or the like that has been included in this specification is solely for the purpose of providing a context for the present disclosure. It is not to be taken as an admission that any or all of these matters form part of the prior art base or were common general knowledge in the field relevant to the present disclosure as it existed anywhere before the priority date of this application.

What is claimed is:

1. A method of identifying a dose of a HDL Therapeutic effective to mobilize cholesterol in a subject, comprising:
(a) administering a first dose of a HDL Therapeutic to a subject,
(b) following administering said first dose, measuring expression levels of one or more HDL Markers in said subject's circulating monocytes, macrophages or mononuclear cells to evaluate the effect of said first dose on said expression levels; and
(c) (i) if the subject's expression levels of one or more HDL Markers are reduced by more than a cutoff amount, administering a second dose of said HDL Therapeutic, wherein the second dose of said HDL Therapeutic is lower than the first dose; or
(ii) if the subject's expression levels of one or more HDL Markers are not reduced by more than the cutoff amount, treating the subject with the first dose of said HDL Therapeutic.

2. A method for monitoring the efficacy of a HDL Therapeutic in a subject, comprising:
(a) treating a subject with a HDL Therapeutic according to a first dosing schedule,
(b) measuring expression levels of one or more HDL Markers in said subject's circulating monocytes, macrophages or mononuclear cells to evaluate the effect of said first dosing schedule on said expression levels; and
(c) (i) if the subject's expression levels of one or more HDL Markers are reduced by more than an upper cutoff amount, treating the subject with the HDL Therapeutic according to a second dosing schedule, wherein the second dosing schedule comprises one or more of: administering a lower dose of the HDL Therapeutic, infusing the HDL Therapeutic into the subject over a longer period of time, and administering the HDL Therapeutic to the subject on a less frequent basis;
(ii) if the subject's expression levels of one or more HDL Markers are not reduced by more than a lower cutoff amount, treating the subject with the HDL Therapeutic according to a second dosing schedule, wherein the second dosing schedule comprises one or more of: administering a higher dose of the HDL Therapeutic, infusing the HDL Therapeutic into the subject over a shorter period of time, and administering the HDL Therapeutic to the subject on a more frequent basis; or
(iii) if the subject's expression levels of one or more HDL Markers are reduced by an amount between the upper and lower cutoff amounts, continuing to treat the subject according to the first dosing schedule.

3. The method of claim 1, wherein the cutoff amount is relative to the subject's own baseline prior to said administration or relative to a control amount.

4. The method of claim 3, wherein the control amount is a population average, optionally from healthy subjects or from a population with the same disease condition as the subject.

5. A method of identifying a dose of a HDL Therapeutic effective to mobilize cholesterol, comprising:
(a) administering a first dose of a HDL Therapeutic to a population of subjects,
(b) following administering said first dose, measuring expression levels of one or more HDL Markers in said subjects' circulating monocytes, macrophages or mononuclear cells to evaluate the effect of said first dose on said expression levels;
(c) administering a second dose of said HDL Therapeutic, wherein the second dose of said HDL Therapeutic is greater or lower than the first dose,
(d) following administering said second dose, measuring expression levels of one or more HDL Markers in said subjects' circulating monocytes, macrophages or mononuclear cells to evaluate the effect of said first and/or second dose on said expression levels;
(e) optionally repeating steps (c) and (d) with one or more additional doses of said HDL Therapeutic; and
(f) identifying the highest dose that does not reduce expression levels of one or more HDL Markers in by more than a cutoff amount, thereby identifying a dose of said HDL Therapeutic effective to mobilize cholesterol.

6. The method of claim 5, wherein step (d) comprises measuring expression levels of one or more HDL Markers in said subjects' circulating monocytes, macrophages or mononuclear cells following administering said second dose to evaluate the effect of said first dose on said expression levels.

7. The method of claim 1, further comprising:
(a) following administering said second dose, measuring expression levels of one or more HDL Markers in said subject's circulating monocytes, macrophages or mononuclear cells to evaluate the effect of said second dose on said expression levels; and
(b) optionally, if the subject's expression levels of one or more HDL Markers are reduced by more than a cutoff amount, administering a third dose of said HDL Therapeutic, wherein the third dose of said HDL Therapeutic is lower than the second dose.

8. A method for treating a subject in need of an HDL Therapeutic, comprising administering to subject a combination of:
I. (a) an HDL Therapeutic, which is optionally a lipoprotein complex, in a dose that does not reduce expression of one or more HDL Markers in said subject's circulating monocytes, macrophages or mononuclear cells by more than 20% or more than 10% as compared to the subject's baseline amount; and
(b) a cholesterol reducing therapy, optionally selected from a bile-acid resin, niacin, a statin, a fibrate, a PCSK9 inhibitor, ezetimibe, and a CETP inhibitor; or
II. (a) an HDL Therapeutic, which is optionally a lipoprotein complex, in a dose that does not reduce expression of one or more HDL Markers in said subject's circulating monocytes, macrophages or mononuclear cells by more than 20% or more than 10% as compared to a control amount; and
(b) a cholesterol reducing therapy, optionally selected from a bile-acid resin, niacin, a statin, a fibrate, a PCSK9 inhibitor, ezetimibe, and a CETP inhibitor.

9. The method of claim 8, wherein the control amount is a population average, optionally from healthy subjects or from a population with the same disease condition as the subject.

10. The method of claim 1, wherein the subject is human or wherein the subject is a non-human animal.

11. The method of claim 10, wherein the non-human animal is a mouse.

12. The method of claim 1, wherein at least one HDL Marker is ABCA1.

13. The method of claim 12, wherein
(a) ABCA1 mRNA expression levels are measured or ABCA1 protein expression levels are measured;
(b) the ABCA1 cutoff amount is 20%-80%, 30%-70%, 40%-60%, or 50%; and/or
(c) ABCA1 expression levels are measured 2-12 hours, 4-10 hours, 2-8 hours, 2-6 hours, 4-6 hours or 4-8 hours after administration of said first dose or said second dose.

14. The method of claim 1, wherein at least one HDL Marker is ABCG1, and optionally wherein:
(a) ABCG1 mRNA expression levels are measured or ABCG1 protein expression levels are measured;
(b) the ABCG1 cutoff amount is 20%-80%, 30%-70%, 40%-60%, or 50%; and/or
(c) ABCG1 expression levels are measured 2-12 hours, 4-10 hours, 2-8 hours, 2-6 hours, 4-6 hours or 4-8 hours after administration.

15. The method of claim 1, wherein at least one HDL Marker is SREBP-1, and optionally wherein:
(a) SREBP-1 mRNA expression levels are measured or SREBP-1 protein expression levels are measured;
(b) the SREBP-1 cutoff amount is 20%-80%, 30%-70%, 40%-60%, or 50%; and/or
(c) SREBP-1 expression levels are measured 2-12 hours, 4-10 hours, 2-8 hours, 2-6 hours, 4-6 hours or 4-8 hours after administration.

16. The method of claim 1, wherein the HDL Therapeutic is a lipoprotein complex, optionally comprising an apolipoprotein or an apolipoprotein peptide mimic.

17. The method of claim 16, wherein
(a) the apolipoprotein is ApoA-I, ApoA-II, ApoA-IV, ApoE or a combination thereof;
(b) the peptide mimic is an ApoA-I, ApoA-II, ApoA-IV, or ApoE peptide mimic or a combination thereof; and/or
(c) the lipoprotein complex is CER-001, CSL-111, CSL-112, or ETC-216.

18. The method of claim 1, wherein the HDL Therapeutic is a small molecule, optionally a CETP inhibitor or a pantothenic acid derivative.

19. The method of claim 1 which further comprises determining a cutoff amount, optionally determined by generating a dose response curve for the HDL Therapeutic.

20. The method of claim 19, wherein the cutoff amount is 25%-75% of the dose that results in an inflection point in the dose response curve or 40%-60% of the dose that results in an inflection point in the dose response curve.

21. The method of claim 1, wherein the subject or population of subjects has an ABCA1 deficiency, and optionally wherein the subject or population of subjects is homozygous for an ABCA1 mutation or wherein the subject or population of subjects is heterozygous for an ABCA1 mutation.

22. A method of identifying a dose of a HDL Therapeutic suitable for therapy, comprising:
I. (a) administering one or more doses of a HDL Therapeutic to a subject,
(b) measuring expression levels of one or more HDL Markers in said subject's circulating monocytes, macrophages or mononuclear cells following each dose; and
(c) identifying the maximum dose that does not raise expression levels of said one or more HDL Markers by more than 0%, more than 10% or more than 20%, thereby identifying a dose of a HDL Therapeutic suitable for therapy;
II. (a) administering one or more doses of a HDL Therapeutic to a population of subjects,
(b) measuring expression levels of one or more HDL Markers in said subjects' circulating monocytes, macrophages or mononuclear cells following each dose; and
(c) identifying the maximum dose that does not raise expression levels of said one or more HDL Markers by more than 0%, more than 10% or more than 20% in said subjects, thereby identifying a dose of a HDL Therapeutic suitable for therapy;
III. identifying the highest dose of the HDL therapeutic that does not reduce cellular cholesterol efflux by more than 0%, more than 10% or more than 20%;
IV. identifying the highest dose of the HDL therapeutic that does not reduce cellular cholesterol efflux by more than 0%, more than 10% or more than 20% by
(a) administering one or more doses of a HDL Therapeutic to a subject or population of subjects;
(b) measuring cholesterol efflux in cells from said subject or population of subjects; and
(c) identifying the maximum dose that does not reduce cholesterol efflux by more than 0%, more than 10% or more than 20% in said subjects, thereby identifying a dose of a HDL Therapeutic suitable for therapy; or
V. (a) administering one or more doses of the HDL Therapeutic to a subject or to a population of subjects,
(b) measuring expression levels of one or more HDL Markers in said subject's or population's circulating monocytes, macrophages or mononuclear cells following each dose; and
(c) identifying a dose that maintains baseline expression levels or raises the expression levels of one or more HDL Markers in the subject's circulating monocytes, macrophages or mononuclear cells, thereby identifying a dose of an HDL Therapeutic suitable for therapy.

23. A method of identifying a dosing interval of a HDL Therapeutic suitable for therapy, comprising identifying the highest dose of the most frequent dosing regimen of the HDL therapeutic that does not reduce cellular cholesterol efflux by more than 0%, more than 10% or more than 20%, wherein the method optionally comprises:
(a) administering a HDL Therapeutic to a subject or population of subjects according to one or more dosing frequencies;
(b) measuring cholesterol efflux in cells from said subject or population of subjects; and
(c) identifying the maximum dosing frequency that does not reduce cholesterol efflux by more than 0%, more than 10% or more than 20% in said subjects, thereby identifying a dose of a HDL Therapeutic suitable for therapy.

24. The method of claim 23, wherein the one or more dosing frequencies includes one or more dosing frequencies selected from:
(a) administration as a 1-4 hour infusion every 2 days;
(b) administration as a 1-4 hour an infusion every 3 days;
(c) administration as a 24 hour infusion every week days; and
(d) administration as a 24 hour an infusion every two weeks.

25. The method of claim 22, wherein cholesterol efflux is measured in monocytes, macrophages or mononuclear cells from said subjects or populations of subjects.

26. A method for treating a subject with an ABCA1 deficiency, comprising administering to the subject a therapeutically effective amount of an HDL Therapeutic, which is optionally CER-001.

27. The method of claim 26, wherein the subject is heterozygous for an ABCA1 mutation or wherein the subject is homozygous for an ABCA1 mutation.

28. A method of treating a subject suffering from familial primary hypoalphalipoproteinemia, comprising:
(a) administering to the subject an HDL Therapeutic according to an induction regimen; and, subsequently
(b) administering to the subject the HDL Therapeutic according to a maintenance regimen.

29. The method of claim 28, wherein
(a) the maintenance regimen entails administering the HDL therapeutic at a lower dose, a lower frequency, or both;
(b) the subject is heterozygous for an ABCA1 mutation or wherein the subject is homozygous for an ABCA1 mutation;
(c) the subject is homozygous or heterozygous for an LCAT mutation;
(d) the subject is homozygous or heterozygous for an ApoA-I mutation;
(e) the subject is homozygous or heterozygous for an ABCG1 mutation;
(f) the subject is also treated with a lipid control medication, which is optionally atorvastatin, ezetimibe, niacin, rosuvastatin, simvastatin, aspirin, fluvastatin, lovastatin, pravastatin or a combination thereof; and/or
(g) the HDL Therapeutic is CER-001 and, optionally, the induction regimen is of a duration of 4 weeks.

30. The method of claim 29, wherein the HDL Therapeutic is CER-001 and wherein:
(a) the induction regimen comprises administering CER-001 three times a week;
(b) the dose of CER-001 administered in the induction regimen is 8-15 mg/kg (on a protein weight basis), 8 mg/kg, 12 mg/kg or 15 mg/kg;
(c) the maintenance regimen comprises administering CER-001 for at least one month, at least two months, at least three months, at least six months, at least a year, at least 18 months, at least two years, or indefinitely and/or wherein the maintenance regimen comprises administering CER-001 twice a week; and/or
(d) the dose administered in the maintenance regimen is 1-6 mg/kg (on a protein weight basis), 1 mg/kg, 3 mg/kg or 6 mg/kg.

31. The method of claim 28, wherein:
(a) the induction regimen utilizes a dose that reduces expression levels of one or more HDL Markers by 20%-80% or 40%-60%, as compared to the subject's baseline amount and/or a population average; and/or
(b) the maintenance regimen utilizes a dose that does not reduce expression levels of one or more HDL Markers by more than 20% or more than 10% as compared to the subject's baseline amount and/or a population average, optionally wherein the maintenance regimen utilizes a dose that does not reduce expression levels of one or more HDL Markers.
